SEQUENCE LISTING (84 sequences)

SEQ ID NO: 1 - DNA, 23 bp, Artificial Sequence
  sgRNA targeting site
ccaccggaagctacgacaaaagg

SEQ ID NO: 2 - DNA, 23 bp, Artificial Sequence
  sgRNA targeting site
tctctacagcgtggtttctaagg

SEQ ID NO: 3 - DNA, 23 bp, Artificial Sequence
  sgRNA targeting site
ggcttgtgggagagtggttcagg

SEQ ID NO: 4 - DNA, 23 bp, Artificial Sequence
  sgRNA targeting site
ccacgctgtagagagaggggggg

SEQ ID NO: 5 - DNA, 23 bp, Artificial Sequence
  sgRNA targeting site
aggggaggttagcgtcacttagg

SEQ ID NO: 6 - DNA, 23 bp, Artificial Sequence
  sgRNA targeting site
gaaatcgaaacttagccccaagg

SEQ ID NO: 7 - DNA, 23 bp, Artificial Sequence
  sgRNA targeting site
gcagcctgcatagcccttactgg

SEQ ID NO: 8 - DNA, 23 bp, Artificial Sequence
  sgRNA targeting site
ccctactcaccttggcaatctgg

SEQ ID NO: 9 - DNA, 24 bp, Artificial Sequence
  Upstream sequence of sgRNA-3
caccggcttgtgggagagtggttc

SEQ ID NO: 10 - DNA, 24 bp, Artificial Sequence
  Downstream sequence of sgRNA-3
aaacgaaccactctcccacaagcc

SEQ ID NO: 11 - DNA, 25 bp, Artificial Sequence
  Upstream sequence of sgRNA-6
caccggaaatcgaaacttagcccca

SEQ ID NO: 12 - DNA, 25 bp, Artificial Sequence
  Downstream sequence of sgRNA-6
aaactggggctaagtttcgatttcc

SEQ ID NO: 13 - DNA, 132 bp, Artificial Sequence
  DNA fragment containing the T7 promoter and sgRNA fragment
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 14 - DNA, 25 bp, Artificial Sequence
  PCR primer
aagatagcctagagggaaaaggtgg

SEQ ID NO: 15 - DNA, 25 bp, Artificial Sequence
  PCR primer
aggtagaaaaagggagggagaatcc

SEQ ID NO: 16 - DNA, 1560 bp, Artificial Sequence
  5′ homologous arm
cacatctgccatgaaaattggatctcaaggcaagccctggctggggaagaaggagaggcaggacagtgagcaactgatgacaggcccatcccaaggctctgggtgcttgcagtgacagcagagctgatccatggacctccccgccttaggctctctccctttgacagtgcagcaacaccaggccattggcagataggcaaaaacagtctcttttaccctgccaacacaagaggggctacccaaggatcctgctacagccctgtttaatagacagagtcacattagaagctggtggggatcccagttggggaccaagatcacctgactcactgttgcagtctgccactcttgctatcttcgtcctccctttcctgtcctcctgtctgggccactgtgtctctaatgtctatgtgtttgaatgctcatgttctacatccataagttcagcaaaccatggttcaggaaggtagttaggccactgtgcttgggtggcaaggactttaatccattgaatcatcccctagatgctgctttttaaaaatgacttattttattttaattatgtatatctattggtatgcatgtgtgtgggcacttgtgaaggctagaagcccctggatcccctgcacttggacgtacagaccacatgatataagtgctgggtatcgatcctgggtcctctggaagatcagccagtgctcttaaccgctggccatttctccagcccagatgctgctttttaactcaccaacttcagtttgtttgtttgtttgtttgtttgtattagaagtacatcaagatgggctcatgctacacatttcccagaaacaaaggccaaggaccatcaaaagccctgtccatgctaaagccacaactccgcagcatttggcagagggtgagcacctgggcctactgatgactccaagtgatgcagccatatgctactgagccacacctaggacctaggttctcagctctcctaccactagactgctgagacccgctgctctgctcaggacttgctttccagtacacaatctccctctttgaaaagtaccacacatcctggggtctcctccatctgtatctccctctgtggggaattcaagatcccattataactgacaaattcaggccgggcatggtggcacacgcctttaatcccagcactcgggaggcagaggcaggtggatttctgagttcgaggccagcctggtctacagagtgagttccaggacagccagggctatacagagaaaccctgtctcgaaaaaccaaaataaataaataaataaataaataaataactgacaagttcagctcagacatgacacttctccaaagggcttgggcactctactatgctaagctatgtggcttagcactctgtgctttagttttcttattcatcttgagttacaggctcatgtggggaataacacatgccgaacattgtgctgaactcaatagcattgtgtgtgctcaatcagtgaaatctgagcctgcaggatcccttaaggttagtcatggctgccatttttctttccaggagccacgagg

SEQ ID NO: 17 - DNA, 1399 bp, Artificial Sequence
  3′ homologous arm
ggaacagaggtctatgtactcggtaagtaagcctgtgccttcttcataccctgatgagtagcatcaggtcagagaataacatcttggatctcgtaagtgtggctcacagcaggtacaatgggaggtcaggttcacgatctaagctcaaccttcaacagatcagagaagctgaagccaggtgttagctcaaggttccagtgctagcaagaaagagggcatctgcatccctcatctgcccaccccacagccctctcttccttgcccatggttcaagtaccagtgaataccagaatgcagatcccggcaggctccttgctgcccaggcattaagcatccccacaggtgagcccaggacacaggagatgctgagaaagttctgaacctctaggaaacccagactctccaccgtcacttacacaaagactcaggataccctctccattcagaagtcagggcctgtcagtgactgggaagctctcccaaatgccaggcaggacacaaagcacccctccattatgtgaatgcctccattttactctagcatctagaaattccacaatttattgtagtcttctgtttgtagagaattcagttcttccaaactgtggacttttacaatagtgctgcactggatgctcatcccacttgtctaggacagagacgcccactgtgcaaggcctacatcacatagctgtgttcgttttaatttgtatagatgatagcagttttccctccaggcagcaaccgagtctccttgtcacctctgcacagaagcagggcaggtagcttatcctgatatgacagacaccaaagctcccaggtagtgtgtcacttgcccaggcaactacaactagcaaggggcttcctgccacatcttcatcacgtgtggcttcatgctatatcttcttcatagcattttctccttaagccagagcatactcatactctacctgtccagggtgtattaggacactgaatagccctttaggaagccatgaggaattgcccaagaccttgctatttggagcaagattggcttctgtcttcagtttctctttgtgtaaataacagtaatggctccattttgggttctatgagcgtcaagatattaacaaagccctaaatgggggggggggcgtcaaggtcattactgaggatgagaagtacagtggccaggaggacagggggctgggtttttacagtttggaaccacaacatagataagccactcctttgcctgtgtcctaggacaaatccctcgtagcagcctcggtgggctcctatgtccattccactctcgtagagtatgtggtcaggactgaagcagtgacatcttatcaaacacttagccagcacctgacttgtaggaagcatcccaccagtgggagctgtcctgaac

SEQ ID NO: 18 - DNA, 324 bp, Homo sapiens
gaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagca

SEQ ID NO: 19 - DNA, 11 bp, Mus musculus
gagccacgagg

SEQ ID NO: 20 - DNA, 16 bp, Mus musculus
ggaacagaggtctatg

SEQ ID NO: 21 - DNA, 23 bp, Artificial Sequence
  sgRNA targeting site
agttccttccccgtggctcctgg

SEQ ID NO: 22 - DNA, 23 bp, Artificial Sequence
  sgRNA targeting site
agccacggggaaggaactgaagg

SEQ ID NO: 23 - DNA, 23 bp, Artificial Sequence
  sgRNA targeting site
caccttcagttccttccccgtgg

SEQ ID NO: 24 - DNA, 23 bp, Artificial Sequence
  sgRNA targeting site
aaatcagtgtctgttgctgctgg

SEQ ID NO: 25 - DNA, 23 bp, Artificial Sequence
  sgRNA targeting site
cactttgacctccttgttgccgg

SEQ ID NO: 26 - DNA, 23 bp, Artificial Sequence
  sgRNA targeting site
ttgacctccttgttgccggtggg

SEQ ID NO: 27 - DNA, 23 bp, Artificial Sequence
  sgRNA targeting site
gggtcccaccggcaacaaggagg

SEQ ID NO: 28 - DNA, 23 bp, Artificial Sequence
  sgRNA targeting site
tgttgccggtgggacccattagg

SEQ ID NO: 29 - DNA, 23 bp, Artificial Sequence
  sgRNA targeting site
actcctctgtaccacctaatggg

SEQ ID NO: 30 - DNA, 23 bp, Artificial Sequence
  sgRNA targeting site
ctgtagatcaacagccggcttgg

SEQ ID NO: 31 - DNA, 23 bp, Artificial Sequence
  sgRNA targeting site
cgaaactgtagatcaacagccgg

SEQ ID NO: 32 - DNA, 23 bp, Artificial Sequence
  sgRNA targeting site
ctgttgatctacagtttcgcagg

SEQ ID NO: 33 - DNA, 23 bp, Artificial Sequence
  sgRNA targeting site
tctgaaacatttctaattcgagg

SEQ ID NO: 34 - DNA, 23 bp, Artificial Sequence
  sgRNA targeting site
tactactaagagaaacaatatgg

SEQ ID NO: 35 - DNA, 23 bp, Artificial Sequence
  sgRNA targeting site
ctggggtgacattactgatacgg

SEQ ID NO: 36 - DNA, 23 bp, Artificial Sequence
  sgRNA targeting site
aatgtcaccccagcagatgctgg

SEQ ID NO: 37 - DNA, 23 bp, Artificial Sequence
  sgRNA targeting site
gtagatgccagcatctgctgggg

SEQ ID NO: 38 - DNA, 23 bp, Artificial Sequence
  sgRNA targeting site
cctgacacagaaatacaatctgg

SEQ ID NO: 39 - DNA, 23 bp, Artificial Sequence
  sgRNA targeting site
cacagaaatacaatctggagggg

SEQ ID NO: 40 - DNA, 23 bp, Artificial Sequence
  sgRNA targeting site
acaatctggagggggaacagagg

SEQ ID NO: 41 - DNA, 23 bp, Artificial Sequence
  sgRNA targeting site
ggaacagaggtctatgtactcgg

SEQ ID NO: 42 - DNA, 18 bp, Artificial Sequence
  sgRNA-7 upstream sequence
gtcccaccggcaacaagg

SEQ ID NO: 43 - DNA, 22 bp, Artificial Sequence
  sgRNA-7 forward oligonucleotide
tagggtcccaccggcaacaagg

SEQ ID NO: 44 - DNA, 18 bp, Artificial Sequence
  sgRNA-7 downstream sequence
ccttgttgccggtgggac

SEQ ID NO: 45 - DNA, 22 bp, Artificial Sequence
  sgRNA-7 reverse oligonucleotide
aaacccttgttgccggtgggac

SEQ ID NO: 46 - DNA, 19 bp, Artificial Sequence
  sgRNA-17 upstream sequence
tagatgccagcatctgctg

SEQ ID NO: 47 - DNA, 23 bp, Artificial Sequence
  sgRNA-17 forward oligonucleotide
taggtagatgccagcatctgctg

SEQ ID NO: 48 - DNA, 19 bp, Artificial Sequence
  sgRNA-17 downstream sequence
cagcagatgctggcatcta

SEQ ID NO: 49 - DNA, 23 bp, Artificial Sequence
  sgRNA-17 reverse oligonucleotide
aaaccagcagatgctggcatcta

SEQ ID NO: 50 - DNA, 350 bp, Artificial Sequence
  A portion of humanized SIRP&A gene
agccacgagggaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggaacagaggtctatg

SEQ ID NO: 51 - DNA, 1206 bp, Artificial Sequence
  CDS
atggagcccgccggcccggcccctggccgcctagggccgctgctgctctgcctgctgctctccgcgtcctgtttctgtacaggagccacgagggaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtga

SEQ ID NO: 52 - DNA, 3707 bp, Artificial Sequence
  mRNA sequence of humanized SIRP&A gene
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacgagggaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccaggtacagtctttgatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 53 - protein, 513 aa, Artificial Sequence
  Protein sequence of humanized SIRP&A
MEPAGPAPGRLGPLLLCLLLSASCFCTGATREEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 54 - DNA, 25 bp, Artificial Sequence
  PCR primer
catcaagcctgttccctccttgtgt

SEQ ID NO: 55 - DNA, 25 bp, Artificial Sequence
  PCR primer
cttaaactccacgtcatcggggctc

SEQ ID NO: 56 - DNA, 26 bp, Artificial Sequence
  PCR primer
tcaaaaagaaggccacttcccccggg

SEQ ID NO: 57 - DNA, 25 bp, Artificial Sequence
  PCR primer
caagctgtagagacagatgggcagg

SEQ ID NO: 58 - DNA, 23 bp, Artificial Sequence
  Probe primer
gccctccccagccccagatttta

SEQ ID NO: 59 - DNA, 22 bp, Artificial Sequence
  Probe primer
gaagtatgcagatctctgtgat

SEQ ID NO: 60 - DNA, 21 bp, Artificial Sequence
  Probe primer
cgcccactgtgcaaggcctac

SEQ ID NO: 61 - DNA, 23 bp, Artificial Sequence
  Probe primer
gctccaaatagcaaggtcttggg

SEQ ID NO: 62 - protein, 108 aa, Artificial Sequence
  A fragment of human SIPR&A protein
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGA

SEQ ID NO: 63 - DNA, 22 bp, Artificial Sequence
  RT-PCR primer
ccccgatgacgtggagtttaag

SEQ ID NO: 64 - DNA, 25 bp, Artificial Sequence
  RT-PCR primer
tgggtagcattattaccagggaagg

SEQ ID NO: 65 - DNA, 23 bp, Artificial Sequence
  RT-PCR primer
ccccgatgacgtggagtttaagt

SEQ ID NO: 66 - DNA, 25 bp, Artificial Sequence
  RT-PCR primer
gggtagcattattaccagggaaggt

SEQ ID NO: 67 - DNA, 25 bp, Artificial Sequence
  RT-PCR primer
ttgatctacagtttcacaacagaac

SEQ ID NO: 68 - DNA, 25 bp, Artificial Sequence
  RT-PCR primer
acaacagaacactttcctcgagtta

SEQ ID NO: 69 - DNA, 22 bp, Artificial Sequence
  RT-PCR primer
tcaccatcttccaggagcgaga

SEQ ID NO: 70 - DNA, 21 bp, Artificial Sequence
  RT-PCR primer
gaaggccatgccagtgagctt

SEQ ID NO: 71 - protein, 369 aa, Mus musculus
  Mouse CD132 protein NP_038591.1
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 72 - DNA, 312 bp, Artificial Sequence
  Donor (human CD47)
aataaaacaaaatctgtagaattcacgttttgtaatgacactgtcgtcattccatgctttgttactaatatggaggcacaaaacactactgaagtatacgtaaagtggaaatttaaaggaagagatatctacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatc

SEQ ID NO: 73 - DNA, 486 bp, Artificial Sequence
  A portion of humanized CD47 gene
tatatgcagattgtaatgaaatatttttgtgtatgtattccaggttcagctcaactactgtttaataaaacaaaatctgtagaattcacgttttgtaatgacactgtcgtcattccatgctttgttactaatatggaggcacaaaacactactgaagtatacgtaaagtggaaatttaaaggaagagatatctacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatagagctgaaaaaccgcacgggtaagtgacacagtttgcctgttttgaaacgtgtgttgagatatggttgccactgtgggagtgctgtaaggtggaaccttgcagaagtc

SEQ ID NO: 74 - DNA, 1925 bp, Artificial Sequence
  mRNA of humanized CD47 gene
cccgggcagcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttaataaaacaaaatctgtagaattcacgttttgtaatgacactgtcgtcattccatgctttgttactaatatggaggcacaaaacactactgaagtatacgtaaagtggaaatttaaaggaagagatatctacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaa

SEQ ID NO: 75 - protein, 326 aa, Artificial Sequence
  Protein sequence of humanized CD47
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKNRTAFNTDQGSACSYEEEKGGCKLVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 76 - DNA, 3868 bp, Homo sapiens
  Human SIRP&A mRNA NM_080792.2
cgctcgctcgcagagaagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 77 - protein, 504 aa, Homo sapiens
  Human SIRP&A protein NP_542970.1
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 78 - DNA, 4031 bp, Mus musculus
  Mouse SIRP&A mRNA NM_007547.4
cgggaaggtgcgggcgcgaggagggggcgctcggccgggccgccctcgcgctggcctcgcgacggctccgcacagcccgcactcgctctgcgagctgtccccgctcgcgcttgctctccgatctccgtccccgctccctctccctcttcctctccccctctttccttctccctcgctatccgctcccccgcccccgtgcctctggctctgcgcctggctccctcgggtccgctcccctttcccgccggcctggcccggcgtcacgctcccggagtctccccgctcggcggcgtctcattgtgggagggggtcagatcaccccgccgggcggtggcgctggggggcagcggagggggaggggccttagtcgttcgcccgcgccgcccgcccgcctgccgagcgcgctcaccgccgctctccctccttgctctgcagccgcggcccatggagcccgccggcccggcccctggccgcctagggccgctgctgctctgcctgctgctctccgcgtcctgtttctgtacaggagccacggggaaggaactgaaggtgactcagcctgagaaatcagtgtctgttgctgctggggattcgaccgttctgaactgcactttgacctccttgttgccggtgggacccattaggtggtacagaggagtagggccaagccggctgttgatctacagtttcgcaggagaatacgttcctcgaattagaaatgtttcagatactactaagagaaacaatatggacttttccatccgtatcagtaatgtcaccccagcagatgctggcatctactactgtgtgaagttccagaaaggatcatcagagcctgacacagaaatacaatctggagggggaacagaggtctatgtactcgccaaaccttctccaccggaggtatccggcccagcagacaggggcatacctgaccagaaagtgaacttcacctgcaagtctcatggcttctctccccggaatatcaccctgaagtggttcaaagatgggcaagaactccaccccttggagaccaccgtgaaccctagtggaaagaatgtctcctacaacatctccagcacagtcagggtggtactaaactccatggatgttaattctaaggtcatctgcgaggtagcccacatcaccttggatagaagccctcttcgtgggattgctaacctgtctaacttcatccgagtttcacccaccgtgaaggtcacccaacagtccccgacgtcaatgaaccaggtgaacctcacctgccgggctgagaggttctaccccgaggatctccagctgatctggctggagaatggaaacgtatcacggaatgacacgcccaagaatctcacaaagaacacggatgggacctataattacacaagcttgttcctggtgaactcatctgctcatagagaggacgtggtgttcacgtgccaggtgaagcacgaccaacagccagcgatcacccgaaaccataccgtgctgggatttgcccactcgagtgatcaagggagcatgcaaaccttccctgataataatgctacccacaactggaatgtcttcatcggtgtgggcgtggcgtgtgctttgctcgtagtcctgctgatggctgctctctacctcctccggatcaaacagaagaaagccaaggggtcaacatcttccacacggttgcacgagcccgagaagaacgccagggaaataacccagatccaggacacaaatgacatcaacgacatcacatacgcagacctgaatctgcccaaagagaagaagcccgcaccccgggcccctgagcctaacaaccacacagaatatgcaagcattgagacaggcaaagtgcctaggccagaggataccctcacctatgctgacctggacatggtccacctcagccgggcacagccagcccccaagcctgagccatctttctcagagtatgctagtgtccaggtccagaggaagtgaatggggctgtggtctgtactaggccccatccccacaagttttcttgtcctacatggagtggccatgacgaggacatccagccagccaatcctgtccccagaaggccaggtggcacgggtcctaggaccaggggtaagggtggcctttgtcttccctccgtggctcttcaacacctcttgggcacccacgtccccttcttccggaggctgggtgttgcagaaccagagggcgaactggagaaagctgcctggaatccaagaagtgttgtgcctcggcccatcactcgtgggtctggatcctggtcttggcaaccccaggttgcgtccttgatgttccagagcttggtcttctgtgtggagaagagctcaccatctctacccaacttgagctttgggaccagactccctttagatcaaaccgccccatctgtggaagaactacaccagaagtcagcaagttttcagccaacagtgctggcctccccacctcccaggctgactagccctggggagaaggaaccctctcctcctagaccagcagagactccctgggcatgttcagtgtggccccacctcccttccagtcccagcttgcttcctccagctagcactaactcagcagcatcgctctgtggacgcctgtaaattattgagaaatgtgaactgtgcagtcttaaagctaaggtgttagaaaatttgatttatgctgtttagttgttgttgggtttcttttctttttaatttctttttcttttttgattttttttctttcccttaaaacaacagcagcagcatcttggctctttgtcatgtgttgaatggttgggtcttgtgaagtctgaggtctaacagtttattgtcctggaaggattttcttacagcagaaacagatttttttcaaattcccagaatcctgaggaccaagaaggatccctcagctgctacttccagcacccagcgtcactgggacgaaccaggccctgttcttacaaggccacatggctggccctttgcctccatggctactgtggtaagtgcagccttgtctgacccaatgctgacctaatgttggccattccacattgaggggacaaggtcagtgatgccccccttcactcacaagcacttcagaggcatgcagagagaagggacactcggccagctctctgaggtaatcagtgcaaggaggagtccgttttttgccagcaaacctcagcaggatcacactggaacagaacctggtcatacctgtgacaacacagctgtgagccagggcaaaccacccactgtcactggctcgagagtctgggcagaggctctgaccctccaccctttaaactggatgccggggcctggctgggcccaatgccaagtggttatggcaaccctgactatctggtcttaacatgtagctcaggaagtggaggcgctaatgtccccaatccctggggattcctgattccagctattcatgtaagcagagccaacctgcctatttctgtaggtgcgactgggatgttaggagcacagcaaggacccagctctgtagggctggtgacctgatacttctcataatggcatctagaagttaggctgagttggcctcactggcccagcaaaccagaacttgtctttgtccgggccatgttcttgggctgtcttctaattccaaagggttggttggtaaagctccacccccttctcctctgcctaaagacatcacatgtgtatacacacacgggtgtatagatgagttaaaagaatgtcctcgctggcatcctaattttgtcttaagtttttttggagggagaaaggaacaaggcaagggaagatgtgtagctttggctttaaccaggcagcctgggggctcccaagcctatggaaccctggtacaaagaagagaacagaagcgccctgtgaggagtgggatttgtttttctgtagaccagatgagaaggaaacaggccctgttttgtacatagttgcaacttaaaatttttggcttgcaaaatatttttgtaataaagatttctgggtaacaataaaaaaaaaaaaaaa

SEQ ID NO: 79 - protein, 509 aa, Mus musculus
  Mouse SIRP&A protein NP_031573.2
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 80 - DNA, 5346 bp, Homo sapiens
  Human CD47 mRNA NM_001777.3
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 81 - protein, 323 aa, Homo sapiens
  Human CD47 protein NP_001768.1
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 82 - DNA, 1928 bp, Mus musculus
  Mouse CD47 mRNA NM_010581.3
cccgggcagcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaaaaaaaaaa

SEQ ID NO: 83 - protein, 324 aa, Mus musculus
  Mouse CD47 protein NP_034711.1
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 84 - DNA, 1663 bp, Mus musculus
  Mouse CD132 cDNA NM_013563.4
aggaaatgtatgggtggggagggcttgtgggagagtggttcagggttctgacacagactacacccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatca

(19) United States
(12) Patent Application Publication
(10) Pub. No.: US 2023/0058049 A1
(43) Pub. Date: Feb. 23, 2023

(54) GENETICALLY MODIFIED IMMUNODEFICIENT NON-HUMAN ANIMAL WITH HUMAN OR CHIMERIC SIRPa/CD47

(71) Applicant: Biocytogen Pharmaceuticals (Beijing) Co., Ltd., Daxing District, Beijing (CN)

(72) Inventors: Yuelei Shen, Beijing (CN); Yang Bai, Beijing (CN); Jiawei Yao, Beijing (CN); Chaoshe Guo, Beijing (CN); Meiling Zhang, Beijing (CN); Rui Huang, Beijing (CN); Yanan Guo, Beijing (CN); Chengzhang Shang, Beijing (CN)

(21) Appl. No.: 17/790,059

(22) PCT Filed: Dec. 31, 2020
(86) PCT No.: PCT/CN2020/142546

§ 371 Date: Jun. 29, 2022

(30) Foreign Application Priority Data
Dec. 31, 2019 (CN) 201911408385.4

(51) Int. Cl. A01K 67/027; C12N 15/85

(52) U.S. Cl. CPC A01K 67/0276; A01K 67/0271; A01K 67/0278; C12N 15/8509; A01K 2207/12; A01K 2207/15; A01K 2217/072; A01K 2217/075; A01K 2227/105; A01K 2267/0387

(57) ABSTRACT

This disclosure relates to genetically modified immunodeficient animals which express a human or chimeric (e.g., humanized) SIRPα and/or human or chimeric (e.g., humanized) CD47, and methods of use thereof.

### TECHNICAL FIELD

This disclosure relates to genetically modified immunodeficient animals which express a human or chimeric (e.g., humanized) SIRPα and/or human or chimeric (e.g., humanized) CD47, and methods of use thereof.

### BACKGROUND

The immune system has developed multiple mechanisms to prevent deleterious activation of T cells. One such mechanism is the intricate balance between positive and negative costimulatory signals delivered to T cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, thus the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an immune-deficient animal model with (1) human SIRPα or chimeric SIRPα; and/or (2) human CD47 or chimeric CD47. In some embodiments, the animal model have a CD132 gene knockout and can express human SIRPα or chimeric SIRPα (e.g., humanized SIRPα) protein in its body. In some embodiments, the animal model have a CD132 gene knockout and can express human CD47 or chimeric CD47 (e.g., humanized CD47) protein in its body. The animal can be used in the studies on the function of SIRPα gene and/or CD47 gene, and can be used in the screening and evaluation of anti-human SIRPα and anti-CD47 antibodies. In addition, the animal models prepared by the methods described herein can be used in drug screening, pharmacodynamics studies, treatments for immune-related diseases (e.g., autoimmune disease), and cancer therapy for human SIRPα or CD47 target sites; they can also be used to facilitate the development and design of new drugs, and save time and cost. In summary, this disclosure provides a powerful tool for studying the function of SIRPα protein and CD47 protein, and a platform for screening cancer drugs. In one aspect, the disclosure is related to a genetically-modified, non-human animal whose genome comprises at least one chromosome comprising a sequence encoding a human or chimeric SIRPα. In some embodiments, the animal is immune-deficient.

In some embodiments, the genome of the animal comprises a disruption in the animal's endogenous CD132 gene. In some embodiments, the sequence encoding the human or chimeric SIRPα is operably linked to an endogenous regulatory element at the endogenous SIRPα gene locus in the at least one chromosome. In some embodiments, the sequence encoding a human or chimeric SIRPα comprises a sequence encoding an amino acid sequence that is at least 70%, 75%, 80%, 85%, 90%, 95%, 99%, or 100% identical to human SIRPα (NP 542970.1; SEQ ID NO: 77). In some embodiments, the sequence encoding a human or chimeric SIRPα comprises a sequence encoding an amino acid sequence that is at least 70%, 75%, 80%, 85%, 90%, 95%, 99%, or 100% identical to SEQ ID NO: 53. In some embodiments, the sequence encoding a human or chimeric SIRPα comprises a sequence that is at least 70%, 75%, 80%, 85%, 90%, 95%, 99%, or 100% identical to amino acids 31-138 of SEQ ID NO: 77.

In some embodiments, the animal is a mammal, e.g., a monkey, a rodent or a mouse. In some embodiments, the animal is a mouse.

In some embodiments, the animal does not express endogenous SIRPα.

In some embodiments, the animal has one or more cells expressing human or chimeric SIRPα. In some embodiments, the genome of the animal comprises a replacement of a sequence encoding a region of endogenous SIRPα with a sequence encoding a corresponding region of human SIRPα at an endogenous SIRPα gene locus. In some embodiments, the replaced locus is the extracellular domain of SIRPα. In some embodiments, the replaced locus is the extracellular N-terminal IgV domain of SIRPα. In some embodiments, the animal is a mouse, and the replaced endogenous SIRPα region is exon 2 of the endogenous mouse SIRPα gene. In some embodiments, the sequence encoding a corresponding region of human SIRPα comprises at least 100, 200, or 300 nucleotides of exon 3 of a human SIRPα gene. In some embodiments, the animal is homozygous with respect to the replacement at the endogenous SIRPα gene locus. In some embodiments, the animal is heterozygous with respect to the replacement at the endogenous SIRPα gene locus.

In some embodiments, the disruption of the endogenous CD132 gene comprises deletion of exon 2 of the endogenous CD132 gene. In some embodiments, the disruption of the endogenous CD132 gene comprises deletion of exon 1, or part thereof of the endogenous CD132 gene. In some embodiments, the disruption of the endogenous CD132 gene further comprises deletion of one or more exons or part of exons selected from the group consisting of exon 3, exon 4, exon 5, exon 6, exon 7, and exon 8 of the endogenous CD132 gene.

In some embodiments, the disruption of the endogenous CD132 gene comprises deletion of exons 1-8 of the endogenous CD132 gene. In some embodiments, the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene. In some embodiments, the disruption consists of deletion of more than 150 nucleotides in exon 1, deletion of the entirety of intron 1, exon 2, intron 2, exon 3, intron 3, exon 4, intron 4, exon 5, intron 5, exon 6, intron 6, exon 7, intron 7, and deletion of more than 250 nucleotides in exon 8. In some embodiments, the animal is homozygous with respect to the disruption of the endogenous CD132 gene. In some embodiments, the animal is heterozygous with respect to the disruption of the endogenous CD132 gene.

In some embodiments, the disruption prevents the expression of functional CD132 protein. In some embodiments, the length of the remaining exon sequences at the endogenous CD132 gene locus is less than 30% of the total length of all exon sequences of the endogenous CD132 gene. In some embodiments, the length of the remaining sequences at that the endogenous CD132 gene locus is less than 15% of the full sequence of the endogenous CD132 gene.

In some embodiments, the animal further comprises a sequence encoding an additional human or chimeric protein (e.g., CD47, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, tumor necrosis factor receptor superfamily member 9 (4-1BB), or TNF Receptor Superfamily Member 4 (OX40)). In some embodiments, the additional human or chimeric protein is CD47 and/or PD-1.

In one aspect, the disclosure is related to a genetically-modified, non-human animal whose genome comprises at least one chromosome comprising a sequence encoding a human or chimeric CD47. In some embodiments, the animal is immune-deficient.

In some embodiments, the genome of the animal comprises a disruption in the animal's endogenous CD132 gene.

In some embodiments, the sequence encoding the human or chimeric CD47 is operably linked to an endogenous regulatory element at the endogenous CD47 gene locus in the at least one chromosome.

In some embodiments, the sequence encoding a human or chimeric CD47 comprises a sequence encoding an amino acid sequence that is at least 70%, 75%, 80%, 85%, 90%, 95%, 99%, or 100% identical to human CD47 (NP_001768.1; SEQ ID NO: 81).

In some embodiments, the sequence encoding a human or chimeric CD47 comprises a sequence encoding an amino acid sequence that is at least 70%, 75%, 80%, 85%, 90%, 95%, 99%, or 100% identical to SEQ ID NO: 75.

In some embodiments, the sequence encoding a human or chimeric CD47 comprises a sequence that is at least 70%, 75%, 80%, 85%, 90%, 95%, 99%, or 100% identical to amino acids 23-126 of SEQ ID NO: 81.

In some embodiments, the animal is a mammal, e.g., a monkey, a rodent or a mouse. In some embodiments, the animal is a mouse.

In some embodiments, the animal does not express endogenous CD47.

In some embodiments, the animal has one or more cells expressing human or chimeric CD47.

In some embodiments, the genome of the animal comprises a replacement of a sequence encoding a region of endogenous CD47 with a sequence encoding a corresponding region of human CD47 at an endogenous CD47 gene locus.

In some embodiments, the replaced locus is the extracellular N-terminal IgV domain of CD47.

In some embodiments, the animal is a mouse, and the replaced endogenous CD47 region is exon 2 of the endogenous mouse CD47 gene.

In some embodiments, the sequence encoding the corresponding region of CD47 comprises at least 100, 200, or 300 nucleotides of exon 2 of a human CD47 gene.

In some embodiments, the animal is homozygous with respect to the replacement at the endogenous SIRPα gene locus. In some embodiments, the animal is heterozygous with respect to the replacement at the endogenous SIRPα gene locus.

In some embodiments, the disruption of the endogenous CD132 gene comprises deletion of exon 2 of the endogenous CD132 gene.

In some embodiments, the disruption of the endogenous CD132 gene comprises deletion of exon 1 or part thereof of the endogenous CD132 gene.

In some embodiments, the disruption of the endogenous CD132 gene further comprises deletion of one or more exons or part of exons selected from the group consisting of exon 3, exon 4, exon 5, exon 6, exon 7, and exon 8 of the endogenous CD132 gene.

In some embodiments, the disruption of the endogenous CD132 gene comprises deletion of exons 1-8 of the endogenous CD132 gene.

In some embodiments, the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene.

In some embodiments, the disruption consists of deletion of more than 150 nucleotides in exon 1, deletion of the entirety of intron 1, exon 2, intron 2, exon 3, intron 3, exon 4, intron 4, exon 5, intron 5, exon 6, intron 6, exon 7, intron 7, and deletion of more than 250 nucleotides in exon 8.

In some embodiments, the animal is homozygous with respect to the disruption of the endogenous CD132 gene. In some embodiments, the animal is heterozygous with respect to the disruption of the endogenous CD132 gene.

In some embodiments, the disruption prevents the expression of functional CD132 protein.

In some embodiments, the length of the remaining exon sequences at the endogenous CD132 gene locus is less than 30% of the total length of all exon sequences of the endogenous CD132 gene. In some embodiments, the length of the remaining sequences at that the endogenous CD132 gene locus is less than 15% of the full sequence of the endogenous CD132 gene.

In some embodiments, the animal further comprises a sequence encoding an additional human or chimeric protein (e.g., SIRPα, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, tumor necrosis factor receptor superfamily member 9 (4-1BB), or TNF Receptor Superfamily Member 4 (OX40)). In some embodiments, the additional human or chimeric protein is SIRPα and/or PD-1.

In some embodiments, the animal does not have functional T cells or B cells.

In one aspect, the disclosure is related to a method of determining effectiveness of an agent targeting the CD47/SIRPα axis for the treatment of cancer, comprising: administering the agent to the animal as described herein; and determining the inhibitory effects of the agent to the tumor. In some embodiments, the animal has a tumor.

In some embodiments, the agent comprises or consists of an anti-SIRPα antibody and/or an anti-CD47 antibody.

In some embodiments, the animal comprises one or more tumor cells that express CD47.

In some embodiments, the tumor comprises one or more cancer cells that are injected into the animal.

In some embodiments, determining the inhibitory effects of the anti-SIRPα antibody to the tumor involves measuring the tumor volume in the animal.

In some embodiments, the tumor cells are melanoma cells, non-small cell lung carcinoma (NSCLC) cells, small cell lung cancer (SCLC) cells, non-Hodgkin lymphoma cells, bladder cancer cells, prostate cancer cells, breast cancer cells, ovarian cancer cells, colorectal cancer cells, and/or refractory solid tumor cells.

In one aspect, the disclosure is related to a method of determining effectiveness of an agent and an additional therapeutic agent for the treatment of a tumor, comprising administering the agent and the additional therapeutic agent to the animal as described herein, and determining the inhibitory effects on the tumor. In some embodiments, the animal has a tumor.

In some embodiments, the agent is an anti-SIRPα antibody and/or the anti-CD47 antibody.

In some embodiments, the additional therapeutic agent is an anti-PD-1 antibody, an anti-PD-L1 antibody, an anti-CTLA4 antibody, an anti-CD20 antibody, an anti-EGFR antibody, or an anti-CD319 antibody.

In one aspect, the disclosure is related to a method of determining toxicity of an agent, the method comprising administering the anti-SIRPα antibody or the anti-CD47 antibody to the animal as described herein; and determining weight change of the animal.

In some embodiments, the agent is an anti-SIRPα antibody or an anti-CD47 antibody.

In some embodiments, the method described herein further comprises performing a blood test (e.g., determining red blood cell count). In one aspect, the disclosure is related to a method of evaluating the effect of an agent targeting the CD47/SIRPα axis on phagocytosis, comprising: administering the agent to the animal as described herein. In some embodiments, the animal has a tumor.

In some embodiments, phagocytosis is induced by granulocytes (e.g., neutrophils, basophils, eosinophils, or mast cells) or macrophages.

In some embodiments, the agent is an anti-SIRPα antibody and/or an anti-CD47 antibody.

In one aspect, the disclosure is related to a method of producing an animal comprising a human hemato-lymphoid system, the method comprising: engrafting a population of cells comprising human hematopoietic cells or human peripheral blood cells into the animal as described herin.

In some embodiments, the human hemato-lymphoid system comprises human cells selected from the group consisting of hematopoietic stem cells, myeloid precursor cells, myeloid cells, dendritic cells, monocytes, granulocytes, neutrophils, mast cells, lymphocytes, and platelets.

In some embodiments, the method described herein further comprises: irradiating the animal prior to the engrafting.

In one aspect, the disclosure is related to a protein comprising an amino acid sequence. In some embodiments, the amino acid sequence is one of the following:
(a) an amino acid sequence set forth in SEQ ID NO: 53, 62, or 75;
(b) an amino acid sequence that is at least 90%, 91%, 92%, 93%, 94%, 95%, 96%, 97%, 98%, or 99% identical to SEQ ID NO: 53, 62, or 75;
(c) an amino acid sequence that is different from the amino acid sequence set forth in SEQ ID NO: 53, 62, or 75 by no more than 10, 9, 8, 7, 6, 5, 4, 3, 2 or 1 amino acid; and
(d) an amino acid sequence that comprises a substitution, a deletion and/or insertion of one, two, three, four, five or more amino acids to the amino acid sequence set forth in SEQ ID NO: 53, 62, or 75.

In one aspect, the disclosure is related to a nucleic acid comprising a nucleotide sequence. In some embodiments, the nucleotide sequence is one of the following:
(a) a sequence that encodes the protein as described herein;
(b) SEQ ID NO: 18, 50, 51, or 52;
(c) SEQ ID NO: 72, 73 or 74; and
(d) a sequence that is at least 90%, 91%, 92%, 93%, 94%, 95%, 96%, 97%, 98%, or 99% identical to SEQ ID NO: 18, 50, 51, 52, 72, 73, or 74.

In one aspect, the disclosure is related to a cell comprising the protein and/or the nucleic acid as described herein. In one aspect, the disclosure is related to an animal comprising the protein of and/or the nucleic acid as described herein.

In some embodiments, the animal has one or more cells expressing human or chimeric SIRPα, and the expressed human or chimeric SIRPα can bind to CD47 (e.g., human or endogenous CD47). In some embodiments, the animal has one or more cells expressing human or chimeric SIRPα, and the expressed human or chimeric SIRPα cannot bind to CD47 (e.g., human or endogenous CD47).

In another aspect, the disclosure is related to a genetically-modified, non-human animal, wherein the genome of the animal comprises a replacement of a sequence encoding a region of endogenous SIRPα with a sequence encoding a corresponding region of human SIRPα at an endogenous SIRPα gene locus.

In some embodiments, the sequence encoding the corresponding region of human SIRPα is operably linked to an endogenous regulatory element at the endogenous SIRPα locus, and one or more cells of the animal expresses a chimeric SIRPα.

In some embodiments, the animal does not express endogenous SIRPα. In some embodiments, the replaced locus is the extracellular region of SIRPα.

In some embodiments, the animal has one or more cells expressing a chimeric SIRPα having an extracellular region, wherein the extracellular region comprises a sequence that is at least 50%, 60%, 70%, 80%, 90%, 95%, or 99% identical to the extracellular region of human SIRPα.

In some embodiments, the extracellular region of the chimeric SIRPα has a sequence that has at least 10, 20, 30, 40, 50, 60, 70, 80, 90, 100, 200, 300, 400, 500, 600, 700, 800, 900, or 1000 contiguous amino acids that are identical to a contiguous sequence present in the extracellular region of human SIRPα.

In some embodiments, the animal is a mouse, and the replaced endogenous SIRPα locus is exon 1, exon 2, exon 3, exon 4, exon 5, exon 6, exon 7, and/or exon 8 of the endogenous mouse SIRPα gene.

In another aspect, the disclosure is related to methods for making a genetically-modified, non-human animal. The methods involve replacing in at least one cell of the animal, at an endogenous SIRPα gene locus, a sequence encoding a region of an endogenous SIRPα with a sequence encoding a corresponding region of human SIRPα.

In some embodiments, the sequence encoding the corresponding region of human SIRPα comprises exon 3 of a human SIRPα gene.

In some embodiments, the sequence encoding the corresponding region of SIRPα comprises at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 nucleotides of exon 3 of a human SIRPα gene.

In some embodiments, the sequence encoding the corresponding region of human SIRPα encodes a sequence that is at least 90% identical to amino acids 31-138 of SEQ ID NO: 77.

In some embodiments, the locus is located within the extracellular region of SIRPα.

In some embodiments, the animal is a mouse, and the locus is exon 1, exon 2, exon 3, exon 4, exon 5, exon 6, exon 7, and/or exon 8 of the mouse SIRPα gene (e.g., exon 2).

In another aspect, the disclosure is also related to a non-human animal comprising at least one cell comprising a nucleotide sequence encoding a chimeric SIRPα polypeptide, wherein the chimeric SIRPα polypeptide comprises at least 10, 20, 30, 40, 50, 60, 70, 80, 90, or 100 contiguous amino acid residues that are identical to the corresponding contiguous amino acid sequence of a human SIRPα, wherein the animal expresses the chimeric SIRPα.

In some embodiments, the chimeric SIRPα polypeptide has at least 10, 20, 30, 40, 50, 60, 70, 80, 90, or 100 contiguous amino acid residues that are identical to the corresponding contiguous amino acid sequence of a human SIRPα extracellular region.

In some embodiments, the chimeric SIRPα polypeptide comprises a sequence that is at least 90%, 95%, or 99% identical to amino acids 31-138 of SEQ ID NO: 77.

In some embodiments, the nucleotide sequence is operably linked to an endogenous SIRPα regulatory element of the animal.

In some embodiments, the chimeric SIRPα polypeptide comprises an endogenous SIRPα transmembrane region and/or a cytoplasmic region.

In some embodiments, the nucleotide sequence is integrated to an endogenous SIRPα gene locus of the animal.

In some embodiments, the chimeric SIRPα has at least one mouse SIRPα activity and/or at least one human SIRPα activity.

In another aspect, the disclosure is also related to methods of making a genetically-modified mouse cell that expresses a chimeric SIRPα. The methods involve replacing, at an endogenous mouse SIRPα gene locus, a nucleotide sequence encoding a region of mouse SIRPα with a nucleotide sequence encoding a corresponding region of human SIRPα, thereby generating a genetically-modified mouse cell that includes a nucleotide sequence that encodes the chimeric SIRPα, wherein the mouse cell expresses the chimeric SIRPα.

In some embodiments, the chimeric SIRPα comprises: an extracellular region of human SIRPα; a transmembrane region of mouse SIRPα; and/or a cytoplasmic region of mouse SIRPα.

In some embodiments, the nucleotide sequence encoding the chimeric SIRPα is operably linked to an endogenous SIRPα regulatory region, e.g., promoter.

In some embodiments, provided herein are cells comprising the nucleic acids disclosed herein. In some embodiments, provided herein are animals having the nucleic acids disclosed herein.

In another aspect, the disclosure also provides a genetically-modified, non-human animal whose genome comprise a disruption in the animal's endogenous SIRPα gene, wherein the disruption of the endogenous SIRPα gene comprises deletion of exon 2 or part thereof of the endogenous SIRPα gene.

In some embodiments, the disruption of the endogenous SIRPα gene further comprises deletion of one or more exons or part of exons selected from the group consisting of exon 1, exon 3, exon 4, exon 5, exon 6, exon 7, and/or exon 8 of the endogenous SIRPα gene.

In some embodiments, the disruption of the endogenous SIRPα gene further comprises deletion of one or more introns or part of introns selected from the group consisting of intron 1, intron 2, intron 3, intron 4, intron 5, intron 6, and intron 7 of the endogenous SIRPα gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous SIRPα gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, and/or exon 8 (e.g., deletion of at least 300 nucleotides of exon 2).

In some embodiments, the mice described in the present disclosure can be mated with the mice containing other human or chimeric genes (e.g., chimeric CD47, chimeric PD-1, chimeric PD-L1, chimeric CTLA-4, or other immunomodulatory factors), so as to obtain a mouse expressing two or more human or chimeric proteins. The mice can also, e.g., be used for screening antibodies in the case of a combined use of drugs, as well as evaluating the efficacy of the combination therapy.

In one aspect, the disclosure relates to a targeting vector, including a) a DNA fragment homologous to the 5′ end of a region to be altered (5′ homologous arm), which is selected from the SIRPα gene genomic DNAs in the length of 100 to 10,000 nucleotides; b) a desired/donor DNA sequence encoding a donor region; and c) a second DNA fragment homologous to the 3′ end of the region to be altered (3′ homologous arm), which is selected from the SIRPα gene genomic DNAs in the length of 100 to 10,000 nucleotides.

In some embodiments, a) the DNA fragment homologous to the 5′ end of a region to be altered (5′ homologous arm) is selected from the nucleotide sequences that have at least 90% homology to the NCBI accession number NC_000068.7; c) the DNA fragment homologous to the 3′ end of the region to be altered (3′ homologous arm) is selected from the nucleotide sequences that have at least 90% homology to the NCBI accession number NC_000068.7.

In some embodiments, a) the DNA fragment homologous to the 5′ end of a region to be altered (5′ homologous arm) is selected from the nucleotides from the position 129607346 to the position 129608914 of the NCBI accession number NC_000068.7; c) the DNA fragment homologous to the 3′ end of the region to be altered (3′ homologous arm) is selected from the nucleotides from the position 129609239 to the position 129610638 of the NCBI accession number NC_000068.7.

In some embodiments, a length of the selected genomic nucleotide sequence is about 3 kb, 3.5 kb, 4 kb, 4.5 kb, or 5 kb. In some embodiments, the region to be altered is exon 1, exon 2, exon 3, exon 4, exon 5, exon 6, exon 7, and/or exon 8 of mouse SIRPα gene.

In some embodiments, the sequence of the 5′ homologous arm is at least 90%, 95%, or 100% identical to SEQ ID NO: 16. In some embodiments, the sequence of the 3′ homologous arm is at least 90%, 95%, or 100% identical to SEQ ID NO: 17.

In some embodiments, the targeting vector further includes a selectable gene marker. In some embodiments, the target region is derived from human. In some embodiments, the target region is a part or entirety of the nucleotide sequence of a humanized SIRPα. In some embodiments, the nucleotide sequence is shown as one or more of exon 1, exon 2, exon 3, exon 4, exon 5, exon 6, exon 7, exon 8, and exon 9 of the human SIRPα.

In some embodiments, the nucleotide sequence of the human SIRPα encodes the human SIRPα protein with the NCBI accession number NP 542970.1 (SEQ ID NO: 77). In some embodiments, the nucleotide sequence of the human SIRPα is selected from the nucleotides from the position 1915110 to the position 1915433 of NC_000020.11.

The disclosure also relates to a cell including the targeting vector as described herein.

The disclosure also relates to a method for establishing a genetically-modified non-human animal expressing two human or chimeric (e.g., humanized) genes. The method includes the steps of

(a) using the method for establishing a SIRPα gene humanized animal model to obtain a SIRPα gene genetically modified humanized mouse;

(b) mating the SIRPα gene genetically modified humanized mouse obtained in step (a) with another humanized mouse, and then screening to obtain a double humanized mouse model.

In some embodiments, in step (b), the SIRPα gene genetically modified humanized mouse obtained in step (a) is mated with a CD47 humanized mouse to obtain a SIRPα and CD47 double humanized mouse model.

The disclosure also relates to non-human mammal generated through the methods as described herein.

In some embodiments, the genome thereof contains human gene(s).

In some embodiments, the non-human mammal is a rodent. In some embodiments, the non-human mammal is a mouse.

In some embodiments, the non-human mammal expresses a protein encoded by a humanized SIRPα gene.

The disclosure also relates to an offspring of the non-human mammal.

In another aspect, the disclosure relates to a tumor bearing non-human mammal model, characterized in that the non-human mammal model is obtained through the methods as described herein.

In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to a SIRPα genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

In some embodiments, the animal has one or more cells expressing human or chimeric CD47, and the expressed human or chimeric CD47 can bind to endogenous SIRPα. In some embodiments, the animal has one or more cells expressing human or chimeric CD47, and the expressed human or chimeric CD47 cannot bind to endogenous SIRPα.

In another aspect, the disclosure is related to a genetically-modified, non-human animal, wherein the genome of the animal comprises a replacement of a sequence encoding a region of endogenous CD47 with a sequence encoding a corresponding region of human CD47 at an endogenous CD47 gene locus.

In some embodiments, the sequence encoding the corresponding region of human CD47 is operably linked to an endogenous regulatory element at the endogenous CD47 locus, and one or more cells of the animal expresses a chimeric CD47.

In some embodiments, the animal does not express endogenous CD47. In some embodiments, the replaced locus is the extracellular N-terminal IgV domain of CD47.

In some embodiments, the animal has one or more cells expressing a chimeric CD47 having an extracellular N-terminal IgV domain, wherein the extracellular N-terminal IgV domain comprises a sequence that is at least 50%, 60%, 70%, 80%, 90%, 95%, or 99% identical to the extracellular N-terminal IgV domain of human CD47.

In some embodiments, the extracellular N-terminal IgV domain of the chimeric CD47 has a sequence that has at least 10, 20, 30, 40, 50, 60, 70, 80, 90, or 100 contiguous amino acids that are identical to a contiguous sequence present in the extracellular N-terminal IgV domain of human CD47.

In some embodiments, the animal is a mouse, and the replaced endogenous CD47 locus is exon 1, exon 2, exon 3, exon 4, exon 5, exon 6, exon 7, exon 8, exon 9, and/or exon 10 of the endogenous mouse CD47 gene.

In some embodiments, the animal is heterozygous with respect to the replacement at the endogenous CD47 gene locus. In some embodiments, the animal is homozygous with respect to the replacement at the endogenous CD47 gene locus.

In another aspect, the disclosure is related to methods for making a genetically-modified, non-human animal. The methods involve replacing in at least one cell of the animal, at an endogenous CD47 gene locus, a sequence encoding a region of an endogenous CD47 with a sequence encoding a corresponding region of human CD47.

In some embodiments, the sequence encoding the corresponding region of human CD47 comprises exon 2 of a human CD47 gene.

In some embodiments, the sequence encoding the corresponding region of CD47 comprises at least 100, 150, 200, 250, or 300 nucleotides of exon 2 of a human CD47 gene.

In some embodiments, the sequence encoding the corresponding region of human CD47 encodes a sequence that is at least 90% identical to amino acids 23-126 of SEQ ID NO: 63.

In some embodiments, the locus is located within the extracellular N-terminal IgV domain of CD47.

In some embodiments, the animal is a mouse, and the locus is exon 1, exon 2, exon 3, exon 4, exon 5, exon 6, exon 7, exon 8, exon 9, and/or exon 10 of the mouse CD47 gene (e.g., exon 2).

In another aspect, the disclosure is also related to a non-human animal comprising at least one cell comprising a nucleotide sequence encoding a chimeric CD47 polypeptide, wherein the chimeric CD47 polypeptide comprises at least 50 contiguous amino acid residues that are identical to the corresponding contiguous amino acid sequence of a human CD47, wherein the animal expresses the chimeric CD47.

In some embodiments, the chimeric CD47 polypeptide has at least 50 contiguous amino acid residues that are identical to the corresponding contiguous amino acid sequence of a human CD47 extracellular N-terminal IgV domain.

In some embodiments, the chimeric CD47 polypeptide comprises a sequence that is at least 90%, 95%, or 99% identical to amino acids 23-126 of SEQ ID NO: 63.

In some embodiments, the nucleotide sequence is operably linked to an endogenous CD47 regulatory element of the animal.

In some embodiments, the chimeric CD47 polypeptide comprises five endogenous CD47 transmembrane regions and/or an endogenous CD47 C-terminal intracellular tail.

In some embodiments, the nucleotide sequence is integrated to an endogenous CD47 gene locus of the animal.

In some embodiments, the chimeric CD47 has at least one mouse CD47 activity and/or at least one human CD47 activity.

In another aspect, the disclosure is also related to methods of making a genetically-modified mouse cell that expresses a chimeric CD47. The methods involve replacing, at an endogenous mouse CD47 gene locus, a nucleotide sequence encoding a region of mouse CD47 with a nucleotide sequence encoding a corresponding region of human CD47, thereby generating a genetically-modified mouse cell that includes a nucleotide sequence that encodes the chimeric CD47, wherein the mouse cell expresses the chimeric CD47.

In some embodiments, the chimeric CD47 comprises: an extracellular N-terminal IgV domain of human CD47; and one or more transmembrane domains of mouse CD47 and/or a C-terminal intracellular tail of mouse CD47.

In some embodiments, the nucleotide sequence encoding the chimeric CD47 is operably linked to an endogenous CD47 regulatory region, e.g., promoter.

In some embodiments, provided herein are cells comprising the nucleic acids disclosed herein. In some embodiments, provided herein are animals having the nucleic acids disclosed herein.

In another aspect, the disclosure also provides a genetically-modified, non-human animal whose genome comprise a disruption in the animal's endogenous CD47 gene, wherein the disruption of the endogenous CD47 gene comprises deletion of exon 2 or part thereof of the endogenous CD47 gene.

In some embodiments, the disruption of the endogenous CD47 gene further comprises deletion of one or more exons or part of exons selected from the group consisting of exon 1, exon 3, exon 4, exon 5, exon 6, exon 7, exon 8, exon 9, and exon 10 of the endogenous CD47 gene.

In some embodiments, the disruption of the endogenous CD47 gene further comprises deletion of one or more introns or part of introns selected from the group consisting of intron 1, intron 2, intron 3, intron 4, intron 5, intron 6, intron 7, intron 8, and intron 9 of the endogenous CD47 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous CD47 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, exon 8, exon 9, or exon 10 (e.g., deletion of at least 300 nucleotides of exon 2).

In some embodiments, the mice described in the present disclosure can be mated with the mice containing other human or chimeric genes (e.g., chimeric SIRPα, chimeric PD-1, chimeric PD-L1, chimeric CTLA-4, or other immunomodulatory factors), so as to obtain a mouse expressing two or more human or chimeric proteins. The mice can also, e.g., be used for screening antibodies in the case of a combined use of drugs, as well as evaluating the efficacy of the combination therapy.

In one aspect, the disclosure relates to a targeting vector, including a) a DNA fragment homologous to the 5′ end of a region to be altered (5′ homologous arm), which is selected from the CD47 gene genomic DNAs in the length of 100 to 10,000 nucleotides; b) a desired/donor DNA sequence encoding a donor region; and c) a second DNA fragment homologous to the 3′ end of the region to be altered (3′ homologous arm), which is selected from the CD47 gene genomic DNAs in the length of 100 to 10,000 nucleotides.

In some embodiments, a) the DNA fragment homologous to the 5′ end of a region to be altered (5′ homologous arm) is selected from the nucleotide sequences that have at least 90% homology to the NCBI accession number NC_000082.6; c) the DNA fragment homologous to the 3′ end of the region to be altered (3′ homologous arm) is selected from the nucleotide sequences that have at least 90% homology to the NCBI accession number NC_000082.6.

In some embodiments, a) the DNA fragment homologous to the 5′ end of a region to be altered (5′ homologous arm) is selected from the nucleotides from the position 49866727 to the position 49867784 of the NCBI accession number NC_000082.6; c) the DNA fragment homologous to the 3′ end of the region to be altered (3′ homologous arm) is selected from the nucleotides from the position 49868091 to the position 49869239 of the NCBI accession number NC_000082.6.

In some embodiments, a length of the selected genomic nucleotide sequence is about 3 kb, 3.5 kb, 4 kb, 4.5 kb, or 5 kb. In some embodiments, the region to be altered is exon 1, exon 2, exon 3, exon 4, exon 5, exon 6, exon 7, exon 8, exon 9, and/or exon 10 of mouse CD47 gene.

In some embodiments, the targeting vector further includes a selectable gene marker. In some embodiments, the target region is derived from human. In some embodiments, the target region is a part or entirety of the nucleotide sequence of a humanized CD47. In some embodiments, the nucleotide sequence is shown as one or more of exon 1, exon 2, exon 3, exon 4, exon 5, exon 6, exon 7, exon 8, exon 9, exon 10, and exon 11 of the human CD47.

In some embodiments, the nucleotide sequence of the human CD47 encodes the human CD47 protein with the NCBI accession number NP_001768.1 (SEQ ID NO: 81). In some emboldens, the nucleotide sequence of the human CD47 is selected from the nucleotides from the position 108080013 to the position 108080324 of NC_000003.12 with T→C point mutation at 108080196.

The disclosure also relates to a cell including the targeting vector as described herein.

The disclosure also relates to a method for establishing a genetically-modified non-human animal expressing two human or chimeric (e.g., humanized) genes. The method includes the steps of

(a) using the method for establishing a CD47 gene humanized animal model to obtain a CD47 gene genetically modified humanized mouse;

(b) mating the CD47 gene genetically modified humanized mouse obtained in step (a) with another humanized mouse, and then screening to obtain a double humanized mouse model.

In some embodiments, in step (b), the CD47 gene genetically modified humanized mouse obtained in step (a) is mated with a SIRPα humanized mouse to obtain a CD47 and SIRPα double humanized mouse model.

The disclosure also relates to non-human mammal generated through the methods as described herein.

In some embodiments, the genome thereof contains human gene(s).

In some embodiments, the non-human mammal is a rodent. In some embodiments, the non-human mammal is a mouse.

In some embodiments, the non-human mammal expresses a protein encoded by a humanized CD47 gene.

The disclosure also relates to an offspring of the non-human mammal.

In another aspect, the disclosure relates to a tumor bearing non-human mammal model, characterized in that the non-human mammal model is obtained through the methods as described herein.

In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to a CD47 genomic DNA sequence of a humanized mouse, a DNA sequence obtained by a reverse transcription of the mRNA obtained by transcription thereof is consistent with or complementary to the DNA sequence; a construct expressing the amino acid sequence thereof a cell comprising the construct thereof; a tissue comprising the cell thereof.

This disclosure is related to genetically modified animals that have a disruption at the endogenous CD132 gene (e.g., CD132 knockout), and methods of making and use thereof.

In one aspect, the disclosure relates to a genetically-modified, non-human animal whose genome comprise a disruption in the animal's endogenous CD132 gene, wherein the disruption of the endogenous CD132 gene comprises deletion of exon 2 of the endogenous CD132 gene.

In some embodiments, the disruption of the endogenous CD132 gene further comprises deletion of exon 1 of the endogenous CD132 gene. In some embodiments, the disruption of the endogenous CD132 gene comprises deletion of part of exon 1 of the endogenous CD132 gene.

In some embodiments, the disruption of the endogenous CD132 gene further comprises deletion of one or more exons or part of exons selected from the group consisting of exon 3, exon 4, exon 5, exon 6, exon 7, and exon 8 of the endogenous CD132 gene. In some embodiments, the disruption of the endogenous CD132 gene comprises deletion of exons 1-8 of the endogenous CD132 gene.

In some embodiments, the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene.

In some embodiments, the disruption consists of deletion of more than 150 nucleotides in exon 1; deletion of the entirety of intron 1, exon 2, intron 2, exon 3, intron 3, exon 4, intron 4, exon 5, intron 5, exon 6, intron 6, exon 7, intron 7; and deletion of more than 250 nucleotides in exon 8.

In some embodiments, the animal is homozygous with respect to the disruption of the endogenous CD132 gene. In some embodiments, the animal is heterozygous with respect to the disruption of the endogenous CD132 gene.

In some embodiments, the disruption prevents the expression of functional CD132 protein.

In some embodiments, the length of the remaining exon sequences at the endogenous CD132 gene locus is less than 30% of the total length of all exon sequences of the endogenous CD132 gene. In some embodiments, the length of the remaining sequences at that the endogenous CD132 gene locus is less than 15% of the full sequence of the endogenous CD132 gene.

In another aspect, the disclosure relates to a genetically-modified, non-human animal, wherein the genome of the animal does not have exon 2 of CD132 gene at the animal's endogenous CD132 gene locus.

In some embodiments, the genome of the animal does not have one or more exons or part of exons selected from the group consisting of exon 1, exon 3, exon 4, exon 5, exon 6, exon 7, and exon 8. In some embodiments, the genome of the animal does not have one or more introns or part of introns selected from the group consisting of intron 1, intron 2, intron 3, intron 4, intron 5, intron 6, and intron 7.

In one aspect, the disclosure also provides a CD132 knockout non-human animal, wherein the genome of the animal comprises from 5′ to 3′ at the endogenous CD132 gene locus, (a) a first DNA sequence; optionally (b) a second DNA sequence comprising an exogenous sequence; (c) a third DNA sequence, wherein the first DNA sequence, the optional second DNA sequence, and the third DNA sequence are linked, wherein the first DNA sequence comprises an endogenous CD132 gene sequence that is located upstream of intron 1, the second DNA sequence can have a length of 0 nucleotides to 300 nucleotides, and the third DNA sequence comprises an endogenous CD132 gene sequence that is located downstream of intron 7.

In some embodiments, the first DNA sequence comprises a sequence that has a length (5′ to 3′) of from 10 to 100 nucleotides (e.g., approximately 10, 20, 30, 40, 50, 60, 70, 80, 90, 100 nucleotides), wherein the length of the sequence refers to the length from the first nucleotide in exon 1 of the CD132 gene to the last nucleotide of the first DNA sequence.

In some embodiments, the first DNA sequence comprises at least 10 nucleotides from exon 1 of the endogenous CD132 gene. In some embodiments, the first DNA sequence has at most 100 nucleotides from exon 1 of the endogenous CD132 gene.

In some embodiments, the third DNA sequence comprises a sequence that has a length (5′ to 3′) of from 200 to 600 nucleotides (e.g., approximately 200, 250, 300, 350, 400, 450, 500, 550, 600 nucleotides), wherein the length of the sequence refers to the length from the first nucleotide in the third DNA sequence to the last nucleotide in exon 8 of the endogenous CD132 gene.

In some embodiments, the third DNA sequence comprises at least 300 nucleotides from exon 8 of the endogenous CD132 gene. In some embodiments, the third DNA sequence has at most 400 nucleotides from exon 8 of the endogenous CD132 gene.

In one aspect, the disclosure also relates to a genetically-modified, non-human animal produced by a method comprising knocking out one or more exons of endogenous CD132 gene by using (1) a first nuclease comprising a zinc finger protein, a TAL-effector domain, or a single guide RNA (sgRNA) DNA-binding domain that binds to a target sequence in exon 1 of the endogenous CD132 gene or upstream of exon 1 of the endogenous CD132 gene, and (2) a second nuclease comprising a zinc finger protein, a TAL-effector domain, or a single guide RNA (sgRNA) DNA-binding domain that binds to a sequence in exon 8 of the endogenous CD132 gene.

In some embodiments, the nuclease is CRISPR associated protein 9 (Cas9). In some embodiments, the target sequence in exon 1 of the endogenous CD132 gene or upstream of exon 1 of the endogenous CD132 gene is set forth in SEQ ID NO: 1, 2, 3, or 4, and the target sequence in exon 8 of the endogenous CD132 gene is set forth in SEQ ID NO: 5, 6, 7, or 8. In some embodiments, the first nuclease comprises a sgRNA that targets SEQ ID NO: 3 and the second nuclease comprises a sgRNA that targets SEQ ID NO: 6.

In some embodiments, the animal does not express a functional CD132 protein. In some embodiments, the animal does not express a functional interleukin-2 receptor.

In some embodiments, the animal has one or more of the following characteristics:
(a) the percentage of T cells (CD3+ cells) is less than 2%, 1.5%, 1%, 0.7%, or 0.5% of leukocytes in the animal;
(b) the percentage of B cells (e.g., CD3− CD19+ cells) is less than 0.1% or 0.05% of leukocytes in the animal;
(c) the percentage of NK cells (e.g., CD3− CD49b+ cells) is less than 2% or 1.5% of leukocytes in the animal;
(d) the percentage of CD4+ T cells is less than 0.5%, 0.3%, or 0.1% of T cells;

(e) the percentage of CD8+ T cells is less than 0.5%, 0.3%, or 0.1% of T cells;
(f) the percentage of CD3+CD4+ cells, CD3+CD8+ cells, CD3− CD19+ cells is less than 1% or 0.5% of leukocytes in the animal;
(g) the percentage of T cells, B cells, and NK cells is less than 5%, 4%, 3%, 2% or 1% of leukocytes in the animal.

In some embodiments, the animal after being engrafted with human hematopoietic stem cells to develop a human immune system has one or more of the following characteristics:
(a) the percentage of human CD45+ cells is greater than 70% or 80% of leukocytes of the animal;
(b) the percentage of human CD3+ cells is greater than 45% of leukocytes in the animal; and
(c) the percentage of human CD19+ cells is greater than 20% or 25% of leukocytes in the animal.

In some embodiments, the animal has an enhanced engraftment capacity of exogenous cells relative to a NOD/scid mouse.

In some embodiments, the animal has one or more of the following characteristics:
(a) the animal has no functional T-cells and/or no functional B-cells;
(b) the animal exhibits reduced macrophage function relative to a NOD/scid mouse;
(c) the animal exhibits no NK cell activity; and
(d) the animal exhibits reduced dendritic function relative to a NOD/scid mouse.

In some embodiments, the animal is a mammal, e.g., a monkey, a rodent, a rat, or a mouse. In some embodiments, the animal is a NOD/scid mouse, or a NOD/scid nude mouse.

In some embodiments, the animal further comprises a sequence encoding a human or chimeric protein. In some embodiments, the human or chimeric protein is programmed cell death protein 1 (PD-1) or CD137.

In some embodiments, the animal further comprises a disruption in the animal's endogenous Beta-2-Microglobulin (B2m) gene and/or a disruption in the animal's endogenous Forkhead Box N1 (Foxn1) gene.

In another aspect, the disclosure also relates to methods of determining effectiveness of an agent or a combination of agents for the treatment of cancer. The methods involve the steps of engrafting tumor cells to the animal as described herein, thereby forming one or more tumors in the animal; administering the agent or the combination of agents to the animal; and determining the inhibitory effects on the tumors.

In some embodiments, before engrafting the tumor cells to the animal, human peripheral blood cells (hPBMC) or human hematopoietic stem cells are injected to the animal.

In some embodiments, the tumor cells are from cancer cell lines. In some embodiments, the tumor cells are from a tumor sample obtained from a human patient.

In some embodiments, the inhibitory effects are determined by measuring the tumor volume in the animal.

In some embodiments, the tumor cells are melanoma cells, lung cancer cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

In some embodiments, the agent is an anti-CD47 antibody or an anti-PD-1 antibody.

In some embodiments, the combination of agents comprises one or more agents selected from the group consisting of paclitaxel, cisplatin, carboplatin, pemetrexed, 5-FU, gemcitabine, oxaliplatin, docetaxel, and capecitabine.

In one aspect, the disclosure is also related to methods of producing a CD132 knockout mouse. The methods involve
(a) transforming a mouse embryonic stem cell with a gene editing system that targets endogenous CD132 gene, thereby producing a transformed embryonic stem cell;
(b) introducing the transformed embryonic stem cell into a mouse blastocyst;
(c) implanting the mouse blastocyst into a pseudopregnant female mouse; and
(d) allowing the blastocyst to undergo fetal development to term, thereby obtaining the CD132 knockout mouse.

In another aspect, the disclosure also provides methods of producing a CD132 knockout mouse. The methods include the steps of
(a) transforming a mouse embryonic stem cell with a gene editing system that targets endogenous CD132 gene, thereby producing a transformed embryonic stem cell;
(b) implanting the transformed embryonic cell into a pseudopregnant female mouse; and
(c) allowing the transformed embryonic cell to undergo fetal development to term, thereby obtaining the CD132 knockout mouse.

In some embodiments, the gene editing system comprises a first nuclease comprising a zinc finger protein, a TAL-effector domain, or a single guide RNA (sgRNA) DNA-binding domain that binds to a target sequence in exon 1 of the endogenous CD132 gene or upstream of exon 1 of the endogenous CD132 gene, and a second nuclease comprising a zinc finger protein, a TAL-effector domain, or a single guide RNA (sgRNA) DNA-binding domain that binds to a sequence in exon 8 of the endogenous CD132 gene.

In some embodiments, the nuclease is CRISPR associated protein 9 (Cas9).

In some embodiments, the target sequence in exon 1 of the endogenous CD132 gene or upstream of exon 1 of the endogenous CD132 gene is set forth in SEQ ID NO: 1, 2, 3, or 4, and the target sequence in exon 8 of the endogenous CD132 gene is set forth in SEQ ID NO: 5, 6, 7, or 8.

In some embodiments, the mouse embryonic stem cell has a NOD/scid background, or a NOD/scid nude background.

In some embodiments, the mouse embryonic stem cell comprises a sequence encoding a human or chimeric protein. In some embodiments, the human or chimeric protein is PD-1 or CD137.

In some embodiments, the mouse embryonic stem cell has a genome comprising a disruption in the animal's endogenous Beta-2-Microglobulin (B2m) gene and/or a disruption in the animal's endogenous Forkhead Box N1 (Foxn1) gene.

In another aspect, the disclosure relates to a non-human mammalian cell, comprising a disruption, a deletion, or a genetic modification as described herein.

In some embodiments, the cell includes Cas9 mRNA or an in vitro transcript thereof.

In some embodiments, the non-human mammalian cell is a mouse cell. In some embodiments, the cell is a fertilized egg cell. In some embodiments, the cell is a germ cell. In some embodiments, the cell is a blastocyst.

In another aspect, the disclosure relates to methods for establishing a CD132 knockout animal model. The methods include the steps of:
(a) providing the cell with a disruption in the endogenous CD132 gene, and preferably the cell is a fertilized egg cell;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring of the pregnant female in step (c).

In some embodiments, the establishment of a CD132 knockout animal involves a gene editing technique that is based on CRISPR/Cas9.

In some embodiments, the non-human mammal is mouse. In some embodiments, the non-human mammal in step (c) is a female with false pregnancy.

In another aspect, the disclosure relates to a tumor bearing non-human mammal model, characterized in that the non-human mammal model is obtained through the methods as described herein.

The disclosure also relates to a cell or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the CD132 gene function, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing sgRNA activity testing results. PC− is a negative control. PC+ is a positive control. L− is a control for 5′ end targeting sites. L1 to L4 correspond to sgRNA-1 to sgRNA-4. R− is a control for 3′ end targeting sites. R5-R8 correspond to sgRNA-5 to sgRNA8.

FIG. 2 shows PCR results of B-NDG mouse tail genomic DNA. Primers PCR-1F and PCR-1R were used. M is a marker. WT is a NOD/scid control. “-” is a water control. 1-4 are mouse numbers.

FIG. 3A shows a schematic diagram of mouse SIRPα gene.

FIG. 3B shows a schematic diagram of human SIRPα gene.

FIG. 4 is a schematic diagram showing SIRPα gene targeting strategy.

FIG. 5 shows the restriction enzymes digestion results of the plasmid pClon-4G-SIRPα. The numbers 1-6 indicate six different pClon-4G-SIRPα clones. M is a marker. ck indicates control plasmid without restriction enzyme digestion.

FIG. 6 is a graph showing sgRNA activity testing results. Con is a negative control. PC is a positive control.

FIG. 7 shows a schematic diagram of the humanized SIRPα gene.

FIGS. 8A-8B show PCR identification results of samples collected from tails of F0 generation mice using primers L-GT-F and L-GT-R. M is a marker. H₂O is a water control. F0-1, F0-2, F0-3, and F0-4 are mouse numbers.

FIG. 8C shows PCR identification results of samples collected from tails of F0 generation mice using primers R-GT-F and R-GT-R. WT is wildtype. M is a marker. PC1 and PC2 are positive controls. H₂O is a water control. F0-1, F0-2, F0-3, and F0-4 are mouse numbers.

FIG. 9A shows PCR identification results of samples collected from tails of F1 generation mice using primers L-GT-F and L-GT-R. WT is wildtype. M is a marker. PC1 and PC2 are positive controls. H₂O is a water control. F1-1, F1-2, and F1-3 are mouse numbers.

FIG. 9B shows PCR identification results of samples collected from tails of F1 generation mice using primers R-GT-F and R-GT-R. WT is wildtype. M is a marker. PC1 and

PC2 are positive controls. H₂O is a water control. F1-1, F1-2, and F1-3 are mouse numbers.

FIG. 10 shows Southern blot results for F1 generation mice. WT is wildtype. F1-1, F1-2, and F1-3 are mouse numbers.

FIG. 11A shows RT-PCR identification results of B-NDG mice (+/+) and humanized SIRPα homozygous mice (H/H) using primers mSIRPA-F1 and mSIRPA-R1. H₂O is a negative control.

FIG. 11B shows RT-PCR identification results of B-NDG mice (+1+) and humanized SIRPα homozygous mice (H/H) using primers mSIRPA-F2 and mSIRPA-R2. H₂O is a negative control.

FIG. 11C shows RT-PCR identification results of B-NDG mice (+/+) and humanized SIRPα homozygous mice (H/H) using primers hSIRPA-F1 and hSIRPA-R1. H₂O is a negative control.

FIG. 11D shows RT-PCR identification results of B-NDG mice (+/+) and humanized SIRPα homozygous mice (H/H) using primers hSIRPA-F2 and hSIRPA-R2. H₂O is a negative control.

FIG. 11E shows RT-PCR identification results of B-NDG mice (+1+) and humanized SIRPα homozygous mice (H/H) using primers GAPDH-F and GAPDH-R. H₂O is a negative control.

FIG. 12A is a flow cytometry result of spleen cells from B-NDG mice (+/+).

FIG. 12B is a flow cytometry result of spleen cells from B-NDG_hSIRPα mice (H/H).

FIG. 12C is a flow cytometry result of spleen cells from B-NDG mice (+/+).

FIG. 12D is a flow cytometry result of spleen cells from B-NDG_hSIRPα mice (H/H).

FIG. 13A is a flow cytometry result of cells within abdominal cavity washing fluid from B-NDG mice (+/+).

FIG. 13B is a flow cytometry result of cells within abdominal cavity washing fluid from B-NDG_hSIRPα mice (H/H).

FIG. 13C is a flow cytometry result of cells within abdominal cavity washing fluid from B-NDG mice (+/+).

FIG. 13D is a flow cytometry result of cells within abdominal cavity washing fluid from B-NDG_hSIRPα mice (H/H).

FIG. 14 shows percentages of granulocytes, dendritic cells (DC), and monocytes/macrophages of CD45+ leukocytes in mouse spleen. Either B-NDG mice or B-NDG_hSIRPα mice were selected for detection.

FIG. 15 is a schematic diagram showing CD47 gene targeting strategy.

FIG. 16 shows percentages of differentiated immune cell subsets in blood CD45+ cells of C57BL/6 mice, B-NDG mice and B-NDG_hSIRPα/hCD47 mice.

FIG. 17 shows body weight of B-NDG_hSIRPα/hCD47 mice injected with B-luciferase-GFP Raji cells and administered with PBS (G1), Rituximab (G2), KWAR23 (G3), or Rituximab+KWAR23 (G4).

FIG. 18 shows body weight change of B-NDG_hSIRPα/hCD47 mice injected with B-luciferase-GFP Raji cells and administered with PBS (G1), Rituximab (G2), KWAR23 (G3), or Rituximab+KWAR23 (G4).

FIG. 19 shows survival curves of B-NDG_hSIRPα/hCD47 mice injected with B-luciferase-GFP Raji cells and administered with PBS (G1), Rituximab (G2), KWAR23 (G3), or

Rituximab+KWAR23 (G4).

FIG. 20 shows tumor fluorescence image of B-NDG_hSIRPα/hCD47 mice injected with B-luciferase-GFP Raji cells and administered with PBS (G1), Rituximab (G2), KWAR23 (G3), or Rituximab+KWAR23 (G4). D7, D14, and D24 indicate 7, 14, and 24 days after grouping, respectively.

FIG. 21 shows the alignment between mouse SIRPα amino acid sequence (NP_031573.2; SEQ ID NO: 79) and human SIRPα amino acid sequence (NP 542970.1; SEQ ID NO: 77).

FIG. 22 shows the alignment between mouse CD47 amino acid sequence (NP_034711.1; SEQ ID NO: 83) and human CD47 amino acid sequence (NP_001768.1; SEQ ID NO: 81).

### DETAILED DESCRIPTION

This disclosure relates to transgenic non-human animal with human or chimeric (e.g., humanized) SIRPα/CD47, and methods of use thereof.

Signal regulatory protein α (SIRPα) is a regulatory membrane glycoprotein from SIRP family. SIRPα acts as inhibitory receptor and interacts with a broadly expressed transmembrane protein CD47. This interaction negatively controls effector function of innate immune cells such as host cell phagocytosis. SIRPα diffuses laterally on the macrophage membrane and accumulates at a phagocytic synapse to bind CD47, which inhibits the cytoskeleton-intensive process of phagocytosis by the macrophage. CD47 provides a “do not eat” signal by binding to the N-terminus of signal regulatory protein alpha (SIRPα). It has been found to be overexpressed in many different tumor cells. Thus, targeting CD47 and/or SIRPα is in the spotlight of cancer immunotherapy. Blocking CD47 or SIRPα triggers the recognition and elimination of cancer cells by the innate immunity. These antibodies or binding agents that target CD47 or SIRPα can be used to treat various tumors and cancers, e.g., solid tumors, hematologic malignancies (e.g., relapsed or refractory hematologic malignancies), acute myeloid leukemia, non-Hodgkin's lymphoma, breast cancer, bladder cancer, ovarian cancer, and small cell lung cancer tumors. The anti-CD47 or anti-SIRPα antibodies are described, e.g., in Huang et al. “Targeting CD47: the achievements and concerns of current studies on cancer immunotherapy.” Journal of thoracic disease 9.2 (2017): E168; Liu et al. “Pre-clinical development of a humanized anti-CD47 antibody with anti-cancer therapeutic potential.” PloS one 10.9 (2015): e0137345; Ansell et al. “A phase 1 study of TTI-621, a novel immune checkpoint inhibitor targeting CD47, in patients with relapsed or refractory hematologic malignancies.” (2016): 1812-1812; Yanagita et al. “Anti-SIRPα antibodies as a potential new tool for cancer immunotherapy.” JCI insight 2.1 (2017); each of which is incorporated herein by reference in its entirety.

Experimental animal models are an indispensable research tool for studying the effects of these antibodies. Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. The genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels. Furthermore, because of interaction between human SIRPα and human CD47, a desirable animal model for the investigation of anti-SIRPα or anti-CD47 antibodies should faithfully mimic the interaction between human SIRPα and human CD47, elicit robust responses from both the innate and adaptive immunity, and recapitulate side effects of CD47 blockade on RBCs and platelets (Huang et al. “Targeting CD47: the achievements and concerns of current studies on cancer immunotherapy.” Journal of thoracic disease 9.2 (2017): E168).

However, these humanized CD47/SIRPα animal models are not suitable for testing the phagocytosis effects of these antibodies as many of them may induce antibody-dependent cellular cytotoxicity (ADCC) or some other immune responses. Those effects can skew the results and make it difficult to evaluate phagocytosis. The genetically modified animals as described herein do not have functional T cells or B cells, but they still have functional phagocytic cells, e.g., neutrophils, eosinophils (acidophilus), basophils, or monocytes. Macrophages can be derived from monocytes, and can engulf and digest cellular debris, foreign substances, microbes, cancer cells. Thus, the genetically modified animals described herein can be used to determine the effect of an agent (e.g., anti-CD47 antibodies or anti-SIRPα antibodies) on phagocytosis, and are particularly useful for determining the effects of the agent to inhibit the growth of tumor cells. Therefore, the genetically modified animals with humanized SIRPα and/or CD47 as described herein provide an advantageous model to test the effects of anti-hCD47 antibodies or anti-hSIRPα antibodies on phagocytosis. Furthermore, the genetically-modified animals described herein can be injected with human PBMC or CD34+ cells for immune system reconstitution. As functional endogenous macrophages and granulocytes remain in B-NDG mice, B-NDG mice with humanized CD47/SIRPα gene after immune system reconstitution provides an immune system that is even more similar to that of human, which is particularly useful for testing agents that target human immune system.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U.S. Pat. No. 4,683,195; Nucleic Acid Hybridization (B. D. Hames& S. J. Higgins eds. 1984); Transcription And Translation (B. D. Hames & S. J. Higgins eds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### Humanized SIRPα Animal

The present disclosure provides genetically-modified immune-deficient animal whose genome comprises at least one chromosome comprising a sequence encoding a human or chimeric SIRPα. Signal regulatory protein a (SIRPα, SIRPα, Sirpa, SIRPA, or CD172A) is a transmembrane protein. It has an extracellular region comprising three Ig-like domains and a cytoplasmic region containing immunoreceptor tyrosine-based inhibition motifs that mediate binding of the protein tyrosine phosphatases SHP1 and SHP2. Tyrosine phosphorylation of SIRPα is regulated by various growth factors and cytokines as well as by integrin-mediated cell adhesion to extracellular matrix proteins. SIRPα is especially abundant in myeloid cells such as macrophages and dendritic cells, whereas it is expressed at only low levels in T, B, NK, and NKT cells.

The extracellular region of SIRPα can interact with its ligand CD47. The interaction of SIRPα on macrophages with CD47 on red blood cells prevents phagocytosis of Ig-opsonized red blood cells by macrophages in vitro and in vivo. The ligation of SIRPα on phagocytes by CD47 expressed on a neighboring cell results in phosphorylation of SIRPα cytoplasmic immunoreceptor tyrosine-based inhibition (ITIM) motifs, leading to the recruitment of SHP-1 and SHP-2 phosphatases. One resulting downstream effect is the prevention of myosin-IIA accumulation at the phagocytic synapse and consequently inhibition of phagocytosis. Thus, CD47-SIRPα interaction functions as a negative immune checkpoint to send a “don't eat me” signal to ensure that healthy autologous cells are not inappropriately phagocytosed. However, overexpression of CD47 has also been found in nearly all types of tumors, some of which include acute myeloid leukemia, non-Hodgkin's lymphoma, bladder cancer, and breast cancer. Such negative regulation of macrophages can be minimized by blocking the binding of CD47 to SIRPα. Thus, antibodies against CD47 or SIRPα can promote both Ab-dependent cellular phagocytosis (ADCP) and in some cases, trigger Ab-dependent cellular cytotoxicity (ADCC), thus can be used to treat various cancers.

The extracellular region of SIRPα interacts with its ligand, CD47, through an IgV-like domain at the NH2-terminus of the extracellular region of SIRPα.

A detailed description of SIRPα and its function can be found, e.g., in Murata, Y. et al., “The CD47—SIRPα signalling system: its physiological roles and therapeutic application.” The Journal of Biochemistry 155.6 (2014): 335-344; Yanagita et al. “Anti-SIRPα antibodies as a potential new tool for cancer immunotherapy.” JCI insight 2.1 (2017); Seiffert et al. “Signal-regulatory protein α (SIRPα) but not SIRPβ is involved in T-cell activation, binds to CD47 with high affinity, and is expressed on immature CD34+CD38− hematopoietic cells.” Blood 97.9 (2001): 2741-2749; which are incorporated by reference herein in the entirety.

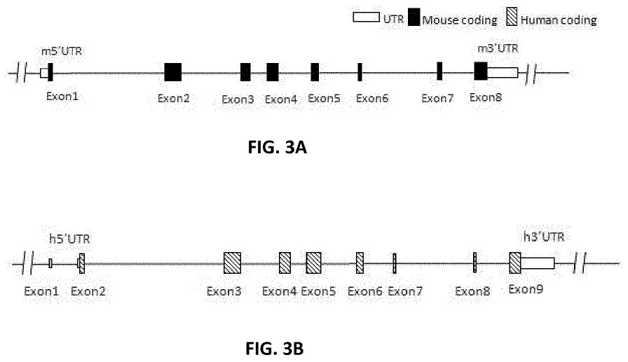

In human genomes, SIRPα gene (Gene ID: 140885) locus has 9 exons, exon 1, exon 2, exon 3, exon 4, exon 5, exon 6, exon 7, exon 8, and exon 9 (FIG. 3B). The SIRPα protein has an extracellular region, a transmembrane region, and a cytoplasmic region. The signal peptide is located at the extracellular region. The nucleotide sequence for human SIRPα mRNA is NM_080792.2 (SEQ ID NO: 76), and the amino acid sequence for human SIRPα is NP_542970.1 (SEQ ID NO: 77). The location for each exon and each region in human SIRPα nucleotide sequence and amino acid sequence is listed below:

TABLE 1

| | NM_080792.2 | NP_542970.1 |
|---|---|---|
| Human SIRPα | 3868 bp | 504 aa |
| (approximate location) | (SEQ ID NO: 76) | (SEQ ID NO: 77) |
| Exon 1 | 1-18 | Non-coding range |
| Exon 2 | 19-106 | 1-26 |
| Exon 3 | 107-463 | 27-145 |
| Exon 4 | 464-781 | 146-251 |
| Exon 5 | 782-1114 | 252-362 |
| Exon 6 | 1115-1228 | 363-400 |
| Exon 7 | 1229-1253 | 401-409 |
| Exon 8 | 1254-1293 | 410-422 |
| Exon 9 | 1294-3868 | 423-504 |
| Signal peptide | 28-117 | 1-30 |
| Extracellular region | 118-1146 | 31-373 |
| (excluding signal | | |
| peptide region) | | |
| Transmembrane region | 1147-1209 | 374-394 |
| Cytoplasmic region | 1210-1539 | 395-504 |
| Donor in one example | 118-441 | 31-138 |

The extracellular region of human SIRPα comprises an Ig-like V-type domain and two Ig-like C1-type domains. According to the information of human SIRPα from the UniProt Database (UniProt identifier: P78324), the Ig-like V-type domain, the Ig-like C1-type 1 domain, and the Ig-like C1-type 2 domain correspond to amino acids 32-137, amino acids 148-247, and amino acids 254-348 of the human SIRPα protein (SEQ ID NO: 77; or NP_542970.1). Specifically, the Ig-like V-type domain, the Ig-like C1-type 1 domain, and the Ig-like C1-type 2 domain are encoded by a portion of exon 3, a portion of exon 4, and a portion of exon 5 of human SIRPα (SEQ ID NO: 77). Thus, the donor region is largely within the extracellular Ig-like V-type domain (N-terminal IgV domain).

Human SIRPα also have several transcript variants. These variants can also be used to make humanized animals, and they are summarized below.

TABLE 2

| Human SIRPα transcript variants | Amino acid sequences |
|---|---|
| NM_001040022.1 | NP_001035111.1 |
| NM_001040023.1 | NP_001035112.1 |
| NM_001330728.1 | NP_001317657.1 |
| XM_005260670.3 | XP_005260727.1 |
| XM_006723545.3 | XP_006723608.1 |
| XM_011529173.2 | XP_011527475.1 |

In mice, SIRPα gene locus has 8 exons, exon 1, exon 2, exon 3, exon 4, exon 5, exon 6, exon 7, and exon 8 (FIG. 3A). The mouse SIRPα protein also has an extracellular region, a transmembrane region, and a cytoplasmic region, and the signal peptide is located at the extracellular region. The nucleotide sequence for mouse SIRPα mRNA is NM_007547.4 (SEQ ID NO: 78), the amino acid sequence for mouse SIRPα is NP_031573.2 (SEQ ID NO: 79). The location for each exon and each region in the mouse SIRPα nucleotide sequence and amino acid sequence is listed below:

TABLE 3

| | NM_007547.4 | NP_031573.2 |
|---|---|---|
| Mouse SIRPα | 4031 bp | 509aa |
| (approximate location) | (SEQ ID NO: 78) | (SEQ ID NO: 79) |
| Exon 1 | 1-526 | 1-27 |
| Exon 2 | 527-883 | 28-146 |
| Exon 3 | 884-1201 | 147-252 |
| Exon 4 | 1202-1537 | 253-364 |
| Exon 5 | 1538-1651 | 365-402 |
| Exon 6 | 1652-1676 | 403-411 |
| Exon 7 | 1677-1716 | 412-424 |
| Exon 8 | 1717-4018 | 425-509 |
| Signal peptide | 445-537 | 1-31 |
| Extracellular region | 538-1557 | 32-371 |
| (excluding signal peptide region) | | |
| Transmembrane region | 1558-1632 | 372-396 |
| Cytoplasmic region | 1633-1971 | 397-509 |
| Replaced region in one example | 538-861 | 32-139 |

The mouse SIRPα gene (Gene ID: 19261) is located in Chromosome 2 of the mouse genome, which is located from 129592665 to 129632228, of NC_000068.7 (GRCm38.p4 (GCF_000001635.24)). The 5′-UTR is from 129,593,205 to 129,593,612, exon 1 is from 129,593,205 to 129,593,694, the first intron is from 129,593,695 to 129,608,903, exon 2 is from 129,608,904 to 129,609,260, the second intron is from 129,609,261 to 129,615,446, exon 3 is from 129,615,447 to 129,615,764, the third intron is from 129,615,765 to 129,616,222, exon 4 is from 129,616,223 to 129,616,558, the fourth intron is from 129,616,559 to 129,618,456, exon 5 is from 129,618,457 to 129,618,570, the fifth intron is from 129,618,571 to 129,621,202, exon 6 is from 129,621,203 to 129,621,227, the sixth intron is from 129,621,228 to 129,627,945, exon 7 is from 129,627,946 to 129,627,985, the seventh intron is from 129,627,986 to 129,629,926, exon 8 is from 129,629,927 to 129,632,228, the 3′-UTR is from 129,630,185 to 129,632,228, base on transcript NM_007547.4.

Thus, exon 1 in mouse SIRPα roughly corresponds to exon 2 in human SIRPα, exon 2 in mouse SIRPα roughly corresponds to exon 3 in human SIRPα, exon 3 in mouse SIRPα roughly corresponds to exon 4 in human SIRPα, exon 4 in mouse SIRPα roughly corresponds to exon 5 in human SIRPα, exon 5 in mouse SIRPα roughly corresponds to exon 6 in human SIRPα, exon 6 in mouse SIRPα roughly corresponds to exon 7 in human SIRPα, exon 7 in mouse SIRPα roughly corresponds to exon 8 in human SIRPα, and exon 8 in mouse SIRPα roughly corresponds to exon 9 in human SIRPα.

All relevant information for mouse SIRPα locus can be found in the NCBI website with Gene ID: 19261, which is incorporated by reference herein in its entirety.

The mouse SIRPα has several transcript variants. A portion of these sequences can also be replaced by corresponding human sequences. These variants are summarized in Table 4.

TABLE 4

| Mouse SIRPα transcript variants | Amino acid sequences |
|---|---|
| NM_001177647.2 | NP_001171118.1 |
| NM_001291019.1 | NP_001277948.1 |
| NM_001291020.1 | NP_001277949.1 |
| NM_001291021.1 | NP_001277950.1 |

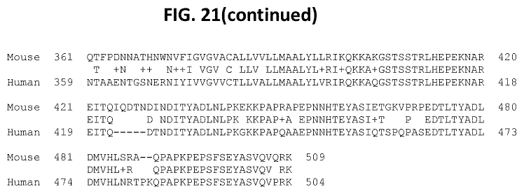

FIG. 21 shows the alignment between mouse SIRPα amino acid sequence (NP_031573.2; SEQ ID NO: 79) and human SIRPα amino acid sequence (NP_542970.1; SEQ ID NO: 77). Thus, the corresponding amino acid residue or region between human and mouse SIRPα can also be found in FIG. 21.

SIRPα genes, proteins, and locus of the other species are also known in the art. For example, the gene ID for SIRPα in Rattus norvegicus is 25528, the gene ID for SIRPα in Macaca mulatta (Rhesus monkey) is 717811, the gene ID for SIRPα in Canis lupus familiaris (dog) is 609452, and the gene ID for SIRPα in Sus scrofa (pig) is 494566. The relevant information for these genes (e.g., intron sequences, exon sequences, amino acid residues of these proteins) can be found, e.g., in NCBI database.

The present disclosure provides human or chimeric (e.g., humanized) SIRPα nucleotide sequence and/or amino acid sequences. In some embodiments, the entire sequence of mouse exon 1, exon 2, exon 3, exon 4, exon 5, exon 6, exon 7, exon 8, the signal peptide, the extracellular region, the transmembrane region, and/or the cytoplasmic region are replaced by the corresponding human sequence.

In some embodiments, a “region” or “portion” of mouse exon 1, exon 2, exon 3, exon 4, exon 5, exon 6, exon 7, exon 8, signal peptide, the extracellular region, the transmembrane region, and/or the cytoplasmic region is replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 150, 200, 250, 300, 350, or 400 nucleotides, or at least 1, 2, 3, 4, 5, 6, 7, 8, 9, 10, 20, 30, 40, 50, 60, 70, 80, 90, 100, 110, 120, 130, or 150 amino acid residues.

In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, signal peptide, the extracellular region, the transmembrane region, and/or the cytoplasmic region. In some embodiments, a region, a portion, or the entire sequence of mouse exon 2 is replaced by a region, a portion, or the entire sequence of human exon 3.

In some embodiments, a “region” or “portion” of mouse exon 1, exon 2, exon 3, exon 4, exon 5, exon 6, exon 7, exon 8, signal peptide, the extracellular region, the transmembrane region, and/or the cytoplasmic region is deleted.

Thus, in some embodiments, the present disclosure also provides a chimeric (e.g., humanized) SIRPα nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse SIRPα mRNA sequence (e.g., SEQ ID NO: 78), mouse SIRPα amino acid sequence (e.g., SEQ ID NO: 79), or a portion thereof (e.g., exon 1, exon 2, exon 3, exon 4, exon 5, exon 6, exon 7, or exon 8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human SIRPα mRNA sequence (e.g., SEQ ID NO: 76), human SIRPα amino acid sequence (e.g., SEQ ID NO: 77), or a portion thereof (e.g., exon 1, exon 2, exon 3, exon 4, exon 5, exon 6, exon 7, exon 8, or exon 9).

In some embodiments, a sequence encoding amino acids 32-139 of mouse SIRPα (SEQ ID NO: 79) is replaced. In some embodiments, the sequence is replaced by a sequence encoding a corresponding region of human SIRPα (e.g., amino acids 31-138 of human SIRPα (SEQ ID NO: 77).

In some embodiments, a sequence encoding the entire of part of the extracellular Ig-like V-type domain of mouse SIRPα (SEQ ID NO: 79) is replaced. In some embodiments, the sequence is replaced by a sequence encoding a corresponding region (e.g., the entire or part of the extracellular Ig-like V-type domain) of human SIRPα (SEQ ID NO: 77). In some embodiments, the animal has a humanized Ig-like V-type domain. In some embodiments, the animal has a humanized Ig-like C1-type 1 domain and/or a humanized Ig-like C1-type 2 domain.

In some embodiments, the nucleic acids as described herein are operably linked to a promotor or regulatory element, e.g., an endogenous mouse SIRPα promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse SIRPα nucleotide sequence (e.g., exon 1, exon 2, exon 3, exon 4, exon 5, exon 6, exon 7, exon 8, or SEQ ID NO: 78).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse SIRPα nucleotide sequence (e.g., exon 1, exon 2, exon 3, exon 4, exon 5, exon 6, exon 7, exon 8, or SEQ ID NO: 78).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human SIRPα nucleotide sequence (e.g., exon 1, exon 2, exon 3, exon 4, exon 5, exon 6, exon 7, exon 8, exon 9, or SEQ ID NO: 76).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human SIRPα nucleotide sequence (e.g., exon 1, exon 2, exon 3, exon 4, exon 5, exon 6, exon 7, exon 8, exon 9, or SEQ ID NO: 76).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse SIRPα amino acid sequence (e.g., exon 1, exon 2, exon 3, exon 4, exon 5, exon 6, exon 7, exon 8, or SEQ ID NO: 79).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse SIRPα amino acid sequence (e.g., exon 1, exon 2, exon 3, exon 4, exon 5, exon 6, exon 7, exon 8, or SEQ ID NO: 79).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human SIRPα amino acid sequence (e.g., exon 1, exon 2, exon 3, exon 4, exon 5, exon 6, exon 7, exon 8, exon 9, or SEQ ID NO: 77).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human SIRPα amino acid sequence (e.g., exon 1, exon 2, exon 3, exon 4, exon 5, exon 6, exon 7, exon 8, exon 9, or SEQ ID NO: 77).

The present disclosure also provides a humanized SIRPα mouse amino acid sequence, wherein the amino acid sequence is selected from the group consisting of:

a) an amino acid sequence shown in SEQ ID NO: 53 or 62;

b) an amino acid sequence having a homology of at least 90% with or at least 90% identical to the amino acid sequence shown in SEQ ID NO: 53 or 62;

c) an amino acid sequence encoded by a nucleic acid sequence, wherein the nucleic acid sequence is able to hybridize to a nucleotide sequence encoding the amino acid shown in SEQ ID NO: 53 or 62 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 53 or 62;

e) an amino acid sequence that is different from the amino acid sequence shown in SEQ ID NO: 53 or 62 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 53 or 62.

The present disclosure also relates to a SIRPα nucleic acid (e.g., DNA or RNA) sequence, wherein the nucleic acid sequence can be selected from the group consisting of:

a) a nucleic acid sequence as shown in SEQ ID NO: 18, 50, 51, or 52, or a nucleic acid sequence encoding a homologous SIRPα amino acid sequence of a humanized mouse;

b) a nucleic acid sequence that is shown in SEQ ID NO: 18, 50, 51, or 52;

c) a nucleic acid sequence that is able to hybridize to the nucleotide sequence as shown in SEQ ID NO: 18, 50, 51, or 52 under a low stringency condition or a strict stringency condition;

d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 18, 50, 51, or 52;

e) a nucleic acid sequence that encodes an amino acid sequence, wherein the amino acid sequence has a homology of at least 90% with or at least 90% identical to the amino acid sequence shown in SEQ ID NO: 53 or 62;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 53 or 62;

g) a nucleic acid sequence that encodes an amino acid sequence, wherein the amino acid sequence is different from the amino acid sequence shown in SEQ ID NO: 53 or 62 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 53 or 62.

The present disclosure further relates to a SIRPα genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 18, 50, 51, or 52.

The disclosure also provides an amino acid sequence that has a homology of at least 90% with, or at least 90% identical to the sequence shown in SEQ ID NO: 53 or 62, and has protein activity. In some embodiments, the homology with the sequence shown in SEQ ID NO: 53 or 62 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 53 or 62 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 18, 50, 51, or 52, and encodes a polypeptide that has protein activity. In some embodiments, the homology with the sequence shown in SEQ ID NO: 18, 50, 51, or 52 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 18, 50, 51, or 52 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of identical residues (percent identity) and the percentage of residues conserved with similar physicochemical properties (percent homology), e.g. leucine and isoleucine, can be used to measure sequence similarity. Residues conserved with similar physicochemical properties are well known in the art. The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) SIRPα from an endogenous non-human SIRPα locus.

Additional details can be found in U.S. Patent Application Publication No. US 2019/0373867A1, which is incorporated herein by reference in its entirety.

### Humanized CD47 Animal

The present disclosure provides genetically-modified immune-deficient animal whose genome comprises at least one chromosome comprising a sequence encoding a human or chimeric CD47. CD47 is a ˜50 kDa heavily glycosylated, ubiquitously expressed membrane protein of the immunoglobulin superfamily with a single IgV-like domain at its N-terminus, a highly hydrophobic stretch with five membrane-spanning segments and an alternatively spliced cytoplasmic C-terminus. CD47, which was originally identified in association with αᵥβ₃ integrin, is also a member of the Ig superfamily of proteins, processing an IgV-like extracellular domain (N-terminal IgV domain), five putative membrane-spanning segments and short cytoplasmic tail.

Overexpression of CD47 has been found in nearly all types of tumors. Also, CD47 expression on cancer stem cells (CSCs) implies its role in cancer recurrence. It can increase the chance of CSC survival, which in turn could repopulate a new tumor mass and cause a tumor relapse.

CD47 down-regulation is also involved in the clearance of red blood cells (RBCs) and platelets by splenic macrophages, which may cause hemolytic anemia and idiopathic thrombocytopenic purpura, respectively. Thus, when CD47 antagonists are used as therapies, it is also very important to assess its toxicities.

A detailed description of CD47 and its function can be found, e.g., in Murata, Y. et al., “The CD47—SIRPα signalling system: its physiological roles and therapeutic application.” The Journal of Biochemistry 155.6 (2014): 335-344; Liu, Xiaojuan, et al. “Is CD47 an innate immune checkpoint for tumor evasion?.” Journal of hematology & oncology 10.1 (2017): 12; Huang et al. “Targeting CD47: the achievements and concerns of current studies on cancer immunotherapy.” Journal of thoracic disease 9.2 (2017): E168; which are incorporated by reference herein in the entirety.

In human genomes, CD47 gene (Gene ID: 961) locus has 11 exons, exon 1, exon 2, exon 3, exon 4, exon 5, exon 6, exon 7, exon 8, exon 9, exon 10, and exon 11. The CD47 protein has an extracellular N-terminal IgV domain, five transmembrane domains, a short C-terminal intracellular tail. In addition, it has two extracellular regions and two intracellular regions between neighboring transmembrane domains. The signal peptide is located at the extracellular N-terminal IgV domain of CD47. The nucleotide sequence for human CD47 mRNA is NM_001777.3 (SEQ ID NO: 80), and the amino acid sequence for human CD47 is NP_001768.1 (SEQ ID NO: 81). The location for each exon and each region in human CD47 nucleotide sequence and amino acid sequence is listed below:

TABLE 5

| | NM_001777.3 | NP_001768.1 |
|---|---|---|
| Human CD47 | 5346 bp | 323 aa |
| (approximate location) | (SEQ ID NO: 80) | (SEQ ID NO: 81) |
| Exon 1 | 1-226 | 1-15 |
| Exon 2 | 227-580 | 16-133 |
| Exon 3 | 581-670 | 134-163 |
| Exon 4 | 671-778 | 164-199 |
| Exon 5 | 779-871 | 200-230 |
| Exon 6 | 872-964 | 231-261 |
| Exon 7 | 965-1057 | 262-292 |
| Exon 8 | 1058-1089 | 293-303 |
| Exon9 | 1090-1114 | 304-311 |
| Exon10 | 1115-1147 | 312-322 |
| Exon11 | 1148-5346 | 323 |
| Signal peptide | 181-234 | 1-18 |
| Donor region in one | 247-558* | 23-126 |
| example | (with point mutation | |
| | 375(T→C)) | |

According to the information of human CD47 from the UniProt Database (UniProt identifier: Q08722), the extracellular Ig-like V-type domain corresponds to amino acids 19-127 of the human CD47 protein (SEQ ID NO: 81; or NP_001768.1), and the five transmembrane domains correspond to amino acids 142—Specifically, the extracellular Ig-like V-type domain is encoded by a portion of exon 2 of human CD47 gene. Thus, the donor region is located within the extracellular Ig-like V-type domain (N-terminal IgV domain).

In addition, according the information of human CD47 from the UniProt Database (UniProt identifier: Q08722), the extracellular region, the five membrane-spanning segments, and the C-terminal cytoplasmic tail correspond to amino acids 19-141, amino acids 142-289, and amino acids 290-323 of human CD47 protein (SEQ ID NO: 81; or NP_001768.1).

Human CD47 also have several transcript variants. These variants are summarized below.

TABLE 6

| Human CD47 transcript variants | Amino acid sequences |
|---|---|
| NM_001777.3 (5346bp) | NP_001768.1 (323 aa) |
| NM_198793.2 (5288bp) | NP_942088.1 (305 aa) |
| XM_005247909.1 (5021bp) | XP_005247966.1 (293 aa) |
| XM_005247908.1 (5078bp) | XP_005247965.1 (312 aa) |

In mice, CD47 gene locus has 10 exons, exon 1, exon 2, exon 3, exon 4, exon 5, exon 6, exon 7, exon 8, exon 9, and exon 10. The mouse CD47 protein also has an extracellular N-terminal IgV domain, five transmembrane domains, and a short C-terminal intracellular tail, and the signal peptide is located at the extracellular N-terminal IgV domain of CD47. The nucleotide sequence for mouse CD47 cDNA is NM_010581.3 (SEQ ID NO: 82), the amino acid sequence for mouse CD47 is NP_034711.1 (SEQ ID NO: 83). The location for each exon and each region in the mouse CD47 nucleotide sequence and amino acid sequence is listed below:

TABLE 7

| | NM_010581.3 | NP_034711.1 |
|---|---|---|
| Mouse CD47 | 1928 bp | 324 aa |
| (approximate location) | (SEQ ID NO: 82) | (SEQ ID NO: 83) |
| Exon 1 | 1-179 | 1-15 |
| Exon 2 | 180-527 | 16-131 |
| Exon 3 | 528-590 | 132-152 |
| Exon 4 | 591-680 | 153-182 |
| Exon 5 | 681-788 | 183-218 |
| Exon 6 | 789-881 | 219-249 |
| Exon 7 | 882-974 | 250-280 |
| Exon 8 | 975-1067 | 281-311 |
| Exon 9 | 1068-1099 | 312-322 |
| Exon 10 | 1100-1919 | 323-324 |
| Signal peptide | 134-187 | 1-18 |
| Replaced region in one example | 200-505 | 23-124 |

The mouse CD47 gene (Gene ID: 16423) is located in Chromosome 16 of the mouse genome, which is located from 49855253 to 49912424, of NC_000082.6 (GRCm38.p4 (GCF_000001635.24)). The 5′-UTR is from 49855618 to 49855786, exon 1 is from 49,855,618 to 49,855,832, the first intron is from 49,855,833 to 49,867,764, exon 2 is from 49,867,765 to 49,868,112, the second intron is from 49,868,113 to 49,869,017, exon 3 is from 49,869,018 to 49,869,080, the third intron is from 49,869,081 to 49,884,164, exon 4 is from 49,884,165 to 49,884,254, the fourth intron is from 49,884,255 to 49,894,176, exon 5 is from 49,894,177 to 49,894,284, the fifth intron is from 49,894,285 to 49,895,368, exon 6 is from 49,895,369 to 49,895,461, the sixth intron is from 49,895,462 to 49,896,355, exon 7 is from 49,896,356 to 49,896,448, the seventh intron is from 49,896,449 to 49,898,039, exon 8 is from 49,898,040 to 49,898,132, the eighth intron is from 49,898,133 to 49,906,780, exon 9 is from 49,906,781 to 49,906,812, the ninth intron is from 49,906,813 to 49,910,868, exon 10 is from 49,910,869 to 49,915,010, the 3′-UTR is from 49910878 to 49,915,010, based on transcript NM_010581.3. All relevant information for mouse CD47 locus can be found in the NCBI website with Gene ID: 16423, which is incorporated by reference herein in its entirety.

Like human CD47, the mouse CD47 has several transcript variants. A portion of these sequences can also be replaced by corresponding human sequences. Some exemplary sequences are shown in Table 8.

TABLE 8
Mouse CD47 sequence

| mRNA sequence | Amino acid sequence |
|---|---|
| NM_010581.3 (1928bp) | NP_034711.1 (324aa) |
| XM_006521809.3 (3101bp) | XP_006521872.1 (320aa) |
| XM_006521806.3 (3114bp) | XP_006521869.1 (342aa) |
| XM_006521807.3 (3081bp) | XP_006521870.1 (331aa) |
| XM_006521810.3 (3024bp) | XP_006521873.1 (312aa) |
| XM_006521808.3 (3051bp) | XP_006521871.1 (321aa) |
| XM_006521811.3 (2993bp) | XP_006521874.1 (303aa) |

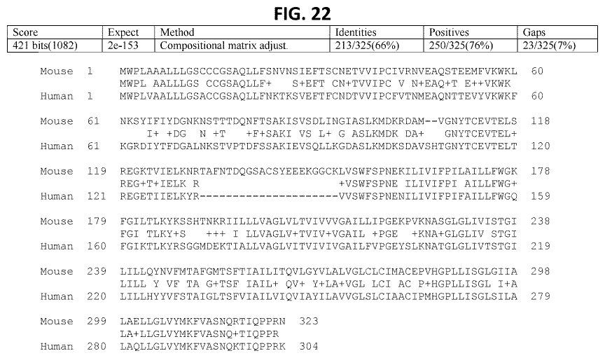

FIG. 22 shows the alignment between mouse CD47 amino acid sequence (NP_034711.1; SEQ ID NO: 83) and human CD47 amino acid sequence (NP_001768.1; SEQ ID NO: 81). Thus, the corresponding amino acid residue or region between human and mouse CD47 can also be found in FIG. 22.

CD47 genes, proteins, and locus of the other species are also known in the art. For example, the gene ID for CD47 in Rattus norvegicus is 29364, the gene ID for CD47 in Macaca mulatta (Rhesus monkey) is 704980, the gene ID for CD47 in Canis lupus familiaris (dog) is 478552, and the gene ID for CD47 in Cavia porcellus (domestic guinea pig) is 100727770. The relevant information for these genes (e.g., intron sequences, exon sequences, amino acid residues of these proteins) can be found, e.g., in NCBI database.

The present disclosure provides human or chimeric (e.g., humanized) CD47 nucleotide sequence and/or amino acid sequences. In some embodiments, the entire sequence of mouse exon 1, exon 2, exon 3, exon 4, exon 5, exon 6, exon 7, exon 8, exon 9, exon 10, signal peptide, the extracellular N-terminal IgV domain, the transmembrane domains (e.g., the first transmembrane domain, the second transmembrane domain, the third transmembrane domain, the fourth transmembrane domain, and/or the fifth transmembrane domain), and/or the C-terminal intracellular region are replaced by the corresponding human sequence. As used herein, the first transmembrane domain refers to the first transmembrane domain starting from the N-terminal of CD47. Similarly, the second, third, fourth, and fifth transmembrane domain refers to the second, third, fourth, and fifth transmembrane domain starting from the N-terminal of CD47.

In some embodiments, a “region” or “portion” of mouse exon 1, exon 2, exon 3, exon 4, exon 5, exon 6, exon 7, exon 8, exon 9, exon 10, signal peptide, the extracellular N-terminal IgV domain, the transmembrane domains (e.g., the first transmembrane domain, the second transmembrane domain, the third transmembrane domain, the fourth transmembrane domain, and/or the fifth transmembrane domain), and/or the C-terminal intracellular region is replaced by the corresponding human sequence.

In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, signal peptide, the extracellular N-terminal IgV domain, the transmembrane domains (e.g., the first transmembrane domain, the second transmembrane domain, the third transmembrane domain, the fourth transmembrane domain, and/or the fifth transmembrane domain), and/or the C-terminal intracellular region. In some embodiments, a region, a portion, or the entire sequence of mouse exon 2 is replaced by a region, a portion, or the entire sequence of human exon 2.

In some embodiments, a “region” or “portion” of mouse exon 1, exon 2, exon 3, exon 4, exon 5, exon 6, exon 7, exon 8, exon 9, exon 10, signal peptide, the extracellular N-terminal IgV domain, the transmembrane domains (e.g., the first transmembrane domain, the second transmembrane domain, the third transmembrane domain, the fourth transmembrane domain, and/or the fifth transmembrane domain), and/or the C-terminal intracellular region is deleted.

Thus, in some embodiments, the present disclosure also provides a chimeric (e.g., humanized) CD47 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD47 mRNA sequence (e.g., SEQ ID NO: 82), mouse CD47 amino acid sequence (e.g., SEQ ID NO: 83), or a portion thereof (e.g., exon 1, exon 2, exon 3, exon 4, exon 5, exon 6, exon 7, exon 8, exon 9, or exon 10); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD47 mRNA sequence (e.g., SEQ ID NO: 80), human CD47 amino acid sequence (e.g., SEQ ID NO: 81), or a portion thereof (e.g., exon 1, exon 2, exon 3, exon 4, exon 5, exon 6, exon 7, exon 8, exon 9, exon 10, or exon 11).

In some embodiments, the sequence encoding amino acids 23-124 of mouse CD47 (SEQ ID NO: 83) is replaced. In some embodiments, the sequence is replaced by a sequence encoding a corresponding region of human CD47 (e.g., amino acids 23-126 of human CD47 (SEQ ID NO: 81)).

In some embodiments, a sequence encoding the entire or part of the extracellular N-terminal IgV domain of mouse CD47 (SEQ ID NO: 83) is replaced. In some embodiments, the sequence is replaced by a sequence encoding a corresponding region (e.g., the entire or part of the extracellular N-terminal IgV domain) of human CD47 (SEQ ID NO: 81). In some embodiments, the animal has a humanized IgV-like extracellular domain (N-terminal IgV domain).

In some embodiments, the nucleic acids as described herein are operably linked to a promotor or regulatory element, e.g., an endogenous mouse CD47 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD47 nucleotide sequence (e.g., exon 1, exon 2, exon 3, exon 4, exon 5, exon 6, exon 7, exon 8, exon 9, exon 10, or SEQ ID NO: 82).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD47 nucleotide sequence (e.g., exon 1, exon 2, exon 3, exon 4, exon 5, exon 6, exon 7, exon 8, exon 9, exon 10, or SEQ ID NO: 82).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD47 nucleotide sequence (e.g., exon 1, exon 2, exon 3, exon 4, exon 5, exon 6, exon 7, exon 8, exon 9, exon 10, exon 11, or SEQ ID NO: 80).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD47 nucleotide sequence (e.g., exon 1, exon 2, exon 3, exon 4, exon 5, exon 6, exon 7, exon 8, exon 9, exon 10, exon 11, or SEQ ID NO: 80).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD47 amino acid sequence (e.g., exon 1, exon 2, exon 3, exon 4, exon 5, exon 6, exon 7, exon 8, exon 9, exon 10, or SEQ ID NO: 83).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD47 amino acid sequence (e.g., exon 1, exon 2, exon 3, exon 4, exon 5, exon 6, exon 7, exon 8, exon 9, exon 10, or SEQ ID NO: 83).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD47 amino acid sequence (e.g., exon 1, exon 2, exon 3, exon 4, exon 5, exon 6, exon 7, exon 8, exon 9, exon 10, exon 11, or SEQ ID NO: 81).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD47 amino acid sequence (e.g., exon 1, exon 2, exon 3, exon 4, exon 5, exon 6, exon 7, exon 8, exon 9, exon 10, exon 11, or SEQ ID NO: 81).

In some embodiments, the percentage identity with the sequence shown in SEQ ID NO: 75 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

Cells, tissues, and animals (e.g., mouse) are also provided that comprise the nucleotide sequences as described herein, as well as cells, tissues, and animals (e.g., mouse) that express human or chimeric (e.g., humanized) CD47 from an endogenous non-human CD47 locus.

In one aspect, the disclosure provides a genetically-modified, non-human animal whose genome comprises at least one chromosome comprising a sequence encoding a human or chimeric CD47.

In some embodiments, the sequence encoding the human or chimeric CD47 is operably linked to an endogenous regulatory element at the endogenous CD47 gene locus in the at least one chromosome.

In some embodiments, the sequence encoding a human or chimeric CD47 comprises a sequence encoding an amino acid sequence that is at least 50%, 55%, 65%, 70%, 75%, 80%, 85%, 90%, 95%, 99%, or 100% identical to human CD47 (SEQ ID NO: 81).

In some embodiments, the sequence encoding a human or chimeric CD47 comprises a sequence encoding an amino acid sequence that is at least 50%, 55%, 65%, 70%, 75%, 80%, 85%, 90%, 95%, 99%, or 100% identical to SEQ ID NO: 75.

In some embodiments, the sequence encoding a human or chimeric CD47 comprises a sequence that is at least 50%, 55%, 65%, 70%, 75%, 80%, 85%, 90%, 95%, 99%, or 100% identical to amino acids 23-126 of SEQ ID NO: 81.

In some embodiments, the animal is a mammal, e.g., a monkey, a rodent or a mouse. In some embodiments, the animal is a BALB/c mouse or a C57BL/6 mouse.

In some embodiments, the animal does not express endogenous CD47. In some embodiments, the animal has one or more cells expressing human or chimeric CD47.

In some embodiments, the animal has one or more cells expressing human or chimeric CD47, and the expressed human or chimeric CD47 can bind to endogenous SIRPα. In some embodiments, the animal has one or more cells expressing human or chimeric CD47, and the expressed human or chimeric CD47 cannot bind to endogenous SIRPα.

In another aspect, the disclosure is related to a genetically-modified, non-human animal, wherein the genome of the animal comprises a replacement of a sequence encoding a region of endogenous CD47 with a sequence encoding a corresponding region of human CD47 at an endogenous CD47 gene locus.

In some embodiments, the sequence encoding the corresponding region of human CD47 is operably linked to an endogenous regulatory element at the endogenous CD47 locus, and one or more cells of the animal expresses a chimeric CD47.

In some embodiments, the animal does not express endogenous CD47. In some embodiments, the replaced locus is the extracellular N-terminal IgV domain of CD47.

In some embodiments, the animal has one or more cells expressing a chimeric CD47 having an extracellular N-terminal IgV domain, wherein the extracellular N-terminal IgV domain comprises a sequence that is at least 50%, 60%, 70%, 80%, 90%, 95%, or 99% identical to the extracellular N-terminal IgV domain of human CD47.

In some embodiments, the extracellular N-terminal IgV domain of the chimeric CD47 has a sequence that has at least 10, 20, 30, 40, 50, 60, 70, 80, 90, or 100 contiguous amino acids that are identical to a contiguous sequence present in the extracellular N-terminal IgV domain of human CD47.

In some embodiments, the animal is a mouse, and the replaced endogenous CD47 locus is exon 1, exon 2, exon 3, exon 4, exon 5, exon 6, exon 7, exon 8, exon 9, and/or exon 10 of the endogenous mouse CD47 gene.

In some embodiments, the animal is heterozygous with respect to the replacement at the endogenous CD47 gene locus. In some embodiments, the animal is homozygous with respect to the replacement at the endogenous CD47 gene locus.

In another aspect, the disclosure is related to methods for making a genetically-modified, non-human animal. The methods involve replacing in at least one cell of the animal, at an endogenous CD47 gene locus, a sequence encoding a region of an endogenous CD47 with a sequence encoding a corresponding region of human CD47.

In some embodiments, the sequence encoding the corresponding region of human CD47 comprises exon 2 of a human CD47 gene.

In some embodiments, the sequence encoding the corresponding region of CD47 comprises at least 100, 150, 200, 250, or 300 nucleotides of exon 2 of a human CD47 gene.

In some embodiments, the sequence encoding the corresponding region of human CD47 encodes a sequence that is at least 90% identical to amino acids 23-126 of SEQ ID NO: 81.

In some embodiments, the locus is located within the extracellular N-terminal IgV domain of CD47.

In some embodiments, the animal is a mouse, and the locus is exon 1, exon 2, exon 3, exon 4, exon 5, exon 6, exon 7, exon 8, exon 9, and/or exon 10 of the mouse CD47 gene (e.g., exon 2).

In another aspect, the disclosure is also related to a non-human animal comprising at least one cell comprising a nucleotide sequence encoding a chimeric CD47 polypeptide, wherein the chimeric CD47 polypeptide comprises at least 50 contiguous amino acid residues that are identical to the corresponding contiguous amino acid sequence of a human CD47, wherein the animal expresses the chimeric CD47.

In some embodiments, the chimeric CD47 polypeptide has at least 50 contiguous amino acid residues that are identical to the corresponding contiguous amino acid sequence of a human CD47 extracellular N-terminal IgV domain.

In some embodiments, the chimeric CD47 polypeptide comprises a sequence that is at least 90%, 95%, or 99% identical to amino acids 23-126 of SEQ ID NO: 81.

In some embodiments, the nucleotide sequence is operably linked to an endogenous CD47 regulatory element of the animal.

In some embodiments, the chimeric CD47 polypeptide comprises five endogenous CD47 transmembrane regions and/or an endogenous CD47 C-terminal intracellular tail.

In some embodiments, the nucleotide sequence is integrated to an endogenous CD47 gene locus of the animal.

In some embodiments, the chimeric CD47 has at least one mouse CD47 activity and/or at least one human CD47 activity.

In another aspect, the disclosure is also related to methods of making a genetically-modified mouse cell that expresses a chimeric CD47. The methods involve replacing, at an endogenous mouse CD47 gene locus, a nucleotide sequence encoding a region of mouse CD47 with a nucleotide sequence encoding a corresponding region of human CD47, thereby generating a genetically-modified mouse cell that includes a nucleotide sequence that encodes the chimeric CD47, wherein the mouse cell expresses the chimeric CD47.

In some embodiments, the chimeric CD47 comprises: an extracellular N-terminal IgV domain of human CD47; and one or more transmembrane domains of mouse CD47 and/or a C-terminal intracellular tail of mouse CD47.

In some embodiments, the nucleotide sequence encoding the chimeric CD47 is operably linked to an endogenous CD47 regulatory region, e.g., promoter.

In some embodiments, the animal further comprises a sequence encoding an additional human or chimeric protein (e.g., SIRPα, programmed cell death protein 1 (PD-1), cytotoxic lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, or TNF Receptor Superfamily Member 4 (OX40)).

In some embodiments, the additional human or chimeric protein is SIRPα and/or PD-1.

In one aspect, the disclosure also provides methods of determining effectiveness of a CD47 antagonist (e.g., an anti-CD47 antibody) for the treatment of cancer. The methods involve administering the CD47 antagonist to the animal described herein, wherein the animal has a tumor; and determining the inhibitory effects of the CD47 antagonist to the tumor. In some embodiments, the animal comprises one or more cells that express SIRPα. In some embodiments, the tumor comprises one or more cells that express CD47.

In some embodiments, the tumor comprises one or more cancer cells that are injected into the animal.

In some embodiments, determining the inhibitory effects of the CD47 antagonist (e.g., an anti-CD47 antibody) to the tumor involves measuring the tumor volume in the animal.

In some embodiments, the tumor cells are melanoma cells, non-small cell lung carcinoma (NSCLC) cells, small cell lung cancer (SCLC) cells, non-Hodgkin lymphoma cells, bladder cancer cells, prostate cancer cells, breast cancer cells, ovarian cancer cells, colorectal cancer cells, and/or refractory solid tumor cells.

In another aspect, the disclosure also provides methods of determining effectiveness of a CD47 antagonist (e.g., an anti-CD47 antibody) and an additional therapeutic agent for the treatment of a tumor. The methods involve administering the CD47 antagonist and the additional therapeutic agent to the animal as described herein, wherein the animal has a tumor; and determining the inhibitory effects on the tumor.

In some embodiments, the animal further comprises a sequence encoding a human or chimeric SIRPα.

In some embodiments, the additional therapeutic agent is an anti-SIRPα antibody. In some embodiments the additional therapeutic agent is an anti-PD-1 antibody, an anti-PD-L1 antibody, an anti-CTLA4 antibody, an anti-CD20 antibody, an anti-EGFR antibody, or an anti-CD319 antibody.

In some embodiments, the tumor comprises one or more tumor cells that express CD47.

In some embodiments, the tumor is caused by injection of one or more cancer cells into the animal.

In some embodiments, determining the inhibitory effects of the treatment involves measuring the tumor volume in the animal.

In some embodiments the tumor comprises melanoma cells, non-small cell lung carcinoma (NSCLC) cells, small cell lung cancer (SCLC) cells, non-Hodgkin lymphoma cells, bladder cancer cells, prostate cancer cells, breast cancer cells, ovarian cancer cells, colorectal cancer cells, and/or refractory solid tumor cells.

In another aspect, the disclosure further provides methods of determining toxicity of an agent (e.g., a CD47 antagonist). The methods involve administering the agent to the animal as described herein; and determining weight change of the animal. In some embodiments, the method further involve performing a blood test (e.g., determining red blood cell count).

In one aspect, the disclosure relates to proteins comprising an amino acid sequence, wherein the amino acid sequence is one of the following:
(a) an amino acid sequence set forth in SEQ ID NO: 75;
(b) an amino acid sequence that is at least 90%, 91%, 92%, 93%, 94%, 95%, 96%, 97%, 98%, or 99% identical to SEQ ID NO: 75;
(c) an amino acid sequence that is different from the amino acid sequence set forth in SEQ ID NO: 75 by no more than 10, 9, 8, 7, 6, 5, 4, 3, 2 or 1 amino acid; and
(d) an amino acid sequence that comprises a substitution, a deletion and/or insertion of one, two, three, four, five or more amino acids to the amino acid sequence set forth in SEQ ID NO: 75.

In some embodiments, provided herein are cells comprising the proteins disclosed herein. In some embodiments, provided herein are animals having the proteins disclosed herein. In another aspect, the disclosure relates to nucleic acids comprising a nucleotide sequence, wherein the nucleotide sequence is one of the following:
(a) a sequence that encodes the protein as described herein;
(b) SEQ ID NO: 72, SEQ ID NO: 73 or SEQ ID NO: 74;
(c) a sequence that is at least 90%, 91%, 92%, 93%, 94%, 95%, 96%, 97%, 98%, or 99% identical to SEQ ID NO: 72, SEQ ID NO: 73 or SEQ ID NO: 74.

In some embodiments, provided herein are cells comprising the nucleic acids disclosed herein. In some embodiments, provided herein are animals having the nucleic acids disclosed herein.

In another aspect, the disclosure also provides a genetically-modified, non-human animal whose genome comprise a disruption in the animal's endogenous CD47 gene, wherein the disruption of the endogenous CD47 gene comprises deletion of exon 2 or part thereof of the endogenous CD47 gene.

In some embodiments, the disruption of the endogenous CD47 gene further comprises deletion of one or more exons or part of exons selected from the group consisting of exon 1, exon 3, exon 4, exon 5, exon 6, exon 7, exon 8, exon 9, and exon 10 of the endogenous CD47 gene.

In some embodiments, the disruption of the endogenous CD47 gene further comprises deletion of one or more introns or part of introns selected from the group consisting of intron 1, intron 2, intron 3, intron 4, intron 5, intron 6, intron 7, intron 8, and intron 9 of the endogenous CD47 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous CD47 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, exon 8, exon 9, or exon 10 (e.g., deletion of at least 300 nucleotides of exon 2).

Additional details (e.g., genetically-modified animals, vectors, methods of making the genetically-modified animals, and methods of using the genetically-modified animals) can be found in U.S. Patent Application Publication No. US 2019/0343097A1, which is incorporated herein by reference in its entirety.

### CD132 (Interleukin-2 Receptor Subunit Gamma or IL2RG)

The present disclosure provides various immune-deficient animals. In some embodiments, the immune-deficient animal has a disruption at CD132 gene. CD132, also known as interleukin-2 receptor subunit gamma or IL2RG, is a cytokine receptor sub-unit that is common to the receptor complexes for IL-2, IL-4, IL-7, IL-9, IL-15 and IL-21. These receptors are members of the type I cytokine receptor family expressed on most lymphocyte populations. Interleukin-2 (IL-2) is a 15.5 kDa type 1 four α-helical bundle cytokine produced primarily by CD4+ T cells following their activation by antigen. IL-2 was the first type 1 cytokine cloned and the first cytokine for which a receptor component was cloned. Three different IL-2 receptor chains exist that together generate low, intermediate, and high affinity IL-2 receptors. The ligand-specific IL-2 receptor α chain (IL-2Rα, CD25, Tac antigen), which is expressed on activated but not non-activated lymphocytes, binds IL-2 with low affinity (Kd ˜10⁻⁸ M); the combination of IL-2R13 (CD122) and IL-2Rγ (CD132) together form an IL-2Rβ/γc complex mainly on memory T cells and NK cells that binds IL-2 with intermediate affinity (Kd ˜10⁻⁹ M); and when all three receptor chains are co-expressed on activated T cells and Treg cells, IL-2 is bound with high affinity (Kd ˜10⁻¹¹M).

For the high affinity receptor, the three dimensional structure of the quaternary complex supports a model wherein IL-2 initially bind IL-2Ra, then IL-2R13 is recruited, and finally IL-2Rγ. The intermediate and high affinity receptor forms are functional, transducing IL-2 signals.

CD132 is also an essential component shared by the receptors for IL-2, IL-4, IL-7, IL-9, IL-15, and IL-21.

IL-2Rγ is encoded by the gene, IL2RG (CD132), that is mutated in humans with X-linked severe combined immunodeficiency (XSCID) and physically recruits JAK3, which when mutated also causes an XSCID-like T−B+NK− form of SCID. In XSCID and JAK3-deficient SCID, the lack of signaling by IL-7 and IL-15, respectively, explains the lack of T and NK cell development, whereas defective signaling by IL-4 and IL-21 together explain the non-functional B cells and hypogammaglobulinemia.

A detailed description of CD132 and its function can be found, e.g., in Liao et al. “IL-2 family cytokines: new insights into the complex roles of IL-2 as a broad regulator of T helper cell differentiation,” Current opinion in immunology 23.5 (2011): 598-604; Noguchi et al. “Interleukin-2 receptor gamma chain: a functional component of the interleukin-7 receptor,” Science 262.5141 (1993): 1877-1880; Henthorn et al. “IL-2Rγ gene microdeletion demonstrates that canine X-linked severe combined immunodeficiency is a homologue of the human disease,” Genomics 23.1 (1994): 69-74; and U.S. Pat. No. 7,145,055; each of which is incorporated herein by reference in its entirety.

In human genomes, CD132 gene (Gene ID: 3561) is located on X chromosome, and has eight exons, exon 1, exon 2, exon 3, exon 4, exon 5, exon 6, exon 7, and exon 8. The CD132 protein also has an extracellular region, a transmembrane region, and a cytoplasmic region. The nucleotide sequence for human CD132 mRNA is NM_000206.2, and the amino acid sequence for human CD132 is NP_000197.1.

Similarly, in mice, CD132 gene locus has eight exons, exon 1, exon 2, exon 3, exon 4, exon 5, exon 6, exon 7, and exon 8. The CD132 protein also has an extracellular region, a transmembrane region, and a cytoplasmic region, and the signal peptide is located at the extracellular region of CD132. The nucleotide sequence for mouse CD132 mRNA is NM_013563.4 (SEQ ID NO: 84), the amino acid sequence for mouse CD132 is NP_038591.1 (SEQ ID NO: 71). The location for each exon and each region in the mouse CD132 nucleotide sequence and amino acid sequence is listed below:

TABLE 9

| | NM_013563.4 | NP_038591.1 |
|---|---|---|
| Mouse CD132 | 1663bp | 369aa |
| (approximate location) | (SEQ ID NO: 84) | (SEQ ID NO: 71) |
| Exon 1 | 1-201 | 1-38 |
| Exon 2 | 202-355 | 39-90 |
| Exon 3 | 356-540 | 91-151 |
| Exon 4 | 541-683 | 152-199 |
| Exon 5 | 684-846 | 200-253 |
| Exon 6 | 847-943 | 254-286 |
| Exon 7 | 944-1010 | 287-308 |
| Exon 8 | 1011-1663 | 309-369 |
| Signal peptide | 87-158 | 1-24 |
| Extracellular region | 159-875 | 25-263 |
| (excluding signal peptide region) | | |
| Transmembrane region | 876-938 | 264-284 |
| Cytoplasmic region | 939-1193 | 285-369 |

The mouse CD132 gene (Gene ID: 16186) is located in Chromosome X of the mouse genome, which is located from 101,268,255 to 101,264,385 of NC_000086.7 (GRCm38.p4 (GCF_000001635.24)). The 5′-UTR is from 101,268,255 to 101,268,170, exon 1 is from 101,268,255 to 101,268,055, the first intron (intron 1) is from 101,268,054 to 101,267,865, exon 2 is from 101,267,864 to 101,267,711, the second intron (intron 2) is from 101,267,710 to 101,267,496, exon 3 is from 101,267,495 to 101,267,311, the third intron (intron 3) is from 101,267,310 to 101,267,121, exon 4 is from 101,267,120 to 101,266,978, the fourth intron (intron 4) is from 101,266,977 to 101,266,344, exon 5 is from 101,266,343 to 101,266,181, the fifth intron (intron 5) is from 101,266,180 to 101,265,727, exon 6 is from 101,265,726 to 101,265,630, the sixth intron (intron 6) is from 101,265,629 to 101,265,443, exon 7 is from 101,265,442 to 101,265,376, the seventh intron (intron 7) is from 101,265,375 to 101,265,038, exon 8 is from 101,265,037 to 101,264,378, and the 3′-UTR is from 101,264,851 to 101,264,378, based on transcript NM_013563.4. All relevant information for mouse CD132 locus can be found in the NCBI website with Gene ID: 16186, which is incorporated by reference herein in its entirety.

CD132 genes, proteins, and locus of the other species are also known in the art. For example, the gene ID for CD132 in Rattus norvegicus is 140924, the gene ID for CD132 in Macaca mulatta (Rhesus monkey) is 641338, the gene ID for CD132 in Sus scrofa (pig) is 397156. The relevant information for these genes (e.g., intron sequences, exon sequences, amino acid residues of these proteins) can be found, e.g., in NCBI database.

The present disclosure provides a genetically-modified, non-human animal whose genome comprise a disruption in the animal's endogenous CD132 gene, wherein the disruption of the endogenous CD132 gene comprises deletion of one or more exons, or part of the one or more exons, wherein the one or more exons are selected from the group consisting of exon 1, exon 2, exon 3, exon 4, exon 5, exon 6, exon 7, and exon 8 of the endogenous CD132 gene. Thus, the disclosure provides a genetically-modified, non-human animal that does not have one or more exons that are selected from the group consisting of exon 1, exon 2, exon 3, exon 4, exon 5, exon 6, exon 7, and exon 8 of the endogenous CD132 gene.

As used herein, the term “deletion of an exon” refers to the deletion the entire exon. For example, deletion of exon 2 means that all sequences in exon 2 are deleted.

As used herein, the term “deletion of part of an exon” refers to at least one nucleotide, but not all nucleotides in the exon is deleted. In some embodiment, at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleotides in the exon are deleted.

In some embodiments, the disruption comprises deletion of one or more introns, or part of the one or more introns, wherein the one or more introns are selected from the group consisting of intron 1, intron 2, intron 3, intron 4, intron 5, intron 6, and intron 7 of the endogenous CD132 gene. Thus, the disclosure provides a genetically-modified, non-human animal does not have one or more introns that are selected from the group consisting of intron 1, intron 2, intron 3, intron 4, intron 5, intron 6, and intron 7 of the endogenous CD132 gene.

In some embodiments, the disruption of the endogenous CD132 gene comprises deletion of exon 2 of the endogenous CD132 gene. In some embodiments, the disruption of the endogenous CD132 gene further comprises deletion of exon 1, or part of exon 1 of the endogenous CD132 gene.

In some embodiments, the entire sequence of mouse exon 1, exon 2, exon 3, exon 4, exon 5, exon 6, exon 7, and exon 8 are deleted. In some embodiments, the signal peptide region, extracellular region, transmembrane region, and/or cytoplasmic region of CD132 are deleted.

In some embodiments, a “region” or “portion” of mouse exon 1, exon 2, exon 3, exon 4, exon 5, exon 6, exon 7, exon 8, intron 1, intron 2, intron 3, intron 4, intron 5, intron 6, and intron 7, signal peptide region, extracellular region, transmembrane region, and/or cytoplasmic region are deleted. The term “region” or “portion” can refer to at least 1, 2, 3, 4, 5, 6, 7, 8, 9, 10, 20, 30, 40, 50, 60, 70, 80, 90, 100, 110, 120, 130, 150, 200, 250, 300, 350, or 400 nucleotides.

In some embodiments, the “region” or “portion” can be at least 10%, 20%, 30%, 40%, 50%, 55%, 60%, 65%, 70%, 75%, 80%, 85%, 90%, 95%, or 99% of exon 1, exon 2, exon 3, exon 4, exon 5, exon 6, exon 7, exon 8, intron 1, intron 2, intron 3, intron 4, intron 5, intron 6, and intron 7, signal peptide region, extracellular region, transmembrane region, or cytoplasmic region. In some embodiments, a region, a portion, or the entire sequence of mouse exon 1, exon 2, exon 3, exon 4, exon 5, exon 6, exon 7, and/or exon 8 are deleted. In some embodiments, a region, a portion, or the entire sequence of mouse intron 1, intron 2, intron 3, intron 4, intron 5, intron 6, and/or intron 7 are deleted.

In some embodiments, the disruption comprises or consists of deletion of more than 100, 110, 120, 130, 140, 150, 160, 170, 180, 190, 200, 250, 300, 350, 400, 450, 500, 550, 600, or 650 nucleotides in exon 1, exon 2, exon 3, exon 4, exon 5, exon 6, exon 7, and/or exon 8. In some embodiments, the disruption comprises or consists of deletion of more than 100, 110, 120, 130, 140, 150, 160, 170, 180, 190, 200, 250, 300, 350, 400, 450, 500, 550, 600, 650, 700, 800, 900, or 1000 nucleotides in intron 1, intron 2, intron 3, intron 4, intron 5, intron 6, and/or intron 7.

In some embodiments, the disruption comprises or consists of deletion of more than 100, 110, 120, 130, 140, 150, 160, 170, 180, 190, or 200 nucleotides (e.g., about 150 or 160 nucleotides) in exon 1; deletion of the entirety of intron 1, exon 2, intron 2, exon 3, intron 3, exon 4, intron 4, exon 5, intron 5, exon 6, intron 6, exon 7, intron 7; and/or deletion of more than 100, 110, 120, 130, 140, 150, 160, 170, 180, 190, 200, 210, 220, 230, 240, 250, 260, 270, 280, 290, 300, 350, 400, 450, 500, 550, 600, or 650 nucleotides (e.g., about 200, 250 or 270 nucleotides) in exon 8.

In some embodiments, the length of the remaining exon sequences at the endogenous CD132 gene locus is less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50% of the total length of all exon sequences of the endogenous CD132 gene. In some embodiments, the length of the remaining exon sequences at the endogenous CD132 gene locus is more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50% of the total length of all exon sequences of the endogenous CD132 gene.

In some embodiments, the length of the remaining sequences at that the endogenous CD132 gene locus is less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50% of the full sequence of the endogenous CD132 gene. In some embodiments, the length of the remaining sequences at that the endogenous CD132 gene locus is more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50% of the full sequence of the endogenous CD132 gene.

The present disclosure further relates to the genomic DNA sequence of a CD132 knockout mouse. In some embodiments, the genome of the animal comprises from 5′ to 3′ at the endogenous CD132 gene locus, (a) a first DNA sequence; optionally; (b) a second DNA sequence comprising an exogenous sequence; (c) a third DNA sequence, wherein the first DNA sequence, the optional second DNA sequence, and the third DNA sequence are linked.

The second DNA sequence can have a length of 0 nucleotides to 300 nucleotides (e.g., about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290 or 300 nucleotides). In some embodiments, the second DNA sequence has only 0 nucleotides, which means that there is no extra sequence between the first DNA sequence and the third DNA sequence. In some embodiments, the second DNA sequence has at least 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290 or 300 nucleotides. In some embodiments, the second DNA sequence has at most 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290 or 300 nucleotides.

In some embodiments, the first DNA sequence comprises an endogenous CD132 gene sequence that is located upstream of intron 1, and can include all or just part of sequences that is located upstream of intron 1. In some embodiments, the first DNA sequence comprises an endogenous CD132 gene sequence that is located upstream of exon 1. In some embodiments, the first DNA sequence comprises a sequence that has a length (5′ to 3′) of from 10 to 200 nucleotides (e.g., from 10 to 100 nucleotides, or from 10 to 20 nucleotides) starting from the first nucleotide in exon 1 of the CD132 gene to the last nucleotide of the first DNA sequence. In some embodiments, the first DNA sequence comprises at least 5, 10, 11, 12, 13, 14, 15, 16, 17, 18, 19, 20, 30, 40, or 50, 60, 70, 80, 90, 100, 110, 120, 130, 140, 150, 160, 170, 180, 190, or 200 nucleotides from exon 1. In some embodiments, the first DNA sequence has at most 5, 10, 11, 12, 13, 14, 15, 16, 17, 18, 19, 20, 30, 40, or 50, 60, 70, 80, 90, 100, 110, 120, 130, 140, 150, 160, 170, 180, 190, or 200 nucleotides from exon 1.

In some embodiments, the third DNA sequence comprises an endogenous CD132 gene sequence that is located downstream of the last intron (e.g., intron 7 in mouse), and can include all or just part of sequences that is located downstream of intron 7. In some embodiments, the third DNA sequence comprises a sequence that has a length (5′ to 3′) of from 200 to 600 nucleotides (e.g., from 300 to 400 nucleotides, or from 350 to 400 nucleotides) starting from the first nucleotide in the third DNA sequence to the last nucleotide in the last exon (e.g., exon 8 in mouse) of the endogenous CD132 gene. In some embodiments, the third DNA sequence comprises at least 100, 150, 200, 250, 300, 350, 400, 450, 500, 550, 600, or 650 nucleotides from the last exon (e.g., exon 8 in mouse). In some embodiments, the third DNA sequence has at most 100, 150, 200, 250, 300, 350, 400, 450, 500, 550, 600, or 650 nucleotides from the last exon (e.g., exon 8 in mouse).

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein (e.g., exon sequences, intron sequences, the remaining exon sequences, the deleted sequences), and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein (e.g., amino acid sequences encoded by exons). In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, or 500 nucleotides. In some embodiments, the amino acid sequence is less than 5, 6, 7, 8, 9, 10, 20, 30, 40, 50, 60, 70, 80, 90, 100, 110, 120, 130, or 150 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

Cells, tissues, and animals (e.g., mouse) are also provided that comprise a disruption of the endogenous CD132 gene as described herein, as well as cells, tissues, and animals (e.g., mouse) that have any nucleic acid sequence as described herein.

Additional details (e.g., the genetically-modified animals, vectors, methods of making the genetically-modified animals, methods of using the genetically-modified animals, and CD132 knockout model with additional genetic modifications) can be found, e.g., in U.S. Pat. No. 10,820,580B2, which is incorporated herein by reference in its entirety.

### Genetically Modified Animals

As used herein, the term “genetically-modified non-human animal” refers to a non-human animal having genetic modification (e.g., exogenous DNA) in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the genetic modification in its genome. The cell having exogenous DNA can be various kinds of cells, e.g., an endogenous cell, a somatic cell, an immune cell, a T cell, a B cell, a germ cell, a blastocyst, or an endogenous tumor cell. In some embodiments, genetically-modified non-human animals are provided that comprise a modified endogenous SIRPα locus that comprises an exogenous sequence (e.g., a human sequence), e.g., a replacement of one or more non-human sequences with one or more human sequences. In some embodiments, genetically-modified non-human animals are provided that comprise a disruption or a deletion at the endogenous CD132 locus. The animals are generally able to pass the modification to progeny, i.e., through germline transmission. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one portion of the sequences of the protein or the polypeptide does not correspond to wildtype amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized SIRPα gene or a humanized SIRPα nucleic acid. In some embodiments, at least one or more portions of the gene or the nucleic acid is from the human SIRPα gene, at least one or more portions of the gene or the nucleic acid is from a non-human SIRPα gene. In some embodiments, the gene or the nucleic acid comprises a sequence that encodes a SIRPα protein. The encoded SIRPα protein is functional or has at least one activity of the human SIRPα protein or the non-human SIRPα protein, e.g., binding to human or non-human CD47, phosphorylation of its cytoplasmic ITIM motif after binding to CD47, inhibiting phagocytosis, and/or downregulating immune response.

In some embodiments, the chimeric protein or the chimeric polypeptide is a humanized SIRPα protein or a humanized SIRPα polypeptide. In some embodiments, at least one or more portions of the amino acid sequence of the protein or the polypeptide is from a human SIRPα protein, and at least one or more portions of the amino acid sequence of the protein or the polypeptide is from a non-human SIRPα protein. The humanized SIRPα protein or the humanized SIRPα polypeptide is functional or has at least one activity of the human SIRPα protein or the non-human SIRPα protein.

In some embodiments, the humanized SIRPα protein or the humanized SIRPα polypeptide can bind to mouse CD47, inhibit phagocytosis, and/or downregulate immune response. In some embodiments, the humanized SIRPα protein or the humanized SIRPα polypeptide cannot bind to mouse CD47, thus cannot inhibit phagocytosis.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2006, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/01a. In some embodiments, the mouse is a 129 strain selected from the group consisting of a strain that is 129P1, 129P2, 129P3, 129X1, 129S1 (e.g., 12951/SV, 12951/SvIm), 129S2, 129S4, 12955, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the genetically-modified animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes).

Non-limiting examples of such mice include, e.g., B-NDG mice, NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human SIRPα locus, and further comprises a modification that compromises, inactivates, or destroys the immune system (or one or more cell types of the immune system) of the non-human animal in whole or in part. In some embodiments, modification is, e.g., selected from the group consisting of a modification that results in B-NDG mice, NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature SIRPα coding sequence with human mature SIRPα coding sequence.

The mouse genetic background can also affect the interaction of CD47 and SIRPα in the mouse. In mice with C57BL/6 background, the mouse SIRPα has a relatively weak binding affinity with humanized or human CD47 protein. In contrast, in mice with BALB/c background, the binding affinity between mouse SIRPα and human (or humanized) CD47 protein is similar to the binding affinity between mouse SIRPα and mouse CD47 protein. Thus, in some embodiments, the humanized CD47 mouse with C57BL/6 background can be used to test the toxicity of anti-hCD47 antibodies. In some embodiments, the humanized CD47 mouse with BALB/c background can be used to test the toxicity of anti-hCD47 antibodies and/or the efficacy of anti-hCD47 antibodies in terms of inhibiting tumor growth. In some embodiments, mice (any background) with both humanized CD47 and humanized SIRPα can be used to test the toxicity of anti-hCD47 antibodies and/or the efficacy of anti-hCD47 antibodies in terms of inhibiting tumor growth.

Genetically modified non-human animals can comprise a modification of an endogenous non-human SIRPα locus. In some embodiments, the modification can comprise a human nucleic acid sequence encoding at least a portion of a mature SIRPα protein (e.g., at least 10%, 20%, 30%, 40%, 50%, 60%, 70%, 80%, 90%, 95%, 96%, 97%, 98%, or 99% identical to the mature SIRPα protein sequence). Although genetically modified cells are also provided that can comprise the modifications described herein (e.g., ES cells, somatic cells), in many embodiments, the genetically modified non-human animals comprise the modification of the endogenous SIRPα locus in the germline of the animal.

Genetically modified animals can express a human SIRPα and/or a chimeric (e.g., humanized) SIRPα from endogenous mouse loci, wherein the endogenous mouse SIRPα gene has been replaced with a human SIRPα gene and/or a nucleotide sequence that encodes a region of human SIRPα sequence or an amino acid sequence that is at least 10%, 20%, 30%, 40%, 50%, 60%, 70&, 80%, 90%, 95%, 96%, 97%, 98%, or 99% identical to the human SIRPα sequence. In various embodiments, an endogenous non-human SIRPα locus is modified in whole or in part to comprise human nucleic acid sequence encoding at least one protein-coding sequence of a mature SIRPα protein.

In some embodiments, the genetically modified mice express the human SIRPα and/or chimeric SIRPα (e.g., humanized SIRPα) from endogenous loci that are under control of mouse promoters and/or mouse regulatory elements. The replacement(s) at the endogenous mouse loci provide non-human animals that express human SIRPα or chimeric SIRPα (e.g., humanized SIRPα) in appropriate cell types and in a manner that does not result in the potential pathologies observed in some other transgenic mice known in the art. The human SIRPα or the chimeric SIRPα (e.g., humanized SIRPα) expressed in animal can maintain one or more functions of the wildtype mouse or human SIRPα in the animal. For example, SIRPα can bind to human or non-human CD47, and downregulate immune response, e.g., downregulate immune response by at least 10%, 20%, 30%, 40%, or 50%. Furthermore, in some embodiments, the animal does not express endogenous SIRPα. As used herein, the term “endogenous SIRPα” refers to SIRPα protein that is expressed from an endogenous SIRPα nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human SIRPα (e.g., SEQ ID NO: 77). In some embodiments, the genome comprises a sequence encoding an amino acid sequence that is at least 70%, 75%, 80%, 85%, 90%, 95%, 99%, or 100% identical to SEQ ID NO: 53.

The genome of the genetically modified animal can comprise a replacement at an endogenous SIRPα gene locus of a sequence encoding a region of endogenous SIRPα with a sequence encoding a corresponding region of human SIRPα. In some embodiments, the sequence that is replaced is any sequence within the endogenous SIRPα gene locus, e.g., exon 1, exon 2, exon 3, exon 4, exon 5, exon 6, exon 7, exon 8, 5′-UTR, 3′UTR, the first intron, the second intron, and the third intron, the fourth intron, the fifth intron, the sixth intron, or the seventh intron etc. In some embodiments, the sequence that is replaced is within the regulatory region of the endogenous SIRPα gene. In some embodiments, the sequence that is replaced is exon 2 or part thereof, of an endogenous mouse SIRPα gene locus.

The genetically modified animal can have one or more cells expressing a human or chimeric SIRPα (e.g., humanized SIRPα) having an extracellular region and a cytoplasmic region, wherein the extracellular region comprises a sequence that is at least 50%, 60%, 70%, 80%, 90%, 95%, 99% identical to the extracellular region of human SIRPα. In some embodiments, the extracellular region of the humanized SIRPα has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to human SIRPα.

Because human SIRPα and non-human SIRPα (e.g., mouse SIRPα) sequences, in many cases, are different, antibodies that bind to human SIRPα will not necessarily have the same binding affinity with non-human SIRPα or have the same effects to non-human SIRPα. Therefore, the genetically modified animal having a human or a humanized extracellular region can be used to better evaluate the effects of anti-human SIRPα antibodies in an animal model. In some embodiments, the genome of the genetically modified animal comprises a sequence encoding an amino acid sequence that corresponds to part or the entire sequence of exon 3 of human SIRPα, part or the entire sequence of the extracellular region of human SIRPα (with or without signal peptide), or part or the entire sequence of amino acids 31-138 of SEQ ID NO: 77.

In some embodiments, the non-human animal can have, at an endogenous SIRPα gene locus, a nucleotide sequence encoding a chimeric human/non-human SIRPα polypeptide, wherein a human portion of the chimeric human/non-human SIRPα polypeptide comprises a portion of human SIRPα extracellular region, and wherein the animal expresses a functional SIRPα on a surface of a cell of the animal. The human portion of the chimeric human/non-human SIRPα polypeptide can comprise a portion of exon 3 of human SIRPα. In some embodiments, the human portion of the chimeric human/non-human SIRPα polypeptide can comprise a sequence that is at least 80%, 85%, 90%, 95%, or 99% identical to amino acids 31-138 of SEQ ID NO: 77.

In some embodiments, the non-human portion of the chimeric human/non-human SIRPα polypeptide comprises the transmembrane region, and/or the cytoplasmic region of an endogenous non-human SIRPα polypeptide. There may be several advantages that are associated with the transmembrane and/or cytoplasmic regions of an endogenous non-human SIRPα polypeptide. For example, once CD47 binds to SIRPα, they can properly transmit extracellular signals into the cells and regulate the downstream pathway. A human or humanized transmembrane and/or cytoplasmic regions may not function properly in non-human animal cells. In some embodiments, a few extracellular amino acids that are close to the transmembrane region of SIRPα are also derived from endogenous sequence.

Furthermore, the genetically modified animal can be heterozygous with respect to the replacement at the endogenous SIRPα locus, or homozygous with respect to the replacement at the endogenous SIRPα locus.

In some embodiments, the humanized SIRPα locus lacks a human SIRPα 5′-UTR. In some embodiment, the humanized SIRPα locus comprises a rodent (e.g., mouse) 5′-UTR. In some embodiments, the humanization comprises a human 3′-UTR. In appropriate cases, it may be reasonable to presume that the mouse and human SIRPα genes appear to be similarly regulated based on the similarity of their 5′-flanking sequence. As shown in the present disclosure, humanized SIRPα mice that comprise a replacement at an endogenous mouse SIRPα locus, which retain mouse regulatory elements but comprise a humanization of SIRPα encoding sequence, do not exhibit obvious pathologies. Both genetically modified mice that are heterozygous or homozygous for humanized SIRPα are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized SIRPα gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized SIRPα in the genome of the animal.

In some embodiments, the non-human mammal comprises the genetic construct as described herein. In some embodiments, a non-human mammal expressing human or humanized SIRPα is provided. In some embodiments, the tissue-specific expression of human or humanized SIRPα protein is provided.

In some embodiments, the expression of human or humanized SIRPα in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human SIRPα protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized SIRPα protein.

In some embodiments, the genetically-modified non-human animal does not express CD132 (e.g., intact or functional CD132 protein). Because CD132 is a cytokine receptor sub-unit that is common to the receptor complexes for IL-2, IL-4, IL-7, IL-9, IL-15 and IL-21, the genetically-modified non-human animal does not have functional IL-2, IL-4, IL-7, IL-9, IL-15 and/or IL-21.

Furthermore, because IL-7 and IL-15 are important for T and NK cell development, and IL-4 and IL-21 are important for B cell development, in some embodiments, the genetically-modified non-human animal lack functional T cells, B cells, and/or NK cells.

Thus, in some embodiments, the animal can have one or more of the following characteristics:

(a) the percentage of T cells (CD3+ cells) is less than 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2% or 0.1% of leukocytes in the animal;

(b) the percentage of B cells (e.g., CD3− CD19+ cells) is less than 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02% or 0.01% of leukocytes in the animal;

(c) the percentage of NK cells (e.g., CD3− CD49b+ cells) is less than 2%, 1.9%, 1.8%, 1.7%, 1.6%, 1.5%, 1.4%, 1.3%, 1.2%, 1.1%, 1%, 0.9%, 0.8%, 0.7%, 0.6%, or 0.5% of leukocytes in the animal;

(d) the percentage of CD4+ T cells (CD3+CD4+ cells) is less than 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, or 0.1% of T cells;

(e) the percentage of CD8+ T cells (CD3+CD8+ cells) is less than 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, or 0.1% of T cells;

(f) the percentage of CD3+CD4+ cells, CD3+CD8+ cells, CD3− CD19+ cells is less than 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, or 0.1% of leukocytes in the animal;

(g) the percentage of T cells (CD3+ cells) and NK cells (CD3− CD49b+ cells) is less than 5%, 4%, 3%, 2%, 1%, 0.9%, 0.8%, 0.7%, 0.6%, 0.5%, 0.4%, 0.3%, 0.2%, or 0.1% of leukocytes in the animal.

As used herein, the term “leukocytes” or “white blood cells” include neutrophils, eosinophils (acidophilus), basophils, lymphocytes, and monocytes. All leukocytes have nuclei, which distinguishes them from the a nucleated red blood cells (RBCs) and platelets. CD45, also known as leukocyte common antigen (LCA), is a cell surface marker for leukocytes. Among leukocytes, monocytes and neutrophils are phagocytic.

Lymphocytes is a subtype of leukocytes. Lymphocytes include natural killer (NK) cells (which function in cell-mediated, cytotoxic innate immunity), T cells, and B cells.

In some embodiments, the variations among individual B-NDG mice are very small. In some embodiments, the standard deviations of the percentages are less than 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02% or 0.01%.

In some embodiments, the genetically-modified non-human animal is a mouse. The genetically-modified mouse can also have one or more of the following characteristics:

(a) the genetically-modified mouse has no functional T-cells and/or no functional B-cells;

(b) the genetically-modified mouse exhibits reduced macrophage function relative to a NOD/scid mouse, or a NOD/scid nude mouse;

(c) the genetically-modified mouse exhibits no NK cell activity;

(d) the genetically-modified mouse exhibits reduced dendritic function relative to a NOD/scid mouse, or a NOD/scid nude mouse; and

(e) the genetically-modified mouse has an enhanced engraftment capacity of exogenous cells relative to a NOD/scid mouse, or a NOD/scid nude mouse.

### Vectors

The present disclosure relates to a targeting vector, comprising: a) a DNA fragment homologous to the 5′ end of a region to be altered (5′ homologous arm), which is selected from the SIRPα gene genomic DNAs in the length of 100 to 10,000 nucleotides; b) a desired/donor DNA sequence encoding a donor region; and c) a second DNA fragment homologous to the 3′ end of the region to be altered (3′ homologous arm), which is selected from the SIRPα gene genomic DNAs in the length of 100 to 10,000 nucleotides.

In some embodiments, a) the DNA fragment homologous to the 5′ end of a conversion region to be altered (5′ homologous arm) is selected from the nucleotide sequences that have at least 90% homology to the NCBI accession number NC_000068.7; c) the DNA fragment homologous to the 3′ end of the region to be altered (3′ homologous arm) is selected from the nucleotide sequences that have at least 90% homology to the NCBI accession number NC_000068.7.

In some embodiments, a) the DNA fragment homologous to the 5′ end of a region to be altered (5′ homologous arm) is selected from the nucleotides from the position 129607346 to the position 129608914 of the NCBI accession number NC_000068.7; c) the DNA fragment homologous to the 3′ end of the region to be altered (3′ homologous arm) is selected from the nucleotides from the position 129609239 to the position 129610638 of the NCBI accession number NC_000068.7.

In some embodiments, the length of the selected genomic nucleotide sequence in the targeting vector can be about 3 kb, about 3.5 kb, about 4 kb, about 4.5 kb, or about 5 kb.

In some embodiments, the region to be altered is exon 1, exon 2, exon 3, exon 4, exon 5, exon 6, exon 7, or exon 8 of SIRPα gene (e.g., exon 2 of mouse SIRPα gene).

The targeting vector can further include a selected gene marker.

In some embodiments, the sequence of the 5′ homologous arm is shown in SEQ ID NO: 16; and the sequence of the 3′ homologous arm is shown in SEQ ID NO: 17.

In some embodiments, the sequence is derived from human (e.g., 1915110-1915433 of NC_000020.11). For example, the target region in the targeting vector is a part or entirety of the nucleotide sequence of a human SIRPα, preferably exon 3 of the human SIRPα. In some embodiments, the nucleotide sequence of the humanized SIRPα encodes the entire or the part of human SIRPα protein (e.g., SEQ ID NO: 77).

The disclosure also relates to a cell comprising the targeting vectors as described above. In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

The disclosure also provides vectors for constructing a CD132 animal model. In some embodiments, the vectors comprise sgRNA sequence, wherein the sgRNA sequence target CD132 gene, and the sgRNA is unique on the target sequence of the CD132 gene to be altered, and meets the sequence arrangement rule of 5′-NNN (20)-NGG3′ or 5′-CCN—N(20)-3′; and in some embodiments, the targeting site of the sgRNA in the mouse CD132 gene is located on the exon 1, exon 2, exon 3, exon 4, exon 5, exon 6, exon 7, exon 8, intron 1, intron 2, intron 3, intron 4, intron 5, intron 6, and intron 7, upstream of exon 1, or downstream of exon8 of the mouse CD132 gene.

In some embodiments, the 5′ targeting sequence for the knockout sequence is shown as SEQ ID NOS: 1-4, and the sgRNA sequence recognizes the 5′ targeting site. In some embodiments, the 3′ targeting sequence for the knockout sequence is shown as SEQ ID NOS: 5-8 and the sgRNA sequence recognizes the 3′ targeting site.

Thus, the disclosure provides sgRNA sequences for constructing a CD132 knockout animal model. In some embodiments, the oligonucleotide sgRNA sequences are set forth in SEQ ID NOS: 9-12.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous SIRPα gene locus, a sequence encoding a region of an endogenous SIRPα with a sequence encoding a corresponding region of human or chimeric SIRPα. In some embodiments, the replacement occurs in a germ cell, a somatic cell, a blastocyst, or a fibroblast, etc. The nucleus of a somatic cell or the fibroblast can be inserted into an enucleated oocyte.

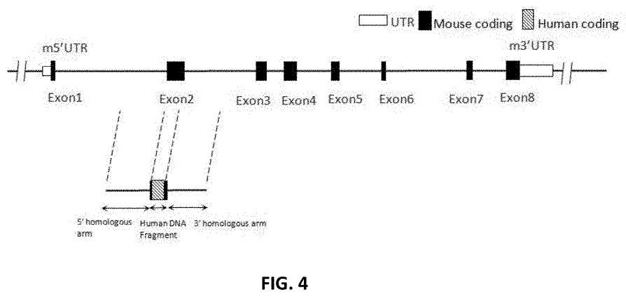

FIG. 4 shows a humanization strategy for a mouse SIRPα locus. In FIG. 4, the targeting strategy involves a vector comprising the 5′ end homologous arm, human SIRPα gene fragment, 3′ homologous arm. The process can involve replacing endogenous SIRPα sequence with human sequence by homologous recombination. In some embodiments, the cleavage at the upstream and the downstream of the target site (e.g., by zinc finger nucleases, TALEN or CRISPR) can result in DNA double strand break, and the homologous recombination is used to replace endogenous SIRPα sequence with human SIRPα sequence.

Thus, in some embodiments, the methods for making a genetically modified, humanized animal, can include the step of replacing at an endogenous SIRPα locus (or site), a nucleic acid encoding a sequence encoding a region of endogenous SIRPα with a sequence encoding a corresponding region of human SIRPα. The sequence can include a region (e.g., a part or the entire region) of exon 1, exon 2, exon 3, exon 4, exon 5, exon 6, exon 7, exon 8, and/or exon 9 of a human SIRPα gene. In some embodiments, the sequence includes a region of exon 3 of a human SIRPα gene (e.g., amino acids 31-138 of SEQ ID NO: 77). In some embodiments, the region is located within the extracellular region of SIRPα. In some embodiments, the endogenous SIRPα locus is exon 2 of mouse SIRPα.

In some embodiments, the methods of modifying a SIRPα locus of a mouse to express a chimeric human/mouse SIRPα peptide can include the steps of replacing at the endogenous mouse SIRPα locus a nucleotide sequence encoding a mouse SIRPα with a nucleotide sequence encoding a human SIRPα, thereby generating a sequence encoding a chimeric human/mouse SIRPα.

In some embodiments, the nucleotide sequence encoding the chimeric human/mouse SIRPα can include a first nucleotide sequence encoding a region of the extracellular region of mouse SIRPα (with or without the mouse or human signal peptide sequence); a second nucleotide sequence encoding a region of the extracellular region of human SIRPα; a third nucleotide sequence encoding the transmembrane region, and/or the cytoplasmic region of a mouse SIRPα.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a SIRPα gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 or BALB/c mouse).

In some embodiments, the non-human mammal in step (c) is a female with pseudopregnancy (or false pregnancy).

In some embodiments, the fertilized eggs for the methods described above are C57BL/6 or BALB/c fertilized eggs. Other fertilized eggs that can also be used in the methods as described herein include, but are not limited to, FVB/N fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the method described above.

Thus, in some embodiments, the disclosure provides knocking out in at least one cell of the animal, at an endogenous CD132 gene locus, one or more exons (e.g., 1, 2, 3, 4, 5, 6, 7, or 8 exons) and/or one or more introns (e.g., 1, 2, 3, 4, 5, 6, or 7 introns) of the endogenous CD132 gene. In some embodiments, the modification occurs in a germ cell, a somatic cell, a blastocyst, or a fibroblast, etc. The nucleus of a somatic cell or the fibroblast can also be inserted into an enucleated oocyte.

The present disclosure further provides a method for establishing a CD132 gene knockout animal model, involving the following steps:

(a) providing the cell (e.g. a fertilized egg cell) with the genetic modification based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse, a NOD/scid mouse, or a NOD/scid nude mouse). In some embodiments, the non-human mammal is a NOD/scid mouse. In the NOD/scid mouse, the SCID mutation has been transferred onto a non-obese diabetic (NOD) background. Animals homozygous for the SCID mutation have impaired T and B cell lymphocyte development. The NOD background additionally results in deficient natural killer (NK) cell function. In some embodiments, the non-human mammal is a NOD/scid nude mouse. The NOD/scid nude mouse additionally has a disruption of FOXN1 gene on chromosome 11 in mice.

In some embodiments, the fertilized eggs for the methods described above are NOD/scid fertilized eggs or NOD/scid nude fertilized eggs. Other fertilized eggs that can also be used in the methods as described herein include, but are not limited to, C57BL/6 fertilized eggs, FVB/N fertilized eggs, BALB/c fertilized eggs, DBA/1 fertilized eggs and DBA/2 fertilized eggs.

The genetically modified animals (e.g., mice) as described herein can have several advantages. For example, the genetically modified mice do not require backcrossing, and thus have a relatively purer background (e.g., NOD/scid) as compared to some other immunodeficient mice known in the art. A pure background is beneficial to obtain consistent experiment results. Furthermore, because almost all sequences in CD132 have been knocked out, these mice are likely to have a higher degree of immunodeficiency and are likely to be better recipients for engraftment as compared to some other immunodeficient mice known in the art. Despite the immunodeficiency, these mice are also relatively healthy, and have a relatively long life span (e.g., more than 1 year, 1.5 years, or 2 years).

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized SIRPα protein, e.g., in a physiologically appropriate manner, provide a variety of uses that include, but are not limited to, developing therapeutics for human diseases and disorders, and assessing the toxicity and/or efficacy of these human therapeutics in the animal models.

In various aspects, genetically modified animals are provided that express human or humanized SIRPα, which are useful for testing agents that can decrease or block the interaction between SIRPα and CD47 or the interaction between SIRPα and other SIRPα receptors or ligands (e.g., surfactant protein A and D), testing whether an agent can increase or decrease the immune response, and/or determining whether an agent is an SIRPα agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of an anti-SIRPα antibody (or an anti-CD47 antibody) for the treatment of cancer. The methods involve administering the anti-SIRPα antibody (or an anti-CD47 antibody) to the animal as described herein, wherein the animal has a tumor; and determining the inhibitory effects of the anti-SIRPα antibody (or an anti-CD47 antibody) to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. In some embodiments, the anti-SIRPα antibody (or an anti-CD47 antibody) has a functional Fc. In some embodiments, the anti-SIRPα antibody (or an anti-CD47 antibody) does not have a functional Fc.

In some embodiments, the tumor comprises one or more cancer cells (e.g., human or mouse cancer cells) that are injected into the animal. In some embodiments, the anti-SIRPα antibody or anti-CD47 antibody prevents CD47 from binding to SIRPα. In some embodiments, the anti-SIRPα antibody or anti-CD47 antibody cannot prevent CD47 from binding to SIRPα (e.g., endogenous SIRPα).

In some embodiments, the genetically modified animals can be used for determining effectiveness of anti-SIRPα antibody (or an anti-CD47 antibody) and one or more additional therapeutic agents for the treatment of cancer.

In some embodiments, the genetically modified animals can be used for determining whether an anti-SIRPα antibody is a SIRPα agonist or antagonist. In some embodiments, the methods as described herein are also designed to determine the effects of the agent (e.g., anti-SIRPα antibodies) on SIRPα, e.g., whether the agent can stimulate macrophages, whether the agent can initiate an antitumor T-cell immune response, and/or whether the agent can upregulate the immune response or downregulate immune response. In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-SIRPα antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-SIRPα antibody or anti-CD47 antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the antibody is designed for treating hepatocellular, ovarian, colon, or cervical carcinomas. In some embodiments, the antibody is designed for treating advanced breast cancer, advanced ovarian cancer, and/or advanced refractory solid tumor. In some embodiments, the antibody is designed for treating metastatic solid tumors, NSCLC, melanoma, non-Hodgkin lymphoma, colorectal cancer, and multiple myeloma. In some embodiments, the treatment is designed for treating acute myeloid leukemia, non-Hodgkin's lymphoma, bladder cancer, or breast cancer.

In some embodiments, the antibody is designed for treating various autoimmune diseases. Thus, the methods as described herein can be used to determine the effectiveness of an antibody in inhibiting immune response.

The present disclosure also provides methods of determining toxicity of an antibody (e.g., anti-SIRPα antibody or anti-CD47 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the SIRPα gene function, human SIRPα antibodies, drugs for human SIRPα targeting sites, the drugs or efficacies for human SIRPα targeting sites, the drugs for immune-related diseases and antitumor drugs.

Genetically modified animals with a disruption at endogenous CD132 gene can provide a variety of uses that include, but are not limited to, establishing a human hemato-lymphoid animal model, developing therapeutics for human diseases and disorders, and assessing the efficacy of these therapeutics in the animal models.

In some embodiments, the genetically modified animals can be used for establishing a human hemato-lymphoid system. The methods involve engrafting a population of cells comprising human hematopoietic cells (CD34+ cells) or human peripheral blood cells into the genetically modified animal described herein. In some embodiments, the methods further include the step of irradiating the animal prior to the engrafting. The human hemato-lymphoid system in the genetically modified animals can include various human cells, e.g., hematopoietic stem cells, myeloid precursor cells, myeloid cells, dendritic cells, monocytes, granulocytes, neutrophils, mast cells, lymphocytes, and platelets.

The genetically modified animals described herein (e.g., with deletion of part of exon 1, deletion of exons 2-7, and deletion of part exon 8) are also an excellent animal model for establishing the human hemato-lymphoid system. In some embodiments, the animal after being engrafted with human hematopoietic stem cells or human peripheral blood cells to develop a human immune system has one or more of the following characteristics:

(a) the percentage of human CD45+ cells is greater than 50%, 55%, 65% 70%, 75%, 80%, 85%, or 90% of leukocytes or CD45+ cells of the animal;

(b) the percentage of human CD3+ cells is greater than 35%, 40%, 45%, 50%, 55%, or 60% of leukocytes or CD45+ cells in the animal; and

(c) the percentage of human CD19+ cells is greater than 15%, 20%, 25%, or 30% of leukocytes or CD45+ cells in the animal.

The genetically modified animals described herein specifically does not include NSG mice or NOG mice, and in some embodiments, are better animal models for establishing the human hemato-lymphoid system (e.g., having a higher percentage of human leukocytes, human T cells, human B cells, or human NK cells). A detailed description of the NSG mice and NOD mice can be found, e.g., in Ishikawa et al. “Development of functional human blood and immune systems in NOD/SCID/IL2 receptor γ chainnull mice.” Blood 106.5 (2005): 1565-1573; Katano et al. “NOD-Rag2null IL-2Rγnull mice: an alternative to NOG mice for generation of humanized mice.” Experimental animals 63.3 (2014): 321-330, both of which are incorporated herein by reference in the entirety.

In some embodiments, the genetically modified animals can be used to determine the effectiveness of an agent or a combination of agents for the treatment of cancer. The methods involve engrafting tumor cells to the animal as described herein, administering the agent or the combination of agents to the animal; and determining the inhibitory effects on the tumors.

In some embodiments, the tumor cells are from a tumor sample obtained from a human patient. These animal models are also known as Patient derived xenografts (PDX) models. PDX models are often used to create an environment that resembles the natural growth of cancer, for the study of cancer progression and treatment. Within PDX models, patient tumor samples grow in physiologically-relevant tumor microenvironments that mimic the oxygen, nutrient, and hormone levels that are found in the patient's primary tumor site. Furthermore, implanted tumor tissue maintains the genetic and epigenetic abnormalities found in the patient and the xenograft tissue can be excised from the patient to include the surrounding human stroma. As a result, PDX models can often exhibit similar responses to anti-cancer agents as seen in the actual patient who provide the tumor sample.

While the genetically modified animals do not have functional T cells or B cells, the genetically modified animals still have functional phagocytic cells, e.g., neutrophils, eosinophils (acidophilus), basophils, or monocytes. Macrophages can be derived from monocytes, and can engulf and digest cellular debris, foreign substances, microbes, cancer cells. Thus, the genetically modified animals described herein can be used to determine the effect of an agent (e.g., anti-CD47 antibodies or anti-SIRPα antibodies) on phagocytosis, and the effects of the agent to inhibit the growth of tumor cells.

In some embodiments, human peripheral blood cells (hPBMC) or human hematopoietic stem cells are injected to the animal to develop human hematopoietic system. The genetically modified animals described herein can be used to determine the effect of an agent in human hematopoietic system, and the effects of the agent to inhibit tumor cell growth or tumor growth. Thus, in some embodiments, the methods as described herein are also designed to determine the effects of the agent on human immune cells (e.g., human T cells, B cells, or NK cells), e.g., whether the agent can stimulate T cells or inhibit T cells, whether the agent can upregulate the immune response or downregulate immune response. In some embodiments, the genetically modified animals can be used for determining the effective dosage of a therapeutic agent for treating a disease in the subject, e.g., cancer, or autoimmune diseases.

In some embodiments, the genetically-modified animals described herein can be injected with human PBMC or CD34+ cells for immune system reconstitution. These animals are useful for efficacy evaluation of anti-human monoclonal antibodies, bispecific antibodies, or combination of drugs; drug screening; or human CAR-T in vivo evaluation or screening.

In some embodiments, the tested agent can be one or more agents selected from the group consisting of paclitaxel, cisplatin, carboplatin, pemetrexed, 5-FU, gemcitabine, oxaliplatin, docetaxel, and capecitabine.

In some embodiments, the tested agent can be an antibody, for example, an antibody that binds to CD47, PD-1, CTLA-4, LAG-3, TIM-3, BTLA, PD-L1, 4-1BB, CD27, CD28, CD47, TIGIT, CD27, GITR, or OX40. In some embodiments, the antibody is a human antibody.

In some embodiments, the genetically-modified animals described herein with a B-NDG background have significantly reduced percentages of T cells (e.g., CD4+ or CD8+ T cells), B cells, and/or NK cells as compared to those in wildtype mice (e.g., C57BL/6 mice). Therefore, the effects of other immune cells, e.g., granulocytes, DC cells, macrophages, and/or monocytes, can be tested with relatively low interference from T cells, B cells, and/or NK cells in the genetically-modified animals described herein with a B-NDG background. It is known in the art that SIRPα is expressed mainly on macrophages, granulocytes, and DC cells. Therefore, the effectiveness (e.g., effectiveness for treating cancer) of an agent targeting the CD47/SIRPα axis (e.g., an anti-SIRPα antibody or anti-CD47 antibody) can be more significant as compared to that when a genetically-modified animal without B-NDG background (e.g., humanized SIRPα mice with C57BL/6 background, humanized CD47 mice with C57BL/6 background, or double-humanized SIRPα/CD47 mice with C57BL/6 background) is used. Above all, the genetically-modified animals described herein with a B-NDG background (e.g., the B-NDG_hSIRPα/CD47 mice, the B-NDG hCD47 mice, or the B-NDG_hSIRPα/hCD47 mice) provide an animal model for evaluating the in vivo efficacy of an agent targeting the CD47/SIRPα axis (e.g., an anti-SIRPα antibody or anti-CD47 antibody).

Genetically Modified Animal Model with Two or More Human or Chimeric Genes

The present disclosure further relates to methods for generating genetically modified animal model with two or more human or chimeric genes. The animal can comprise a human or chimeric SIRPα gene and a sequence encoding one or more additional human or chimeric protein.

In some embodiments, the additional human or chimeric protein can be CD47, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, or TNF Receptor Superfamily Member 4 (TNFRSF4 or OX40).

In some embodiments, the additional human or chimeric protein is CD47. The animal can have a human or chimeric SIRPα gene as described herein and a human or chimeric CD47 gene as described herein. The animal can be used to determine the toxicities and the efficacy of an anti-SIRPα antibody or an anti-CD47 antibody at the same time. In some embodiments, one or more exons of CD47 are replaced by human sequences. In some embodiments, the replaced CD47 region is exon 2 of the endogenous mouse CD47 gene.

The methods of generating genetically modified animal model with two or more human or chimeric genes (e.g., humanized genes) can include the following steps:

(a) using the methods of introducing human SIRPα gene or chimeric SIRPα gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, CTLA-4, LAG-3, BTLA, PD-L1, CD27, CD28, TIGIT, TIM-3, GITR, OX40, CD137, or CD47. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/117984, PCT/CN2017/120388; each of which is incorporated herein by reference in its entirety.

In some embodiments, the SIRPα humanization is directly performed on a genetically modified animal having a human or chimeric CD47, PD-1, CTLA-4, BTLA, PD-L1, CD27, CD28, TIGIT, TIM-3, GITR, CD137, or OX40 gene.

In some embodiments, the SIRPα humanization is directly performed on a genetically modified animal having a human or chimeric CD47.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-SIRPα antibody and an additional therapeutic agent for the treatment of cancer. The methods include administering the anti-SIRPα antibody and the additional therapeutic agent to the animal, wherein the animal has a tumor; and determining the inhibitory effects of the combined treatment to the tumor. In some embodiments, the additional therapeutic agent is an antibody that specifically binds to CD47, PD-1, CTLA-4, BTLA, PD-L1, CD27, CD28, TIGIT, TIM-3, GITR, CD137, or OX40. In some embodiments, the additional therapeutic agent is an anti-CTLA4 antibody (e.g., ipilimumab), an anti-CD20 antibody (e.g., rituximab), an anti-EGFR antibody (e.g., cetuximab), and an anti-CD319 antibody (e.g., elotuzumab), or anti-PD-1 antibody (e.g., nivolumab).

In some embodiments, the additional therapeutic agent is an additional antibody (e.g., an anti-CD20 antibody) that targets a cancer antigen. In some embodiments, the additional antibody can activate antibody-dependent cellular cytotoxicity (ADCC) or antibody-dependent cellular phagocytosis (ADCP), which aids tumor cell destruction. In some embodiments, the additional antibody has a functional Fc. In some embodiments, the additional antibody does not have a functional Fc.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD47, CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the treatment is designed for treating acute myeloid leukemia, non-Hodgkin's lymphoma, bladder cancer, and breast cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

NOD/scid mice were purchased from Beijing HFK Bioscience Co., Ltd.

T4 DNA Ligase was purchased from Takara Bio Inc. The catalog number is 2011A.

Ambion™ in vitro transcription kit was purchased from Ambion, Inc. The catalog number is AM1354.

E. coli TOP10 competent cells were purchased from the Tiangen Biotech (Beijing) Co. Ltd. The catalog number is CB104-02.

pHSG299 plasmid was purchased from Takara Bio Inc. The catalog number is 3299.

KOD enzyme was purchased from Toyobo Co., Ltd. The catalog number is KOD-101.

Cas9 mRNA was obtained from SIGMA. The catalog number is CAS9MRNA-1EA.

UCA kit was obtained from Beijing Biocytogen Co., Ltd. The catalog number is BCG-DX-001.

EcoRI, NheI, XhoI, NotI, and NdeI were purchased from NEB. The catalog numbers are R3101M, R3131S, R0146S, R3189S, and R0111S respectively.

AIO kit was obtained from Beijing Biocytogen Co. Ltd. The catalog number is BCG-DX-004.

APC/Cy7 anti-mouse TCRβ chain was purchased from BioLegend. The catalog number is 109220.

FITC anti-mouse CD19 was purchased from BioLegend. The catalog number is 115506.

PE anti-mouse CD172a (mSIRPα) was purchased from BioLegend. The catalog number is 144012.

APC anti-human antibody (hSIRPα) was purchased from BioLegend. The catalog number is 323810.

BD Horizon™ V450 Rat Anti-mouse CD11b was purchase from BD Biociences. The catalog number is 560455.

Brilliant Violet 510™ anti-mouse CD45 was purchased from BioLegend. The catalog number is 103138.

PE/Cy™ 7 Mouse anti-mouse NK1.1 was purchased from BioLegend. The catalog number is 552878.

APC anti-mouse CD11c was purchased from BioLegend. The catalog number is 117310.

Brilliant Violet711™ anti-mouse TCRβ Chain was purchased from BioLegend. The catalog number is 109243.

PE anti-mouse CD19 was purchased from BioLegend. The catalog number is 115508.

PerCP anti-mouse Ly-6G/Ly-6C (Gr-1) antibody was purchased from BioLegend. The catalog number is 108426.

FITC anti-mouse F4/80 was purchased from BioLegend. The catalog number is 123108.

Purified anti-mouse CD16/32 was purchased from BioLegend. The catalog number is 101302.

Flow cytometer was purchased from BD Biosciences (model: FACS Calibur™)

### Example 1: Preparation of CD132 Gene Knockout Mice

The target sequence determines the targeting specificity of small guide RNA (sgRNA) and the efficiency of Cas9 cleavage at the target site. Therefore, target sequence selection is important for sgRNA vector construction.

The mice used in the examples had a NOD/scid background.

Several sgRNAs were designed for the mouse CD132 gene. The target sequences for these sgRNAs are shown below:

sgRNA-1 targeting site (SEQ ID NO: 1):
5′-CCACCGGAAGCTACGACAAAAGG-3′
sgRNA-2 targeting site (SEQ ID NO: 2):
5′-TCTCTACAGCGTGGTTTCTAAGG-3′
sgRNA-3 targeting site (SEQ ID NO: 3):
5′-GGCTTGTGGGAGAGTGGTTCAGG-3′
sgRNA-4 targeting site (SEQ ID NO: 4):
5′-CCACGCTGTAGAGAGAGGGGGGG-3′
sgRNA-5 targeting site (SEQ ID NO: 5):
5′-AGGGGAGGTTAGCGTCACTTAGG-3′
sgRNA-6 targeting site (SEQ ID NO: 6):
5′-GAAATCGAAACTTAGCCCCAAGG-3′
sgRNA-7 targeting site (SEQ ID NO: 7):
5′-GCAGCCTGCATAGCCCTTACTGG-3′
sgRNA-8 targeting site (SEQ ID NO: 8):
5′-CCCTACTCACCTTGGCAATCTGG-3′

sgRNA-1, sgRNA-2, sgRNA-3, and sgRNA-4 target the 5′-end targeting sites and sgRNA-5, sgRNA-6, sgRNA-7, and sgRNA-8 target the 3′-end targeting sites. Among them, the targeting sites for sgRNA-1, sgRNA-2, and sgRNA-4 are located upstream of exon 1 of the mouse endogenous CD132 gene (Gene ID: 16186). The targeting site for sgRNA-3 is located on exon 1 of mouse endogenous CD132 gene. The target sites for sgRNA-5, sgRNA-6, sgRNA-7, and sgRNA-8 are all located on exon 8 of the mouse endogenous CD132 gene.

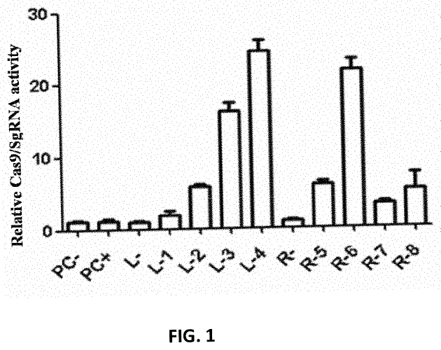

The UCA kit was used to detect the activities of sgRNAs, L-1 to L-4 correspond to sgRNA-1 to sgRNA-4, respectively. R-5 to R-8 correspond to sgRNA5 to sgRNA8, respectively. As shown in FIG. 1, the results showed that the sgRNAs had different activities. In particular, sgRNA-1 and sgRNA-7 exhibited relatively low activities, which may be caused by sequence variations. However, the relative activities of sgRNA-1 and sgRNA-7 were still significantly higher than that of the negative control (PC−). Therefore, sgRNA-1 and sgRNA-7 can be used for the gene editing experiment as well. sgRNA-3 and sgRNA-6 were randomly selected for subsequent experiments.

The forward oligonucleotide (F) sequences and reverse oligonucleotide (R) sequences of sgRNA-3 and sgRNA-6 are as follows:

(SEQ ID NO: 9)
sgRNA-3 F: 5′-CACCGGCTTGTGGGAGAGTGGTTC-3′
(SEQ ID NO: 10)
sgRNA-3 R: 5′-AAACGAACCACTCTCCCACAAGCC-3′
(SEQ ID NO: 11)
sgRNA-6 F: 5′-CACCGGAAATCGAAACTTAGCCCCA-3′
(SEQ ID NO: 12)
sgRNA-6 R: 5′-AAACTGGGGCTAAGTTTCGATTTCC-3′

CACC or CACCG were added to the 5′ end of the upstream and downstream single strands of sgRNA-3 and sgRNA-6 to obtain forward oligonucleotide sequences. AAAC was added to the 5′ end of the complementary strands to obtain reverse oligonucleotide sequences. The sgRNA forward and reverse oligonucleotide sequences were obtained by gene synthesis.

Then, the pT7-sgRNA-G2 plasmid containing T7 promoter and sgRNA scaffold was synthesized. The DNA fragment containing the T7 promoter and sgRNA scaffold is shown below:

(SEQ ID NO: 13)
GAATTCTAATACGACTCACTATAGGGGGTCTTCGAGAAGACCTGTTTTAG
AGCTAGAAATAGCAAGTTAAAATAAGGCTAGTCCGTTATCAACTTGAAAA
AGTGGCACCGAGTCGGTGCTTTTAAAGGATCC

After annealing the forward and reverse oligonucleotides of sgRNA-3 and sgRNA-6, the product was ligated to the pT7-sgRNA plasmid. The ligation reaction system is shown in the table below.

TABLE 10
The ligation reaction conditions (10 μL)

| | |
|---|---|
| sgRNA annealing product | 1 μL (0.5 μM) |
| pT7-sgRNA vector | 1 uL (10 ng) |
| T4 DNA Ligase | 1 μL (5U) |
| 10 × T4 DNA Ligase buffer | 1 μL |
| 50% PEG4000 | 1 μL |
| H₂O | Add to 10 μL |

The ligation reaction was carried out at room temperature for 10-30 minutes. The ligation product was then transferred to 30 μL of TOP10 competent cells. The cells were then plated on a petri dish with Kanamycin, and then cultured at 37° C. for at least 12 hours and then clones were selected and added to LB medium with Kanamycin (5 ml), and then cultured at 37° C. at 250 rpm for at least 12 hours.

Clones were randomly selected and sequenced to verify their sequences. The pT7-IL-3 and pT7-IL-6 vectors with correct sequences were selected for subsequent experiments.

The pre-mixed Cas9 mRNA, in vitro transcription products of pT7-IL-3 and pT7-IL-6 plasmids were injected into the cytoplasm or nucleus of NOD/scid mouse fertilized eggs with a microinjection instrument (using Ambion in vitro transcription kit to carry out the transcription according to the method provided in the product instruction). The embryo microinjection was carried out according to the method described, e.g., in A. Nagy, et al., “Manipulating the Mouse Embryo: A Laboratory Manual (Third Edition),” Cold Spring Harbor Laboratory Press, 2006.

The injected fertilized eggs were then transferred to a culture medium to culture for a short time and then was transplanted into the oviduct of the recipient mouse to produce the genetically modified mice (F0 generation). The mice population was further expanded by cross-breeding and self-breeding to establish stable mouse lines. These genetically modified mouse model (CD132 knockout) was named as B-NDG mouse.

### Example 2. Verification of CD132 Gene Knockout Mice

Mouse tail genomic DNA was used to verify the genotype of the CD132 gene knockout mice obtained in Example 1. The PCR reaction system and reaction conditions are shown in the tables below.

TABLE 11
The PCR reaction system (20 μL)

| Reagent | Volume | |
|---|---|---|
| 10x KOD buffer | 2 | μL |
| dNTP (2 mM) | 2 | μL |
| MgSO₄ (25 mM) | 0.8 | μL |
| Upstream primer (10 μM) | 0.6 | μL |
| Downstream primer (10 μM) | 0.6 | μL |
| Mouse tailgenomic DNA (gDNA) | 200 | ng |
| KOD (200U) | 0.6 | μL |

| | |
|---|---|
| H₂O | Add to 20 μL |

TABLE 12
The PCR reaction conditions

| Temperature | Time | Cycles |
|---|---|---|
| 95° C. | 5 min | 1 |
| 95° C. | 30 sec | 15 |
| 67° C. | 30 sec | |
| 72° C. | 1 kb/min | |
| 72° C. | 10 min | 1 |
| 4° C. | 10 min | 1 |

The PCR primers are shown below:

(SEQ ID NO: 14)
PCR-1F: 5′-AAGATAGCCTAGAGGGAAAAGGTGG-3′
(SEQ ID NO: 15)
PCR-1R: 5′-AGGTAGAAAAAGGGAGGGAGAATCC-3′

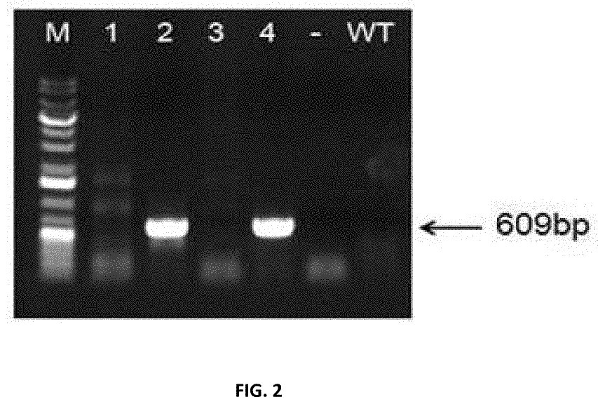

The mouse tail genomic DNAPCR results are shown in FIG. 2. The size of the PCR amplification products from No. 2 and No. 4 mice was 609 bp, which was consistent with the predicted size. Thus, No. 2 and No. 4 mice were identified as positive mice, i.e., B-NDG mice.

### Example 3. Analysis of the Expression of CD132 Gene Knockout Mice

Three CD132 gene knockout mice (B-NDG mice) identified as positive in Example 2, and three NOD/scid mice (control) were selected for subsequence experiments. The spleens of these mice were collected after euthanasia, and the spleen samples were grinded. The grinded samples were then filtered through a 70 μm cell strainer. The filtered cell suspension was centrifuged and supernatant was discarded. Red blood cell lysis solution was then added to the cell sample. The cells were lysed for 5 minutes, and then the lysis reaction was neutralized by PBS solution. Afterwards, the cell sample was centrifuged again and supernatant was discarded. The cells were washed with PBS for one more time before FACS analysis.

The ratios of CD3+ T cells, CD3+CD4+ T cells, CD3+CD8+ T cells, CD3-CD19+B cells, and CD3-CD49b+NK cells in the B-NDG mice and control mice were analyzed.

The table below compares the average value of flow cytometry data of T cells, B cells, and NK cells in B-NDG mice and NOD/scid mice. The results showed that the B-NDG mice as prepared herein lacked T cells, B cells, and NK cells. For example, as compared to NOD/scid mice, the ratios of T cells, B cells, and NK cells in B-NDG mice were significantly reduced. The analysis results showed that the B-NDG mice prepared by the method described herein almost lacked T, B, and NK cells completely, which was in line with the characteristics of highly immuno-deficient mice.

TABLE 13
Cell surface marker expressions in B-NDG mice

| | | NOD/scid | B-NDG |
|---|---|---|---|
| | Cell surface markers | (n = 3) | (n = 3) |
| T cells | CD3+ | 0.75% | 0.56% |
| CD4+ T cells | CD3+ CD4+ | 1.64% | 0.07% |
| CD8+ T cells | CD3+ CD8+ | 0.23% | 0.05% |
| B cells | CD3− CD19+ | 0.17% | 0.03% |
| NK cells | CD3− CD49b+ | 13.33% | 1.37% |

### Example 4. Preparation of SIRPα Gene Humanized Mice

A schematic diagram that compares the mouse SIRPα gene (NM_007547.4, SEQ ID NO: 78) and the human SIRPα gene (NM_080792.2, SEQ ID NO: 76) is shown in FIGS. 3A-3B.

The targeting vector is shown in FIG. 4. The targeting vector contains a 5′ homologous arm, a human SIRPα gene fragment (human DNA fragment), and a 3′ homologous arm. Sequence of the 5′ homologous arm is set forth in SEQ ID NO: 16, which is identical to nucleic acids 129607346-129608914 of NCBI reference sequence NC_000068.7. Sequence of the 3′ homologous arm is set forth in SEQ ID NO: 17, which is identical to nucleic acids 129609239-129610638 of NCBI reference sequence NC_000068.7. The human SIRPα gene sequence is set forth in SEQ ID NO: 18. A sequence in human SIRPα exon 2 spanning from 1915110-1915433 of NCBI reference sequence NC_000020.11 was used to replace a sequence in mouse SIRPα exon 2 starting from the nucleic acid residue after the last G of GAGCCACGAGG (SEQ ID NO: 19) and ending at the nucleic acid residue before the first G of GGAACAGAGGTCTATG (SEQ ID NO: 20).

The DNA or BAC library of the CD132 knockout mice (B-NDG mice) identified as positive in Example 2 was used as PCR templates, to amplify and obtain LR (5′ homologous arm) and RR fragments (3′ homologous arm). The human SIRPα gene fragment (SEQ ID NO: 18) was obtained by PCR amplification using human DNA or BAC library as a template. The targeting vector containing the 5′ homologous arm, the 3′ homologous arm, and the human SIRPα gene fragment was ligated to the pClon-4G plasmid provided in the AIO kit, to obtain the vector pClon-4G-SIRPα.

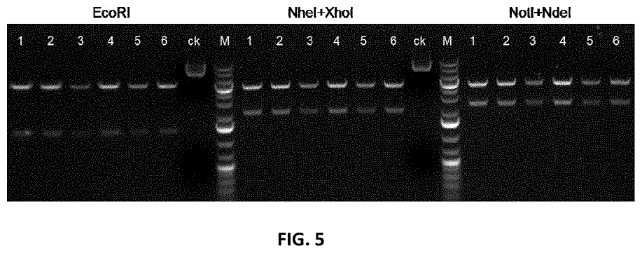

Six pClon-4G-SIRPα clones were randomly selected and tested by five restriction enzymes. Among them, EcoRI digestion should produce 1368 bp+5439 bp fragments. NheI+XhoI digestion should produce 2089 bp+4711 bp fragments. NotI+NdeI digestion should produce 4452 bp+2348 bp fragments. The results are shown in FIG. 5. All six clones showed expected bands with correct molecular weight. The sequences of plasmids 3 and 6 were further verified by sequencing. Plasmid 3 was verified to have correct sequence and was used in the following experiments.

The 5′-terminal targeting sites (sgRNA-1 to sgRNA-10) and the 3′-terminal targeting sites (sgRNA-11 to sgRNA-21) were designed and synthesized. The 5′-terminal targeting sites and the 3′-terminal targeting sites are located on exon 2 of mouse SIRPα gene. The targeting site sequences on SIRPα are as follows:

sgRNA-1 targeting site (SEQ ID NO: 21):
5′-AGTTCCTTCCCCGTGGCTCCTGG-3′
sgRNA-2 targeting site (SEQ ID NO: 22):
5′-AGCCACGGGGAAGGAACTGAAGG-3′
sgRNA-3 targeting site (SEQ ID NO: 23):
5′-CACCTTCAGTTCCTTCCCCGTGG-3′
sgRNA-4 targeting site (SEQ ID NO: 24):
5′-AAATCAGTGTCTGTTGCTGCTGG-3′
sgRNA-5 targeting site (SEQ ID NO: 25):
5′-CACTTTGACCTCCTTGTTGCCGG-3′
sgRNA-6 targeting site (SEQ ID NO: 26):
5′-TTGACCTCCTTGTTGCCGGTGGG-3′
sgRNA-7 targeting site (SEQ ID NO: 27):
5′-GGGTCCCACCGGCAACAAGGAGG-3′
sgRNA-8 targeting site (SEQ ID NO: 28):
5′-TGTTGCCGGTGGGACCCATTAGG-3′
sgRNA-9 targeting site (SEQ ID NO: 29):
5′-ACTCCTCTGTACCACCTAATGGG-3′
sgRNA-10 targeting site (SEQ ID NO: 30):
5′-CTGTAGATCAACAGCCGGCTTGG-3′
sgRNA-11 targeting site (SEQ ID NO: 31):
5′-CGAAACTGTAGATCAACAGCCGG-3′
sgRNA-12 targeting site (SEQ ID NO: 32):
5′-CTGTTGATCTACAGTTTCGCAGG-3′
sgRNA-13 targeting site (SEQ ID NO: 33):
5′-TCTGAAACATTTCTAATTCGAGG-3′
sgRNA-14 targeting site (SEQ ID NO: 34):
5′-TACTACTAAGAGAAACAATATGG-3′
sgRNA-15 targeting site (SEQ ID NO: 35):
5′-CTGGGGTGACATTACTGATACGG-3′
sgRNA-16 targeting site (SEQ ID NO: 36):
5′-AATGTCACCCCAGCAGATGCTGG-3′
sgRNA-17 targeting site (SEQ ID NO: 37):
5′-GTAGATGCCAGCATCTGCTGGGG-3′
sgRNA-18 targeting site (SEQ ID NO: 38):
5′-CCTGACACAGAAATACAATCTGG-3′
sgRNA-19 targeting site (SEQ ID NO: 39):
5′-CACAGAAATACAATCTGGAGGGG-3′
sgRNA-20 targeting site (SEQ ID NO: 40):
5′-ACAATCTGGAGGGGGAACAGAGG-3′
sgRNA-21 targeting site (SEQ ID NO: 41):
5′-GGAACAGAGGTCTATGTACTCGG-3′

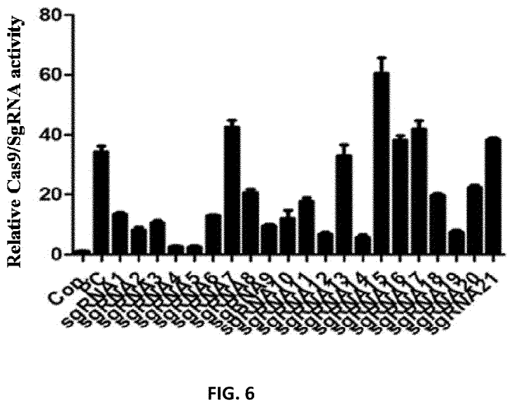

The UCA kit was used to detect the activities of sgRNAs, As shown in FIG. 6, the results showed that the sgRNAs had different activities. In particular, sgRNA-4 and sgRNA-5 exhibited relatively low activities, which may be caused by sequence variations. However, the relative activities of sgRNA-4 and sgRNA-5 were still significantly higher than that of the negative control (Con). Therefore, sgRNA-4 and sgRNA-5 can be used for the gene editing experiment as well sgRNA-7 and sgRNA-17 were randomly selected for subsequent experiments. Specifically, TAGG was added to the 5′ end of the upstream sequence to obtain a forward oligonucleotide sequence, and AAAC was added to the 5′ end of its complementary strand (downstream sequence) to obtain a reverse oligonucleotide sequence. The synthesized sgRNA oligonucleotide sequences based on sgRNA-7 and sgRNA-17 are listed below:

```
sgRNA-7:
Upstream sequence:
(SEQ ID NO: 42)
5′-GTCCCACCGGCAACAAGG-3′
Forward oligonucleotide:
(SEQ ID NO: 43)
5′-TAGGGTCCCACCGGCAACAAGG-3′
Downstream sequence:
(SEQ ID NO: 44)
5′-CCTTGTTGCCGGTGGGAC-3′
Reverse oligonucleotide:
(SEQ ID NO: 45)
5′-AAACCCTTGTTGCCGGTGGGAC-3′
sgRNA-17:
Upstream sequence:
  (SEQ ID NO: 46)
5′-TAGATGCCAGCATCTGCTG-3′
Forward oligonucleotide:
  (SEQ ID NO: 47)
5′-TAGGTAGATGCCAGCATCTGCTG-3′
Downstream sequence:
(SEQ ID NO: 48)
5′-CAGCAGATGCTGGCATCTA-3′
Reverse oligonucleotide:
(SEQ ID NO: 49)
5′-CAGCAGATGCTGGCATCTA-3′
```

Synthesized DNA fragment containing T7 promoter and the sgRNA scaffold (SEQ ID NO: 13) was ligated to the backbone plasmid pHSG299 by restriction enzyme digestion (EcoRI and BamHI). The sequence of the plasmids was confirmed by sequencing.

Sequence of the DNA fragment containing the T7 promoter and sgRNA scaffold is set forth in SEQ ID NO: 13. After annealing the forward and reverse oligonucleotides, the oligonucleotides were ligated to pT7-sgRNA plasmids to produce the expression vectors pT7-sgRNA-S7 and pT7-sgRNA-S17. The ligation reaction is set up as follows:

TABLE 14
The ligation reaction mix (10 μL)

| | |
|---|---|
| sgRNA annealing product | 1 μL (0.5 μM) |
| pT7-sgRNA G2 vector | 1 μL (10 ng) |
| T4 DNA Ligase | 1 μL (5U) |
| 10 × T4 DNA Ligase buffer | 1 μL |
| 50% PEG4000 | 1 μL |
| H₂O | Add to 10 μL |

The ligation reaction was carried out at room temperature for 10-30 minutes. The ligation product was then transferred to 30 μL of TOP10 competent cells. The cells (200 μl) were then plated on a petri dish with Kanamycin, and then cultured at 37° C. for at least 12 hours and then clones were selected and added to LB medium with Kanamycin (5 ml), and then cultured at 37° C. at 250 rpm for at least 12 hours.

Randomly selected clones were sequenced to verify their sequences. The vectors pT7-sgRNA-S7 and pT7-sgRNA-S17 with correct sequences were selected for subsequent experiments.

The pre-mixed Cas9 mRNA, pClon-4G-SIRPα plasmid and in vitro transcription products of pT7-sgRNA-S7, pT7-sgRNA-S17 plasmids were injected into the cytoplasm or nucleus of fertilized eggs from the B-NDG mice (identified as positive in Example 2) with a microinjection instrument (using Ambion in vitro transcription kit to carry out the transcription according to the method provided in the product instruction). The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were then transferred to a culture medium for a short time culture, and then was transplanted into the oviduct of the recipient mouse to produce the genetically modified humanized mice (F0 generation). The mouse population was further expanded by cross-breeding and self-breeding to establish stable mouse lines. The humanized mouse was named as B-NDG_hSIRPα.

### Example 5. Verification of the SIRPα Gene Humanized Mice

The humanized SIRPα gene is shown in FIG. 7. A portion of the humanized SIRPα gene containing the human SIRPα sequence is shown in SEQ ID NO: 50 below:

AGCCACGAGGgaggaggagctgcaggtgattcagcctgacaagtccgtgt
tggttgcagctggagagacagccactctgcgctgcactgcgacctctctg
atccctgtggggcccatccagtggttcagaggagctggaccaggccggga
attaatctacaatcaaaaagaaggccacttcccccgggtaacaactgttt
cagacctcacaaagagaaacaacatggacttttccatccgcatcggtaac
atcaccccagcagatgccggcacctactactgtgtgaagttccggaaagg
gagccccgatgacgtggagtttaagtctggagcaGGAACAGAGGTCTATG

SEQ ID NO: 50 shows only the modified portion of DNA sequence, wherein the italicized underlined region is from human SIRPα.

The coding region sequence, mRNA sequence and the encoded amino acid sequence thereof of the modified humanized SIRPα are respectively set forth in SEQ ID NO: 51, SEQ ID NO: 52, and SEQ ID NO: 53.

Since the human SIRPα gene has multiple transcript variants, the sequence design disclosed herein can be applied to other transcript variants. For example, the following mouse transcript variants and corresponding amino acid sequences of SIRPα can be used:

### NM_001040022.1→NP_001035111.1, NM_001040023.1→NP_001035112.1, NM_001330728.1→NP_001317657.1, XM_005260670.3→XP_005260727.1, XM_006723545.3→XP_006723608.1, and XM_011529173.2→XP_0115274751.

### 1. Genotype Determination for F0 Generation Mice

Two pairs of primers were used to amplify the mouse tail genomic DNA of the F0 generation B-hSIRPA mouse for PCR analysis. Specifically, the primer L-GT-F is on the left side of the 5′ homologous arm, R-GT-R is on the right side of the 3′ homologous arm, and both R-GT-F and L-GT-R are on the humanized fragment. The primer sequences, the PCR reaction system, and the PCR reaction conditions are shown in the tables below.

If the desired human sequence was inserted into the correct positions in the genome, PCR experiments using the primers above should generate only one band. The 5′ end PCR experiment should produce a band at about 2,047 bp, and the 3′ end PCR experiment should produce a band at about 1,836 bp.

TABLE 15
Primer sequences

| Primer | | Product |
|---|---|---|
| name | Sequence (5′-3′) | size (bp) |
| L-GT-F | CATCAAGCCTGTTCCCTCCTTGTGT | Mut: 2047 |
| | (SEQ ID NO: 54) | |
| L-GT-R | CTTAAACTCCACGTCATCGGGGCTC | |
| | (SEQ ID NO: 55) | |
| R-GT-F | TCAAAAAGAAGGCCACTTCCCCCGGG | Mut: 1836 |
| | (SEQ ID NO: 56) | |
| R-GT-R | CAAGCTGTAGAGACAGATGGGCAGG | |
| | (SEQ ID NO: 57) | |

TABLE 16
The PCR reaction system (20 μL)

| | | |
|---|---|---|
| 2x PCR buffer | 10 | μL |
| dNTP (2 mM) | 4 | μL |
| Upstream primer (10 μM) | 0.6 | μL |
| Downstream primer (10 μM) | 0.6 | μL |
| Mouse tail genomic DNA | 100 | ng |
| KOD-FX (1U/μL) | 0.4 | μL |

| | |
|---|---|
| H₂O | Add to 20 μL |

TABLE 17
The PCR reaction conditions

| Temperature | Time | Cycles |
|---|---|---|
| 94° C. | 5 min | 1 |
| 94° C. | 30 sec | 15 |
| 67° C. (−0.7° C./cycle) | 30 sec | |
| 68° C. | 1 kb/min | |
| 94° C. | 30 sec | 25 |
| 56° C. | 30 sec | |
| 68° C. | 1 kb/min | |
| 68° C. | 10 min | 1 |
| 4° C. | 10 min | 1 |

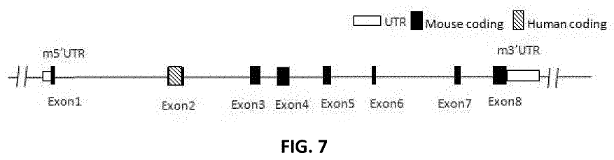

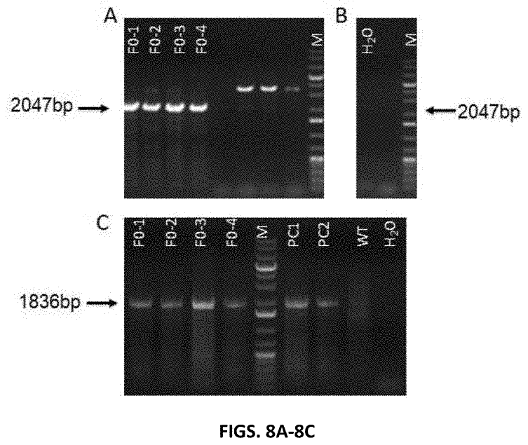

Results are shown in FIGS. 8A-8C. Mice labelled with F0-1, F0-2, F0-3, and F0-4 had PCR products with the correct size. Thus, the four mice were identified as positive mice.

### 2. Genotype Determination for F1 Generation Mice

F1 generation mice were obtained by cross-breeding the F0 generation mice (identified as positive) with CD132 gene knockout mice (identified as positive). PCR experiments were performed using mouse tail genomic DNA from F1 mice. The PCR primers, reaction setup, and conditions were the same as those used in genotyping the F0 generation mice.

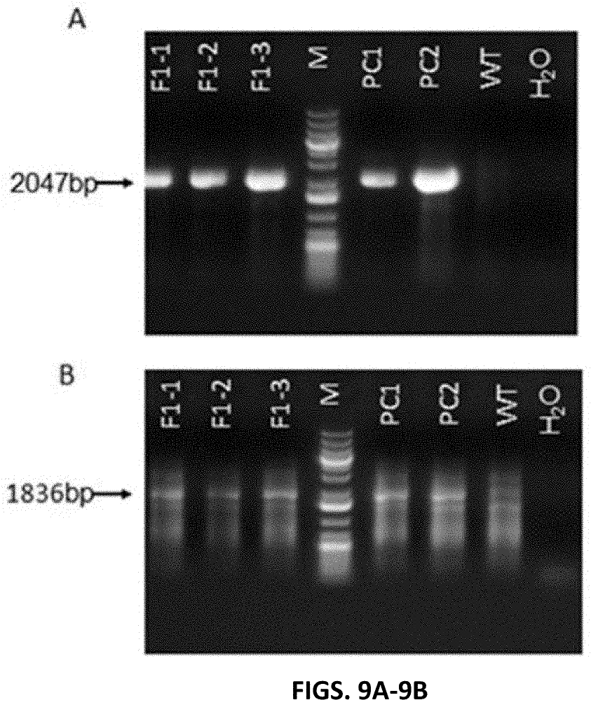

Results are shown in FIGS. 9A-9B. Three randomly selected F1 generation mice F1-1, F1-2, and F1-3 had PCR products with the correct size and thus the human sequences were correctly inserted into the mouse genome.

Furthermore, Southern blot was performed on the three mice to confirm that there was no random insertion. Genomic DNA was extracted from mouse tail, digested with AseI restriction enzyme, blotted, and hybridized with probes P1 and P2. Probes P1 and P2 target to the 5′ homologous arm and on the right side of the 3′ homologous arm, respectively. The primers for synthesizing P1 and P2 probes are as follows:

(SEQ ID NO: 58)
P1-F: 5′-GCCCTCCCCAGCCCCAGATTTTA-3′
(SEQ ID NO: 59)
P1-R: 5′-GAAGTATGCAGATCTCTGTGAT-3′
(SEQ ID NO: 60)
P2-F: 5′-CGCCCACTGTGCAAGGCCTAC-3′
(SEQ ID NO: 61)
P2-R: 5′-GCTCCAAATAGCAAGGTCTTGGG-3′

The hybridization result for a humanized SIRPα mouse should generate a 2.8 kb band and a 3.2 kb band using the P1 and P2 probes, respectively. By contrast, the wildtype mouse should only have a 6.0 kb band.

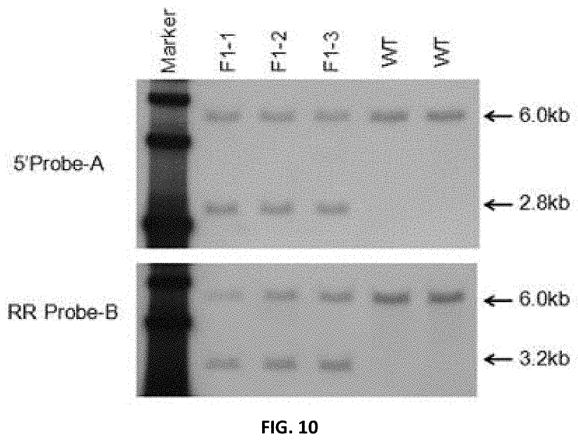

The results of Southern blot detection are shown in FIG. 10. The size of the hybridization bands of the 3 mice were all consistent with expectations, and there was no random insertion. This indicates that the method can be used to construct a humanized immuno-deficient mouse (B-NDG_hSIRPα), that can be passaged stably without random insertion.

### 3. Expression Analysis in Humanized Mice

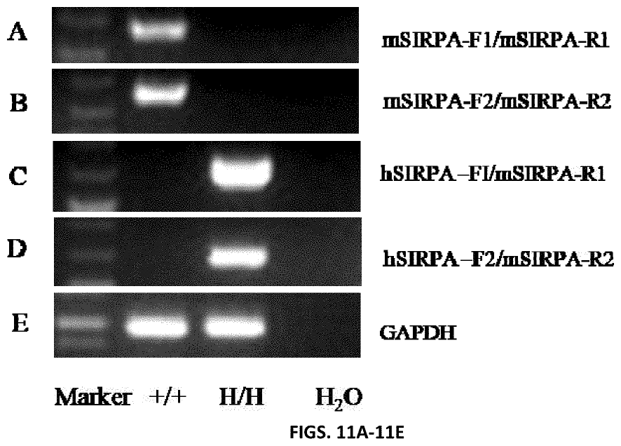

The expression of humanized SIRPα mRNA or protein in positive mice can be confirmed by methods such as RT-PCR, ELISA, etc. RT-PCR was used to detect the humanized SIRPα mRNA in positive mouse spleen, and the results are shown in FIGS. 11A-11E. Only mouse SIRPα mRNA was detected in B-NDG mice (FIGS. 11A-11B), and only humanized SIRPα mRNA was detected in humanized SIRPα homozygous mice (FIGS. 11C-11D). RT-PCR primer sequence as shown in Table 18.

TABLE 18
RT-PCR primer sequence

| | | Product |
|---|---|---|
| Primer | sequence (5′-3′) | size (bp) |
| hSIRPA-F1 | CCCCGATGACGTGGAGTTTAAG | Mut: 723 |
| (SEQ ID NO: 63) | | |
| mSIRPA-R1 | TGGGTAGCATTATTACCAGGGAAGG | |
| (SEQ ID NO: 64) | | |
| hSIRPA-F2 | CCCCGATGACGTGGAGTTTAAGT | Mut: 722 |
| (SEQ ID NO: 65) | | |
| mSIRPA-R2 | GGGTAGCATTATTACCAGGGAAGGT | |
| (SEQ ID NO: 66) | | |
| mSIRPA-F1 | TTGATCTACAGTTTCACAACAGAAC | WT: 878 |
| (SEQ ID NO: 67) | | |
| mSIRPA-R1 | TGGGTAGCATTATTACCAGGGAAGG | |
| (SEQ ID NO: 64) | | |
| mSIRPA-F2 | ACAACAGAACACTTTCCTCGAGTTA | WT: 862 |
| (SEQ ID NO: 68) | | |
| mSIRPA-R2 | GGGTAGCATTATTACCAGGGAAGGT | |
| (SEQ ID NO: 66) | | |
| GAPDH-F | TCACCATCTTCCAGGAGCGAGA | 479 |
| (SEQ ID NO: 69) | | |
| GAPDH-R | GAAGGCCATGCCAGTGAGCTT | |
| (SEQ ID NO: 70) | | |

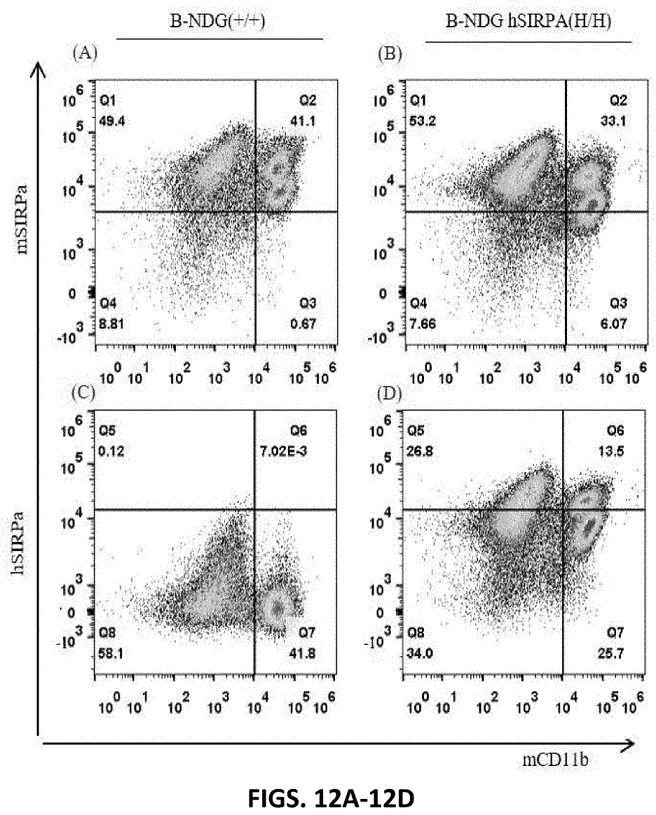

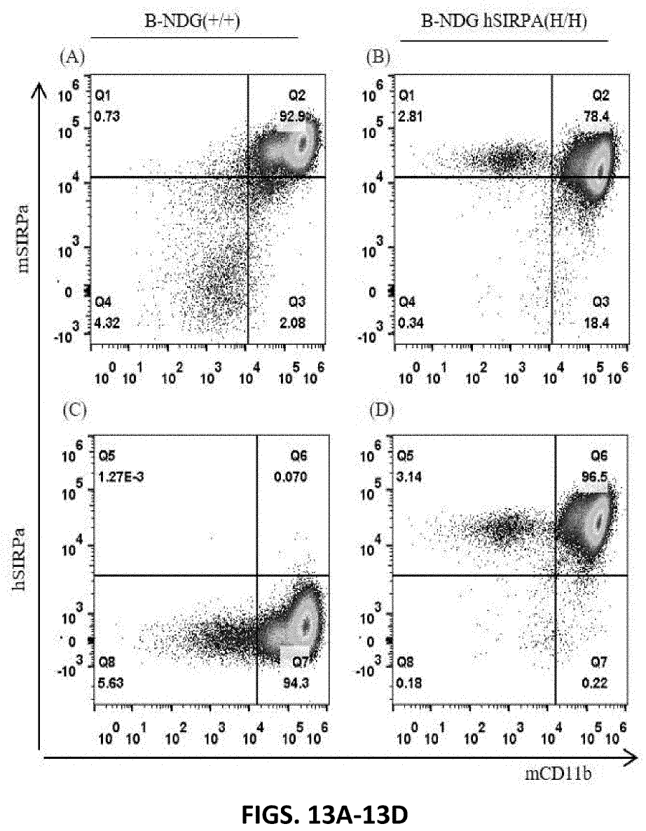

One SIRPα humanized positive homozygous mouse, and one B-NDG mouse with the same background (control) were selected. After the mice were sacrificed, mouse spleen and the abdominal cavity washing fluid (5 mL PBS was intraperitoneally injected after sacrifice) were collected. The spleen was grinded and then passed through a 70 μm cell strainer. The filtered cell suspension was centrifuged and supernatant was discarded. Red blood cell lysis buffer was then added, and PBS solution was added after 5 minutes to neutralize the lysis reaction. The cells were then centrifuged and supernatant was discarded, followed by PBS wash once. Afterwards, mouse SIRPα antibody PE anti-mouse CD172a (mSIRPα), FITC anti-Mouse CD19, APC/Cy7 anti-mouse TCR β chain, APC anti-human antibody (hSIRPα), and V450 Rat anti-mouse CD11b were used for staining SIRPα and cell surface markers. All the cells were washed by PBS once, and flow cytometry was then used to detect the expression of SIRPα protein. The results are shown in FIGS. 12A-12D and 13A-13D. The mouse SIRPα antibody detected the spleen cells expressing the mouse SIRPα protein in the spleen of B-NDG mice and B-NDG_hSIRPα homozygous mice (FIGS. 12A-12B), indicating that the mouse SIRPα antibody may cross-recognize human or humanized SIRPα protein. However, human SIRPα antibody only detected cells expressing humanized SIRPα protein in B-NDG_hSIRPα homozygous mice (FIGS. 12C-12D).

Consistent with the flow cytometry results using the spleen cells, the mouse SIRPα antibody detected the cells expressing the mouse SIRPα protein in the abdominal cavity washing fluid of B-NDG mice and B-NDG_hSIRPα homozygous mice (FIGS. 13A-13B). However, human SIRPα antibody only detected cells expressing humanized SIRPα protein in the abdominal cavity washing fluid of B-NDG_hSIRPα homozygous mice (FIGS. 13C-13D).

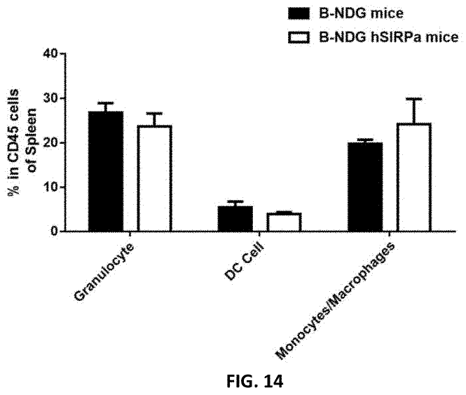

Furthermore, the immune cell subsets in mouse spleen were also detected. As shown in FIG. 14, no obvious difference of leukocyte subsets between B-NDG_hSIRPα mice and B-NDG mice was detected. Specifically, differentiation of granulocytes, dendritic cells (DC), and monocytes/macrophages were not affected.

### Example 6. Preparation of CD47 Gene Humanized Mice

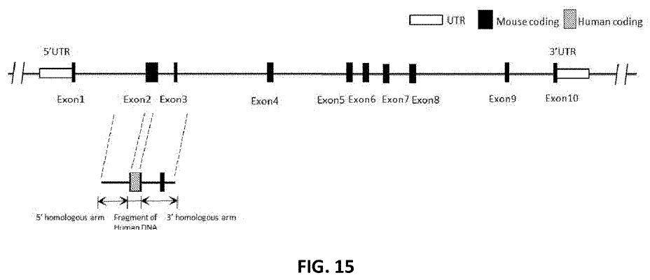

The CRISPR method was used to replace a sequence within exon 2 of mouse CD47 gene (12533-12838 of NCBI reference sequence NC_000082.6, which encodes amino acids 23-124 of NP_034711.1) with a human CD47 gene sequence (SEQ ID NO: 72, which encodes amino acids 23-126 of NP_001768.1). The targeting strategy is shown in FIG. 15, and the resulting CD47 gene humanized mice included the following DNA sequence (a chimeric DNA sequence):

(SEQ ID NO: 73)
tatatgcagattgtaatgaaatatttttgtgtatgtattccaggttcagc
tcaactactgtttaataaaacaaaatctgtagaattcacgttttgtaatg
acactgtcgtcattccatgctttgttactaatatggaggcacaaaacact
actgaagtatacgtaaagtggaaatttaaaggaagagatatCtacacctt
tgatggagctctaaacaagtccactgtccccactgactttagtagtgcaa
aaattgaagtctcacaattactaaaaggagatgcctctttgaagatggat
aagagtgatgctgtctcacacacaggaaactacacttgtgaagtaacaga
attaaccagagaaggtgaaacgatcatagagctgaaaaaccgcacgggta
agtgacacagtttgcctgttttgaaacgtgtgttgagatatggttgccac
tgtgggagtgctgtaaggtggaaccttgcagaagtc

SEQ ID NO: 73 shows only the modified portion of DNA sequence, wherein the italicized underlined region is from human CD47. The capital letter indicates a point mutation.

Mice with humanized CD47 gene (modified exon 2 with human CD47 sequence) were generated. Because the human CD47 gene and the mouse CD47 gene both have multiple variants, the humanized mice can have different humanized CD47 gene variants as well. A non-limiting example of the mRNA of humanized CD47 gene is SEQ ID NO: 74, corresponding to amino acid sequence shown in SEQ ID NO: 75. The same methods described herein can be used to generate other variants of humanized versions of mouse CD47 gene and the transgenic mice containing these variants. The humanized mouse was named as B-NDG hCD47.

### Example 7. Mice with Two or More Humanized Genes

Mice with the humanized SIRPα and/or CD47 gene (e.g., the B-NDG_hSIRPα mouse model using the methods as described in the present disclosure) can also be used to prepare an animal model with double-humanized or multi-humanized genes. For example, the fertilized eggs used in the microinjection and embryo transfer process can be selected from the fertilized eggs of other genetically modified mice, so as to obtain double- or multiple-gene modified mouse models. The fertilized eggs of SIRPα and/or CD47 gene humanized mice can also be further genetically engineered (e.g., by gene editing techniques) to produce mouse lines with one or more humanized or otherwise genetically modified mouse models. In addition, the B-NDG_hSIRPα or B-NDG hCD47 animal model homozygote or heterozygote can be bred with other genetically modified homozygous or heterozygous animal models, and the progeny can be screened. According to the Mendelian law, there is a possibility to obtain the double-gene or multiple-gene modified heterozygous animals, and then the heterozygous animals can be bred with each other to finally obtain the double-gene or multiple-gene modified homozygotes.

In the case of generating double humanized B-NDG_hSIRPα/hCD47 mice, since the mouse CD47 gene and SIRPα gene are located on different chromosomes, the double humanized B-NDG_hSIRPα/hCD47 mouse model was obtained by breeding the B-NDG hCD47 humanized mice with the B-NDG_hSIRPα humanized mice (or through in vitro fertilization), screening, and breeding of positive offspring mice.

A homozygous B-NDG_hSIRPα/hCD47 mouse, a B-NDG mouse, and a wildtype C57BL/6 mouse were selected. Blood samples were collected after euthanasia. According to the method in Example 3, Brilliant Violet 510™ anti-mouse CD45, PE/Cy™ 7 Mouse anti-mouse NK1.1, APC anti-mouse CD11c, V450 Rat anti-mouse CD11b, Brilliant Violet 711™ anti-mouse TCRβ Chain, PE anti-mouse CD19, PerCP anti-mouse Ly-6G/Ly-6C (Gr-1) Antibody, FITC anti-mouse F4/80, and purified anti-mouse CD16/32 were used for staining B cells, T cells, NK Cells, granulocytes, dendritic cells (DC), macrophages, and monocytes for flow cytometry analysis.

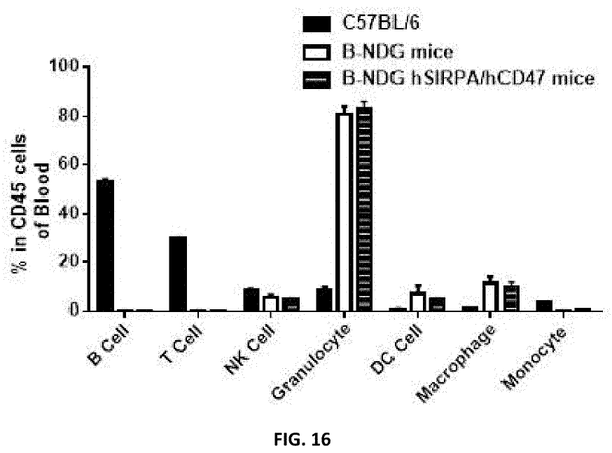

The test results of the blood samples are shown in FIG. 16. The results showed that immune cell differentiation in B-NDG_hSIRPα/hCD47 mice was similar to that of the BNDG mice.

### Example 8. In Vivo Drug Efficacy Verification of B-NDG_hSIRPα/CD47 Mice

B-NDG_hSIRPα/hCD47 mice (8-week old) were intravenously injected (from tail vein) with human B-luciferase-GFP Raji cells (5×10⁵ cells per mouse). When the tumor signal intensity reached about 3.5×10⁶p/sec, the mice were randomly divided to a control group and treatment groups (n=5/group). The treatment groups were treated with an anti-human SIRPα antibody (KWAR23, sequences can be found in Patent Application Publication No. CN106456749A) and/or an anti-human CD20 antibody (Rituximab, sequences can be found in U.S. Pat. No. 7,381,560B2). The doses of the anti-human SIRPα antibody and the anti-human CD20 antibody were 10 mg/kg and 0.1 mg/kg, respectively. The control group (G1) mice were injected with the same volume of PBS. The administration frequency was twice per week (six injections in total). The specific administered antibodies, dose, administration route and frequency are shown in the table below. The mice were measured for their body weight, and tumor fluorescence signal intensity twice a week, and the status of mice were recorded every day after administration. Euthanasia was performed when the mice showed abnormal behavior, paralyzed, or showed a weight reduction of 20% as compared with the weight before administration.

TABLE 19

| | Animal | | Dose | Administration | Administration |
|---|---|---|---|---|---|
| Group | number | Antibody | (mg/kg) | route | frequency |
| G1 | 5 | PBS | — | i.p. | BIW (6 total) |
| G2 | 5 | Rituximab | 0.1 | i.p. | BIW (6 total) |
| G3 | 5 | KWAR23 | 10 | i.p. | BIW (6 total) |
| G4 | 5 | Rituximab | 0.1 | i.p. | BIW (6 total) |
| | | KWAR23 | 10 | i.p. | BIW (6 total) |

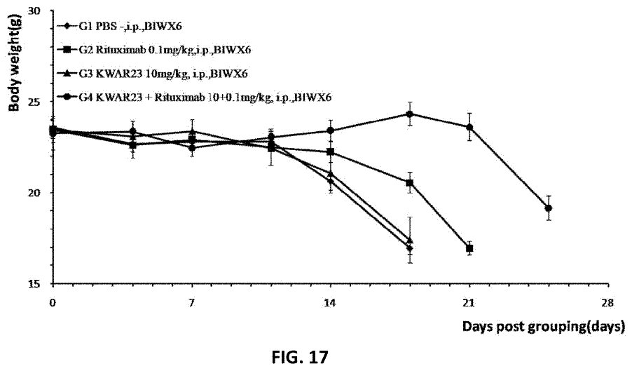

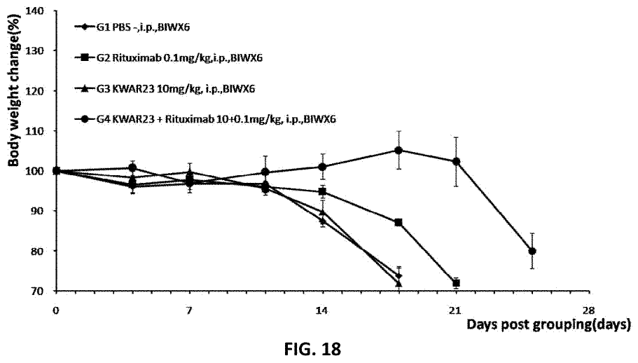

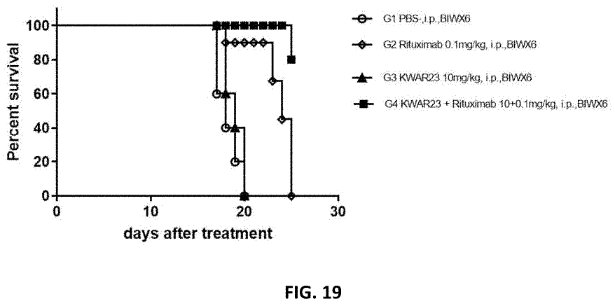

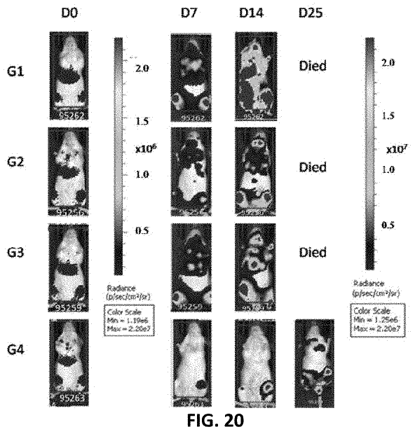

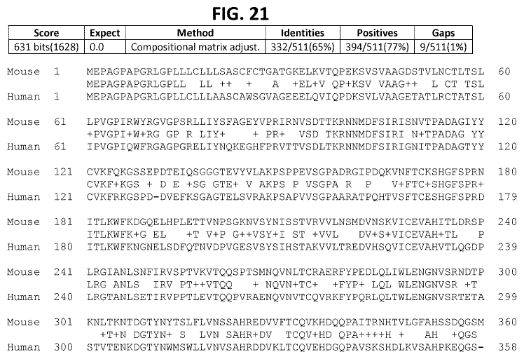

In general, the body weight and body weight change of all the treatment group mice were not significantly different from the control group mice within 11 days after grouping (FIGS. 17-18). The animals in each group were grossly healthy and all survived. 11 days after grouping, except for treatment group G4, mice in groups G1-G3 all showed different degrees of weight loss, and deaths began to occur 18 days after grouping. In particular, the weight loss and death in the G1 group were the most significant (see survival curves in FIG. 19), indicating that injection of Raji cells had a negative impact on mouse health. The body weight of mice in the G4 group increased slowly from 11 days to 18 days after grouping, and significant body weight differences as compared that those of the control group G1 mice at 14 days and 18 days after grouping were observed (p<0.05). At the end of the experiment (25 days after grouping), 4 mice in the G4 group were still alive (80% survival rate), and the body weight of the survived mice did not decrease significantly. This indicates that the animals tolerated the two antibodies or the antibody combination well, and the antibodies did not cause obvious toxic effects on the animals. However, according to tumor fluorescence signal intensity (FIG. 20), the signal intensity of mice in the control group continued to increase during the experimental period, indicating that the tumors continued to grow during the experimental period and metastasized obviously. But the signal intensity of all treatment groups was significantly lower than G1, indicating that the tumor growth in mice in the G2, G3, and G4 groups was inhibited to different degrees after treatment, and the inhibitory effect was G4>G2>G3. This indicates that the anti-CD20 antibody Rituximab is more effective in treating tumors in vivo than the anti-human SIRPα antibody KWAR23, and the combination of the two antibodies can significantly inhibit tumor growth with a better therapeutic effect.

The tumor growth of each group of mice was evaluated. The table below lists the main data and analysis results, specifically including: the tumor fluorescence signal intensity and body weight of mice at the time of grouping (day 0), 7 days after grouping (day 7), 14 days after grouping (day 14), and grouping 25 days (day 25); the survival status of the mice at the end of the experiment (25 days after grouping); and the statistical difference (P value) of the body weight and tumor fluorescence signal intensity between the mice in the treatment group and the control group at day 14. At day 14, tumor fluorescence signal intensity of mice administered with Rituximab alone (G2), KWAR23 (G3) alone, or two antibodies combined was significantly lower than that of the control group (G1). In particular, Rituximab and KWAR23 combined treatment group (G4) showed the lowest fluorescence intensity. FIG. 20 shows the tumor fluorescence image of one survived mouse in each group at the time of grouping (day 0) and 7, 14, or 25 days after grouping. The results indicate that the tumor inhibitory effect of the combination of two monoclonal antibodies was better than that of administration of a single antibody. The results also indicate that combination of the anti-human SIRPα antibody and anti-CD20 antibody can significantly inhibit tumor growth.

TABLE 20
Tumor fluorescence signal intensity, survival rate, and body weight

| | | | | | | | P value (day 14) | |
|---|---|---|---|---|---|---|---|---|
| | | | | | | Survival | Body | Tumor |
| | | Tumor fluorescence signal intensity ± (SD) (p/sec) | | | | stat | weight | fluorescence |
| | | Day 0 | Day 7 | Day 14 | Day 25 | [symbol] | [symbol] | [symbol] |
| Control | | 3.47E+06 | 6.92E+08 | 2.96E+09 | N/A | 0/5 | N/A | N/A |
| group G1 | | (2.69E+05) | (1.25E+08) | (4.4E+08) | | | | |
| Treatment | G2 | 3.47E+06 | 8.71E+07 | 1.30E+09 | 2.35E+09 | 4/5 | 0.063 | 0.007 |
| group | | (2.53E+05) | (2.30E+07) | (1.33E+08) | (7.89E+08) | | | |
| | G3 | 3.49E+06 | 5.92E+08 | 1.82E+09 | NA | 0/5 | 0.711 | 0.05 |
| | | (3.7E+05) | (8.00E+07) | (2.32E+08) | | | | |
| | G4 | 3.47E+06 | 8.50E+07 | 1.31E+08 | 2.24E+08 | 5/5 | 0.006 | 0 |
| | | (4.19E+05) | (2.39E+06) | (3.98E+07) | (6.65E+07) | | | |

Note:
data in parentheses are standard deviation. [symbol]
indicates data missing or illegible when filed

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric SIRPα, wherein the animal is immune-deficient.

2. The animal of claim 1, wherein the genome of the animal comprises a disruption in the animal's endogenous CD132 gene.

3. The animal of claim 1 or 2, wherein the sequence encoding the human or chimeric SIRPα is operably linked to an endogenous regulatory element at the endogenous SIRPα gene locus in the at least one chromosome.

4. The animal of any one of claims 1-3, wherein the sequence encoding a human or chimeric SIRPα comprises a sequence encoding an amino acid sequence that is at least 70%, 75%, 80%, 85%, 90%, 95%, 99%, or 100% identical to human SIRPα (NP_542970.1; SEQ ID NO: 77).

5. The animal of any one of claims 1-3, wherein the sequence encoding a human or chimeric SIRPα comprises a sequence encoding an amino acid sequence that is at least 70%, 75%, 80%, 85%, 90%, 95%, 99%, or 100% identical to SEQ ID NO: 53.

6. The animal of any one of claims 1-3, wherein the sequence encoding a human or chimeric SIRPα comprises a sequence that is at least 70%, 75%, 80%, 85%, 90%, 95%, 99%, or 100% identical to amino acids 31-138 of SEQ ID NO: 77.

7. The animal of any one of claims 1-6, wherein the animal is a mammal, e.g., a monkey, a rodent or a mouse.

8. The animal of any one of claims 1-7, wherein the animal is a mouse.

9. The animal of any one of claims 1-8, wherein the animal does not express endogenous SIRPα.

10. The animal of any one of claims 1-9, wherein the animal has one or more cells expressing human or chimeric SIRPα.

11. The animal of any one of claims 1-10, wherein the genome of the animal comprises a replacement of a sequence encoding a region of endogenous SIRPα with a sequence encoding a corresponding region of human SIRPα at an endogenous SIRPα gene locus.

12. The animal of claim 11, wherein the replaced locus is the extracellular domain of SIRPα.

13. The animal of claim 11, wherein the replaced locus is the extracellular N-terminal IgV domain of SIRPα.

14. The animal of claim 11, wherein the animal is a mouse, and the replaced endogenous SIRPα region is exon 2 of the endogenous mouse SIRPα gene.

15. The animal of claim 11, wherein the sequence encoding a corresponding region of human SIRPα comprises at least 100, 200, or 300 nucleotides of exon 3 of a human SIRPα gene.

16. The animal of any one of claims 11-15, wherein the animal is homozygous with respect to the replacement at the endogenous SIRPα gene locus.

17. The animal of any one of claims 11-15, wherein the animal is heterozygous with respect to the replacement at the endogenous SIRPα gene locus.

18. The animal of any one of claims 2-17, wherein the disruption of the endogenous CD132 gene comprises deletion of exon 2 of the endogenous CD132 gene.

19. The animal of any one of claims 2-18, wherein the disruption of the endogenous CD132 gene comprises deletion of exon 1, or part thereof of the endogenous CD132 gene.

20. The animal of any one of claims 2-19, wherein the disruption of the endogenous CD132 gene further comprises deletion of one or more exons or part of exons selected from the group consisting of exon 3, exon 4, exon 5, exon 6, exon 7, and exon 8 of the endogenous CD132 gene.

21. The animal of any one of claims 2-20, wherein the disruption of the endogenous CD132 gene comprises deletion of exons 1-8 of the endogenous CD132 gene.

22. The animal of any one of claims 2-21, wherein the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene.

23. The animal of any one of claims 2-22, wherein the disruption consists of
deletion of more than 150 nucleotides in exon 1,
deletion of the entirety of intron 1, exon 2, intron 2, exon 3, intron 3, exon 4, intron 4, exon 5, intron 5, exon 6, intron 6, exon 7, intron 7, and
deletion of more than 250 nucleotides in exon 8.

24. The animal of any one of claims 2-23, wherein the animal is homozygous with respect to the disruption of the endogenous CD132 gene.

25. The animal of any one of claims 2-23, wherein the animal is heterozygous with respect to the disruption of the endogenous CD132 gene.

26. The animal of any one of claims 2-25, wherein the disruption prevents the expression of functional CD132 protein.

27. The animal of any one of claims 2-26, wherein the length of the remaining exon sequences at the endogenous CD132 gene locus is less than 30% of the total length of all exon sequences of the endogenous CD132 gene.

28. The animal of any one of claims 2-26, wherein the length of the remaining sequences at that the endogenous CD132 gene locus is less than 15% of the full sequence of the endogenous CD132 gene.

29. The animal of any one of claims 1-28, wherein the animal further comprises a sequence encoding an additional human or chimeric protein (e.g., CD47, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, tumor necrosis factor receptor superfamily member 9 (4-1BB), or TNF Receptor Superfamily Member 4 (OX40)).

30. The animal of claim 29, wherein the additional human or chimeric protein is CD47 and/or PD-1.

31. A genetically-modified, non-human animal whose genome comprises:
at least one chromosome comprising a sequence encoding a human or chimeric CD47, wherein the animal is immune-deficient.

32. The animal of claim 31, wherein the genome of the animal comprises a disruption in the animal's endogenous CD132 gene.

33. The animal of claim 31 or 32, wherein the sequence encoding the human or chimeric CD47 is operably linked to an endogenous regulatory element at the endogenous CD47 gene locus in the at least one chromosome.

34. The animal of any one of claims 31-33, wherein the sequence encoding a human or chimeric CD47 comprises a sequence encoding an amino acid sequence that is at least 70%, 75%, 80%, 85%, 90%, 95%, 99%, or 100% identical to human CD47 (NP_001768.1; SEQ ID NO: 81).

35. The animal of any one of claims 31-33, wherein the sequence encoding a human or chimeric CD47 comprises a sequence encoding an amino acid sequence that is at least 70%, 75%, 80%, 85%, 90%, 95%, 99%, or 100% identical to SEQ ID NO: 75.

36. The animal of any one of claims 31-33, wherein the sequence encoding a human or chimeric CD47 comprises a sequence that is at least 70%, 75%, 80%, 85%, 90%, 95%, 99%, or 100% identical to amino acids 23-126 of SEQ ID NO: 81.

37. The animal of any one of claims 31-36, wherein the animal is a mammal, e.g., a monkey, a rodent or a mouse.

38. The animal of any one of claims 31-37, wherein the animal is a mouse.

39. The animal of any one of claims 31-38, wherein the animal does not express endogenous CD47.

40. The animal of any one of claims 31-39, wherein the animal has one or more cells expressing human or chimeric CD47.

41. The animal of any one of claims 31-40, wherein the genome of the animal comprises a replacement of a sequence encoding a region of endogenous CD47 with a sequence encoding a corresponding region of human CD47 at an endogenous CD47 gene locus.

42. The animal of claim 41, wherein the replaced locus is the extracellular N-terminal IgV domain of CD47.

43. The animal of claim 41, wherein the animal is a mouse, and the replaced endogenous CD47 region is exon 2 of the endogenous mouse CD47 gene.

44. The animal of claim 41, wherein the sequence encoding the corresponding region of CD47 comprises at least 100, 200, or 300 nucleotides of exon 2 of a human CD47 gene.

45. The animal of any one of claims 31-44, wherein the animal is homozygous with respect to the replacement at the endogenous SIRPα gene locus.

46. The animal of any one of claims 31-44, wherein the animal is heterozygous with respect to the replacement at the endogenous SIRPα gene locus.

47. The animal of any one of claim 32-46, wherein the disruption of the endogenous CD132 gene comprises deletion of exon 2 of the endogenous CD132 gene.

48. The animal of any one of claims 32-47, wherein the disruption of the endogenous CD132 gene comprises deletion of exon 1 or part thereof of the endogenous CD132 gene.

49. The animal of any one of claims 32-48, wherein the disruption of the endogenous CD132 gene further comprises deletion of one or more exons or part of exons selected from the group consisting of exon 3, exon 4, exon 5, exon 6, exon 7, and exon 8 of the endogenous CD132 gene.

50. The animal of any one of claims 32-49, wherein the disruption of the endogenous CD132 gene comprises deletion of exons 1-8 of the endogenous CD132 gene.

51. The animal of any one of claims 32-50, wherein the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene.

52. The animal of any one of claims 32-51, wherein the disruption consists of
deletion of more than 150 nucleotides in exon 1,
deletion of the entirety of intron 1, exon 2, intron 2, exon 3, intron 3, exon 4, intron 4, exon 5, intron 5, exon 6, intron 6, exon 7, intron 7, and
deletion of more than 250 nucleotides in exon 8.

53. The animal of any one of claims 32-52, wherein the animal is homozygous with respect to the disruption of the endogenous CD132 gene.

54. The animal of any one of claims 32-52, wherein the animal is heterozygous with respect to the disruption of the endogenous CD132 gene.

55. The animal of any one of claims 32-54, wherein the disruption prevents the expression of functional CD132 protein.

56. The animal of any one of claims 32-55, wherein the length of the remaining exon sequences at the endogenous CD132 gene locus is less than 30% of the total length of all exon sequences of the endogenous CD132 gene.

57. The animal of any one of claims 32-55, wherein the length of the remaining sequences at that the endogenous CD132 gene locus is less than 15% of the full sequence of the endogenous CD132 gene.

58. The animal of any one of claims 31-57, wherein the animal further comprises a sequence encoding an additional human or chimeric protein (e.g., SIRPα, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, tumor necrosis factor receptor superfamily member 9 (4-1BB), or TNF Receptor Superfamily Member 4 (OX40)).

59. The animal of claim 58, wherein the additional human or chimeric protein is SIRPα and/or PD-1.

60. The animal of any one of claims 1-59, wherein the animal does not have functional T cells or B cells.

61. A method of determining effectiveness of an agent targeting the CD47/SIRPα axis for the treatment of cancer, comprising:
administering the agent to the animal of any one of claims 1-60, wherein the animal has a tumor; and
determining the inhibitory effects of the agent to the tumor.

62. The method of claim 61, wherein the agent comprises or consists of an anti-SIRPα antibody and/or an anti-CD47 antibody.

63. The method of claim 61 or 62, wherein the animal comprises one or more tumor cells that express CD47.

64. The method of any one of claims 61-63, wherein the tumor comprises one or more cancer cells that are injected into the animal.

65. The method of any one of claims 61-64, wherein determining the inhibitory effects of the anti-SIRPα antibody to the tumor involves measuring the tumor volume in the animal.

66. The method of any one of claims 61-65, wherein the tumor cells are melanoma cells, non-small cell lung carcinoma (NSCLC) cells, small cell lung cancer (SCLC) cells, non-Hodgkin lymphoma cells, bladder cancer cells, prostate cancer cells, breast cancer cells, ovarian cancer cells, colorectal cancer cells, and/or refractory solid tumor cells.

67. A method of determining effectiveness of an agent and an additional therapeutic agent for the treatment of a tumor, comprising
administering the agent and the additional therapeutic agent to the animal of any one of claims 1-60, wherein the animal has a tumor; and
determining the inhibitory effects on the tumor.

68. The method of claim 67, wherein the agent is an anti-SIRPα antibody and/or the anti-CD47 antibody.

69. The method of claim 67 or 68, wherein the additional therapeutic agent is an anti-PD-1 antibody, an anti-PD-L1 antibody, an anti-CTLA4 antibody, an anti-CD20 antibody, an anti-EGFR antibody, or an anti-CD319 antibody.

70. A method of determining toxicity of an agent, the method comprising administering the anti-SIRPα antibody or the anti-CD47 antibody to the animal of any one of 1-60; and
determining weight change of the animal.

71. The method of claim 70, wherein the agent is an anti-SIRPα antibody or an anti-CD47 antibody.

72. The method of claim 70 or 71, the method further comprising performing a blood test (e.g., determining red blood cell count).

73. A method of evaluating the effect of an agent targeting the CD47/SIRPα axis on phagocytosis, comprising:
administering the agent to the animal of any one of claims 1-60, wherein the animal has a tumor.

74. The method of claim 73, wherein phagocytosis is induced by granulocytes (e.g., neutrophils, basophils, eosinophils, or mast cells) or macrophages.

75. The method of claim 73 or 74, wherein the agent is an anti-SIRPα antibody and/or an anti-CD47 antibody.

76. A method of producing an animal comprising a human hemato-lymphoid system, the method comprising:
engrafting a population of cells comprising human hematopoietic cells or human peripheral blood cells into the animal of any one of claims 1-60.

77. The method of claim 76, wherein the human hemato-lymphoid system comprises human cells selected from the group consisting of hematopoietic stem cells, myeloid precursor cells, myeloid cells, dendritic cells, monocytes, granulocytes, neutrophils, mast cells, lymphocytes, and platelets.

78. The method of claim 76 or 77, further comprising: irradiating the animal prior to the engrafting.

79. A protein comprising an amino acid sequence, wherein the amino acid sequence is one of the following:
(a) an amino acid sequence set forth in SEQ ID NO: 53, 62, or 75;
(b) an amino acid sequence that is at least 90%, 91%, 92%, 93%, 94%, 95%, 96%, 97%, 98%, or 99% identical to SEQ ID NO: 53, 62, or 75;
(c) an amino acid sequence that is different from the amino acid sequence set forth in SEQ ID NO: 53, 62, or 75 by no more than 10, 9, 8, 7, 6, 5, 4, 3, 2 or 1 amino acid; and
(d) an amino acid sequence that comprises a substitution, a deletion and/or insertion of one, two, three, four, five or more amino acids to the amino acid sequence set forth in SEQ ID NO: 53, 62, or 75.

80. A nucleic acid comprising a nucleotide sequence, wherein the nucleotide sequence is one of the following:
(a) a sequence that encodes the protein of claim 79;
(b) SEQ ID NO: 18, 50, 51, or 52;
(c) SEQ ID NO:72, 73 or 74; and
(d) a sequence that is at least 90%, 91%, 92%, 93%, 94%, 95%, 96%, 97%, 98%, or 99% identical to SEQ ID NO: 18, 50, 51, 52, 72, 73, or 74.

81. A cell comprising the protein of claim 79 and/or the nucleic acid of claim 80.

82. An animal comprising the protein of claim 79 and/or the nucleic acid of claim 80.
